(19) United States
(12) Patent Application Publication
(10) Pub. No.: US 2023/0043518 A1
(43) Pub. Date: Feb. 9, 2023

(54) CANCER IMMUNOTHERAPY

(71) Applicant: AXELIA ONCOLOGY PTY LTD, Melbourne, Victoria (AU)

(72) Inventors: Christophe Demaison, Melbourne (AU); Christopher Smith, Melbourne (AU); Francesca Mercuri, Melbourne (AU); Tobias Bald, Melbourne (AU); Michael Bettess, Melbourne (AU)

(21) Appl. No.: 17/640,704

(22) PCT Filed: Sep. 4, 2020
(86) PCT No.: PCT/AU2020/050932

§ 371 Date: Mar. 4, 2022

(30) Foreign Application Priority Data
Sep. 4, 2019 (AU) 2019903262
Dec. 20, 2019 (AU) 2019904864

(51) Int. Cl. A61K 39/395; A61K 47/60; A61K 47/54; A61P 35/00

(52) U.S. Cl. CPC A61K 39/3955; A61K 47/60; A61K 47/542; A61P 35/00

(57) ABSTRACT

Methods, compounds, compositions and kits for the treatment and/or prevention of cancer are provided. In particular, methods for the treatment of cancer comprising the administration of a TLR2 agonist, such as a conjugate of dipalmitoyl-S-glyceryl-cysteine (Pam2Cys) and polyethylene glycol (PEG), more particularly a Pam2Cys-Ser-PEG compound, and an immunostimulant such as an anti-PD-1, anti-PDL-1, anti-PL-1, or anti-CTLA-4 immunotherapeutic, are provided.

### CROSS REFERENCE TO EARLIER APPLICATIONS

This application claims priority to Australian provisional patent application nos. AU2019903262 (filed on 4 Sep. 2019) and Australian provisional patent application no. AU2019904864 (filed on 20 Dec. 2019). The entire contents of each of AU2019903262 and AU2019904864 is hereby incorporated by reference.

### FIELD OF THE INVENTION

The invention relates to methods, compounds, compositions and kits for the treatment and/or prevention of cancer. In one aspect, the invention relates to the use of an immunotherapy for the treatment and/or prevention of cancer.

### BACKGROUND OF THE INVENTION

Immunotherapies have shown promise for the treatment of cancer due to their ability to slow the growth and spread of cancer cells, and by helping the immune system destroy existing cancer cells. Immunotherapies may assist the immune system by priming and boosting the immune system via stimulation of antigen-presenting cells, T-cells, or innate cells, by reducing immunosuppression in the tumor environment by regulating inhibitory pathways and/or by enhancing adaptive or innate immune responses.

An example of an immunotherapy is checkpoint inhibitors. Checkpoint inhibitors currently approved by the US Food and Drug Administration (FDA) target the cytotoxic T-lymphocyte-associated protein 4 (CTLA-4), programmed cell death receptor 1 (PD-1), or programmed cell death ligand 1 (PD-L1). Such checkpoint inhibitors work by preventing immune evasion from cancer cells. The first approved agent, ipilimumab, received FDA marketing authorization in 2011 for metastatic melanoma. Since ipilimumab, five more checkpoint inhibitor drugs have been approved for a total of 14 different indications.

Between 2015 and 2017, the number of clinical trials using PD-1 and PD-L1 inhibitors has increased nearly 600%. Even though the number of indications for which checkpoint inhibitors have been administered has increased substantially in recent years, the increase in benefit from these drugs in terms of the percentage of patients responding has slowed. Whilst clinical responses to PD-1 immunotherapy positively correlate with tumor PD-L1 expression, along with other predictive biomarkers such as preexisting CD8+ T cell infiltration and mutational/neoantigen burden, in other clinical trials, it has been demonstrated that some patients with tumors that do not express PD-L1 respond to PD-1 pathway blockade. In fact, recent analyses suggest that checkpoint inhibitors may at best lead to responses among less than 13% of patients with various types of cancer in the United States.

There therefore remains a need for improved immunotherapies for the treatment of cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method of treating, preventing or minimising progression of cancer in a subject comprising administering a therapeutically effective amount of a TLR2 agonist and an immunostimulant to a subject, thereby treating, preventing or minimising progression of cancer in the subject.

In any aspect of the invention, the TLR2 agonist may be any one as described herein.

Preferably, the TLR2 agonist is a compound as defined by any one of formulas (I), (IA1), (IA2), (II), (III), (IV), (V), (VI), (VII), (VIII), (IX), (X), (X), (XII), (XIII), (XIV), (XV), (XVI), (XVII), (XVIII) and (XIX) (collectively referred to herein as formulas (I)-(XIX)).

In any aspect of the invention, the TLR2 agonist may be a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue.

In any aspect of the invention, the compound may comprise or consist of partial structure A1Y′ or A2Y′:

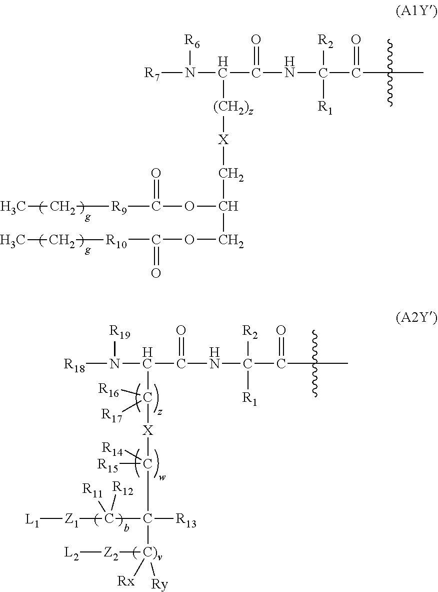

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

b and w are each independently an integer from 0 to 7 and v is an integer from 0 to 5, provided that:

the sum of b, v, and w is at least 3; and

the sum of b and w is from 0 to 7;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A2;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and

A1Y′ or A2Y′ is covalently linked to polyethylene glycol (PEG),

or a pharmaceutically acceptable salt, solvate or prodrug thereof.

In some embodiments, the TLR2 agonist is a compound selected from any of compounds 001-010, A101-A114 and A201-A232.

Reference to a “compound of the invention” as used herein may refer to any of:
a compound of formulas (I)-(XIX);
a compound selected from any of compounds 001-010, A101-A114 and A201-A232;
a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue; and/or
a compound comprising a partial structure of formula (A1Y′) or (A2Y′) covalently linked to a PEG.

In any aspect of the invention, the immunostimulant may be selected from any one described herein, including, but not limited to, the group consisting of:
cellular immunotherapies (cytotoxic cellular immunotherapies or adoptive cellular therapies);
oncolytic viruses;
cancer vaccines;
T-cell engagers;
bispecific T-cell engagers; and
checkpoint inhibitors.

In a preferred embodiment, the immunostimulant to be used in accordance with any method described herein is a checkpoint inhibitor.

In another aspect, the present invention provides a method of treating, preventing or minimising progression of cancer in a subject comprising administering a therapeutically effective amount of a TLR2 agonist and a checkpoint inhibitor to a subject, thereby treating, preventing or minimising progression of cancer in the subject.

In another aspect, the present invention provides a method of treating, preventing or minimising the progression of cancer in a subject who has received, or who is receiving, an immunostimulant comprising administering a therapeutically effective amount of a TLR2 agonist to a subject, thereby treating, preventing or minimising progression of cancer in the subject.

In another aspect, the present invention provides a method of treating, preventing or minimising the progression of cancer in a subject who has received, or who is receiving, a checkpoint inhibitor comprising administering a therapeutically effective amount of a TLR2 agonist to a subject, thereby treating, preventing or minimising progression of cancer in the subject.

In any aspect of the invention, the TLR2 agonist and the immunostimulant may be administered at the same time. Alternatively, they may be administered sequentially. For instance, the immunostimulant may be administered prior to the TLR2 agonist or the TLR2 agonist may be administered prior to the immunostimulant. Alternatively, treatment with the immunostimulant and/or TLR2 agonist may be staggered. In a preferred embodiment, the immunostimulant is a checkpoint inhibitor. In this aspect of the invention, the TLR2 agonist may be administered once or twice weekly and the checkpoint inhibitor may be administered once every three weeks.

In another aspect, the present invention provides a method of treating, preventing or minimising progression of cancer in a subject comprising the steps of:
identifying a subject having cancer who has received, or who is receiving an immunostimulant for the treatment of cancer,
assessing whether the subject is responsive to the immunostimulant,
administering a therapeutically effective amount of a TLR2 agonist to the subject if the subject is not responsive to the immunostimulant, thereby treating, preventing or minimising progression of cancer in the subject.

In a preferred embodiment, the immunostimulant is a checkpoint inhibitor, preferably a PD-1 inhibitor, a PD-L1 inhibitor or a CTLA-4 inhibitor. More preferably, the checkpoint inhibitor is a PD-1, PD-L1 or CTLA-4 antibody.

In another aspect, the present invention provides a method of treating, preventing or minimising progression of cancer in a subject comprising the steps of:
identifying a subject having cancer and being unresponsive to a treatment comprising an immunostimulant,
administering a therapeutically effective amount of a compound comprising, consisting or consisting essentially of a TLR2 agonist to the subject, thereby treating, preventing or minimising progression of cancer in the subject.

In another aspect, the present invention provides a method of treating, preventing or minimising progression of cancer in a subject comprising the steps of:
identifying a subject having cancer; and
administering a therapeutically effective amount of a TLR2 agonist and an immunostimulant to the subject,

thereby treating, preventing or minimising progression of cancer in the subject.

In another aspect, the present invention provides a method of treating, preventing or minimising progression of cancer in a subject comprising the steps of:
identifying a subject having cancer; and
administering a therapeutically effective amount of a TLR2 agonist and a checkpoint inhibitor to the subject,

thereby treating, preventing or minimising progression of cancer in the subject.

In any aspect of the invention, the TLR2 agonist may be administered in a composition.

Further, in any aspect of the invention, the immunostimulant may be administered in a composition. In a preferred embodiment, the checkpoint inhibitor may be administered in a composition. Typically, the composition further comprises a pharmaceutically acceptable carrier, diluent or excipient.

In any embodiment of the invention, the composition may be formulated for intravenous administration to the subject. In other words, the composition is suitable for administration intravenously. In another embodiment of the invention, the composition is formulated for administration to the respiratory tract, preferably by inhalation or intranasal. In another preferred embodiment, the composition is formulated as a nasal spray or as nasal drops. In another embodiment, the TLR2 agonist is formulated for administration to the respiratory tract, preferably by inhalation, and the immunostimulant, preferably any checkpoint inhibitor described herein, is formulated for administration intraperitoneally or intravenously. In this embodiment, the immunostimulant and checkpoint inhibitor may be administered at the same time or at different times.

In one embodiment, the TLR2 agonist is administered in the form of a TLR2 composition, which may be free of compounds that are agonists of other TLRs. Preferably, the only TLR agonist present in the TLR2 composition is an agonist of TLR2 homodimers or heterodimers. Preferably, the composition only contains one TLR2 agonist.

In any embodiment, the composition comprises, consists essentially, or consists of a TLR2 agonist and an immunostimulant, preferably a checkpoint inhibitor, and a pharmaceutically acceptable carrier, diluent or excipient.

In another aspect, the present invention further provides a composition comprising, consisting essentially of or consisting of a TLR2 agonist and an immunostimulant, preferably a checkpoint inhibitor, and a pharmaceutically acceptable carrier, diluent or excipient.

In another aspect, the present invention further provides a method of increasing survival of a subject having cancer comprising administering a therapeutically effective amount of a TLR2 agonist and an immunostimulant to a subject, thereby increasing survival of the subject having cancer.

In another aspect, the present invention further provides a method of increasing survival of a subject having cancer comprising administering a therapeutically effective amount of a TLR2 agonist and a checkpoint inhibitor to a subject, thereby increasing survival of the subject having cancer.

In another aspect, the present invention further provides a method of minimising, reducing or preventing growth of a tumour in a subject having cancer comprising administering a therapeutically effective amount of a TLR2 agonist and an immunostimulant to a subject, thereby minimising, reducing or preventing growth of a tumour in the subject having cancer.

In another aspect, the present invention further provides a method of minimising, reducing or preventing growth of a tumour in a subject having cancer comprising administering a therapeutically effective amount of a TLR2 agonist and a checkpoint inhibitor to a subject, thereby minimising, reducing or preventing growth of a tumour in the subject having cancer.

In another aspect, the present invention further provides a method of minimising, reducing or preventing metastasis in a subject having cancer comprising administering of a therapeutically effective amount of a TLR2 agonist and an immunostimulant to a subject, thereby minimising, reducing or preventing metastasis in the subject having cancer. In a preferred embodiment, the method minimises, reduces or prevents metastasis to the lung.

In another aspect, the present invention further provides a method of minimising, reducing or preventing metastasis in a subject having cancer comprising administering of a therapeutically effective amount of a TLR2 agonist and a checkpoint inhibitor to a subject, thereby minimising, reducing or preventing metastasis in the subject having cancer. In a preferred embodiment, the method minimises, reduces or prevents metastasis to the lung.

In any embodiment, the invention further provides a method of minimising, reducing or preventing cancer in a subject comprising:
identifying a subject having a tumour capable of metastasising; and
administering a therapeutically effective amount of a TLR2 agonist and an immunostimulant to a subject,
   thereby minimising, reducing or preventing cancer in the subject.

In any embodiment, the invention further provides a method of minimising, reducing or preventing cancer in a subject comprising:
identifying a subject having a tumour capable of metastasising; and
administering a therapeutically effective amount of a TLR2 agonist and a checkpoint inhibitor to a subject,
   thereby minimising, reducing or preventing cancer in the subject.

In any embodiment, the invention further provides a method of minimising, reducing or preventing metastasis in a subject having cancer comprising:
identifying a subject having a primary tumour capable of metastasising;
removing the primary tumour from the subject; and
administering a therapeutically effective amount of a TLR2 agonist and an immunostimulant to a subject,
   thereby minimising, reducing or preventing metastasis in the subject having cancer.

In any embodiment, the invention further provides a method of minimising, reducing or preventing metastasis in a subject having cancer comprising:
identifying a subject having a primary tumour capable of metastasising;
removing the primary tumour from the subject; and
administering a therapeutically effective amount of a TLR2 agonist and a checkpoint inhibitor to a subject,
   thereby minimising, reducing or preventing metastasis in the subject having cancer.

In another aspect, the present invention further provides a method of minimising, reducing or preventing growth of a tumour in at least one site distant from the site of the primary tumour in a subject comprising administering a therapeutically effective amount of a TLR2 agonist and an immunostimulant to a subject, thereby minimising, reducing or preventing minimising, reducing or preventing growth of a tumour in at least one site distant from the site of the primary tumour in the subject.

In another aspect, the present invention further provides a method of minimising, reducing or preventing growth of a tumour in at least one site distant from the site of the primary tumour in a subject comprising administering a therapeutically effective amount of a TLR2 agonist and a checkpoint inhibitor to a subject, thereby minimising, reducing or preventing minimising, reducing or preventing growth of a tumour in at least one site distant from the site of the primary tumour in the subject.

In any aspect of the invention, the methods described herein further comprise identifying a subject having cancer. In an embodiment, the cancer may be pre-cancerous or non-metastatic.

In another embodiment, the cancer may be malignant or metastatic.

In another aspect, the present invention further provides use of a compound comprising, consisting or consisting essentially of a TLR2 agonist and an immunostimulant in the preparation of a medicament for treating, preventing or minimising progression of cancer in a subject.

In another aspect, the present invention further provides use of use of a compound comprising, consisting or consisting essentially of a TLR2 agonist in the manufacture of a first medicament, and an immunostimulant in the preparation of a second medicament, wherein the first and second medicaments are for:
treating, preventing or minimising progression of cancer in a subject,
minimising, reducing or preventing growth of a tumour in a subject,
minimising, reducing or preventing metastasis in a subject, or
increasing survival of a subject.

Alternatively, the first and second medicaments are for any other method or use of the invention as described herein.

In another aspect, the present invention further provides use of a TLR2 agonist and an immunostimulant for treating, preventing, or preventing progression of cancer in a subject.

In another aspect, the present invention further provides a TLR2 agonist and an immunostimulant for use in treating, preventing, or preventing progression of cancer in a subject. Alternatively, the TLR2 agonist and the checkpoint inhibitor is for use in any other method or use of the invention as described herein including treating, preventing or minimising progression of cancer in a subject, minimising, reducing or preventing growth of a tumour in a subject, minimising, reducing or preventing metastasis in a subject, or increasing survival of a subject.

In another aspect, the present invention further provides the use of a TLR2 agonist in the manufacture of a medicament for:
treating, preventing or minimising progression of cancer in a subject who has received or who is receiving an immunostimulant,
minimising, reducing or preventing growth of a tumour in a subject who has received or who is receiving an immunostimulant,
minimising, reducing or preventing metastasis in a subject who has received or who is receiving an immunostimulant, or
increasing survival of a subject who has received or who is receiving an immunostimulant.

In any aspect of the invention, any medicament described herein is suitable for administration intraperitoneally, intratumorally, topically, orally, intravenously, to the respiratory tract, preferably by inhalation or intranasally, subcutaneously or intramuscularly. Preferably, any medicament described herein is suitable for administration intravenously or by inhalation. In this aspect, the medicament may be formulated as a nasal spray or as nasal drops.

In another aspect, the present invention further provides a TLR2 agonist for use in treating, preventing or minimising progression of cancer in a subject who has received or who is receiving an immunostimulant. Alternatively, the TLR2 agonist is for use in any other method or use of the invention as described herein. In an aspect of the invention, the TLR2 agonist for use is suitable for administration intraperitoneally, intratumorally, topically, orally, to the respiratory tract, preferably by inhalation or intranasally, intravenously, subcutaneously or intramuscularly. Preferably, the TLR2 agonist for use is suitable for administration intravenously or by inhalation. In another aspect, the TLR2 agonist for use may be formulated as a nasal spray or as nasal drops for intranasal administration.

In another aspect, the present invention further provides use of a TLR2 agonist for treating, preventing or minimising progression of cancer in a subject who has received or who is receiving an immunostimulant. Alternatively, the use of a TLR2 agonist is in any other method or use of the invention as described herein. In an aspect of the invention, the use of a TLR2 agonist is suitable for administration intraperitoneally, intratumorally, topically, orally, to the respiratory tract, preferably by inhalation or intranasally, intravenously, subcutaneously or intramuscularly. Preferably, the use of a TLR2 agonist is suitable for administration intravenously or by inhalation.

In another aspect, the use of a TLR2 agonist may be formulated as a nasal spray or as nasal drops.

In any embodiment of the invention, the immunostimulant is selected from the group consisting of:
cellular immunotherapies (cytotoxic cellular immunotherapies or adoptive cellular therapies);
oncolytic viruses;
cancer vaccines;
T-cell engagers;
bispecific T-cell engagers; and
checkpoint inhibitors.

In any aspect of the invention, the method does not include administration of an:

an antigen;

a peptide antigen; or

a T-helper antigen.

In any aspect of the invention, the TLR2 agonist is not administered as part of a vaccine formulation, typically when administered via the subcutaneous, inhalation, intranasal, intradermal or intramuscular routes.

In any aspect of the invention, the TLR2 agonist is not administered with an antigen. In another aspect, the TLR2 agonist is not administered with a cell penetrating peptide.

In any aspect of the invention, the TLR2 agonist is not Pam3Cys.

In any embodiment of the invention, the effect of any TLR2 agonist and immunostimulant described herein may be significant compared to the effect of the TLR2 agonist alone or the immunostimulant alone. In an aspect, the effect may be additive or synergistic.

In another aspect, when any TLR2 agonist and immunostimulant described herein are administered to a subject, the TLR2 agonist can improve the effectiveness of any immunostimulant described herein. Preferably, the improved effectiveness of any immunostimulant described herein is in relation to a tumour that is partially or completely resistant to a checkpoint inhibitor, for example a PD-1 resistant tumour.

In an embodiment, the effect of any TLR2 agonist and immunostimulant described herein on survival of the subject may be significantly greater than the effect of the TLR2 agonist and immunostimulant when administered alone. In a further embodiment, the effect of any TLR2 agonist and immunostimulant described herein on tumour growth or metastasis in the subject may be significantly greater than the effect of the TLR2 agonist and immunostimulant when administered alone.

In any aspect of the invention, the TLR2 agonist and/or immunostimulant are administered once. In another embodiment, the TLR2 agonist and/or immunostimulant are administered two, three, four or more times to the subject.

In any aspect of the invention, the TLR2 agonist and/or immunostimulant may be administered in the same composition or in separate compositions. In another aspect, the TLR2 agonist and/or immunostimulant may therefore be administered together or sequentially.

Alternatively, administered may be staggered. The TLR2 agonist and/or immunostimulant may also be administered at the same frequency or at different frequencies.

In any aspect of the invention, the TLR2 agonist and the immunostimulant may be administered by any known administration routes in the art including intraperitoneally, intratumorally, topically, orally, to the respiratory tract via inhalation or intranasally, intravenously, subcutaneously or intramuscularly. Preferably, TLR2 agonist and/or immunostimulant are administered intravenously or by inhalation.

In any aspect of the invention, the amount of TLR2 agonist administered may be in the range of about 250 nmoles/kg body weight/dose to 0.005 nmoles/kg body weight/dose.

Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound.

In any aspect of the invention, the amount of TLR2 agonist administered may be in the range of about 0.1, 0.2, 0.3, 0.4, 0.5, 0.6, 0.7, 0.8, 0.9, 1.0, 5, 10, 15, 20, 25, 30, 35, 40, 45, 50, 55, 60, 65, 70, 75, 80, 85, 90, 95 or 100 μg/kg or more.

In any aspect of the invention, the amount of immunostimulant, particularly the amount of checkpoint inhibitor administered may be in the range of from about 0.01 to about 20 mg/kg, about 0.1 to about 10 mg/kg, about 0.1 to about 5 mg/kg, about 1 to about 5 mg/kg, about 2 to about 5 mg/g, about 7.5 to about 12.5 mg/kg, or about 0.1 to about 30 mg/kg of the subject's body weight. For example, dosages can be about 0.1, about 0.3, about 1, about 2, about 3, about 5 or about 10 mg/kg body weight, or, about 0.3, about 1, about 2, about 3, or about 5 mg/kg body weight.

In any aspect of the invention, the cancer is selected from the group consisting of breast cancer, colorectal cancer, adenocarcinomas, mesothelioma, bladder cancer, prostate cancer, germ cell cancer, hepatoma/cholongio carcinoma, neuroendocrine cancer, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, fibrosarcoma, vaginal tumours or Wilm's tumour. In a preferred embodiment, the cancer is melanoma, breast cancer or colon cancer.

In any aspect of the invention, the checkpoint inhibitor may be a PD-1, PD-L1 or a CTLA-4 checkpoint inhibitor. In an aspect, the checkpoint inhibitor is an antibody. Preferably, the checkpoint inhibitor is an inhibitor of PD-1, PD-L1 or CTLA-4 in the form of an antibody.

Preferably, the TLR2 agonist is any one described herein, even more preferably Pam₂Cys-Ser-PEG. As used herein, Pam₂Cys-Ser-PEG may be a compound of the following formula:

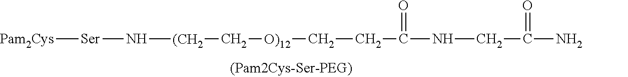

In another preferable aspect, the TLR2 agonist is a compound of the following formula:

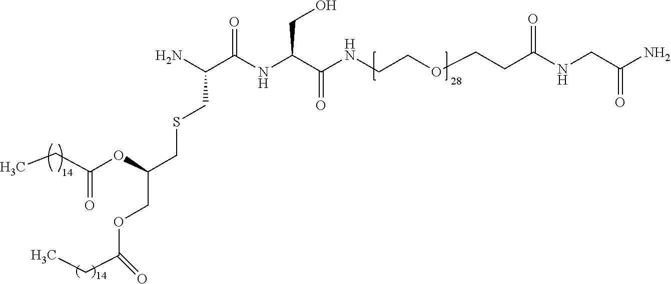

In any embodiment of the invention, the method does not comprise administering agonists of TLRs other than TLR2 homodimers or heterodimers.

In an aspect of the invention, the administration of a therapeutically effective amount of a TLR2 agonist and a therapeutically effective amount of an immunostimulant to a subject comprises administering Pam₂Cys-Ser-PEG and an immunostimulant according to those methods described herein. Preferably, the immunostimulant is a checkpoint inhibitor that is an inhibitor of PD-1, PD-L1 or CTLA-4. More preferably, the checkpoint inhibitor is in the form of an antibody.

In any aspect of the invention, the administration of a therapeutically effective amount of a TLR2 agonist and a therapeutically effective amount of a checkpoint inhibitor to a subject comprises administering compound A108 and an immunostimulant according to those methods described herein. Preferably, the immunostimulant is a checkpoint inhibitor that is an inhibitor of PD-1, PD-L1 or CTLA-4. More preferably, the checkpoint inhibitor is in the form of an antibody. In this or any other aspect, the TLR2 agonist and/or checkpoint inhibitor may be administered to the respiratory tract, preferably by inhalation.

In any aspect of the invention, the TLR2 agonist comprises a lipid, a peptidoglycan, a lipoprotein or a lipopolysaccharide. Preferably, the TLR2 agonist comprises palmitoyl, myristoyl, stearoyl, lauroyl, octanoyl, or decanoyl. The TLR2 agonist may be selected from the group consisting of: Pam2Cys, Pam3Cys, Ste2Cys, Lau2Cys, and Oct2Cys. In a preferred embodiment, the TLR2 agonist comprises Pam2Cys.

In any aspect of the invention, the TLR2 agonist may be conjugated with other compounds or functional groups. Other compounds or functional groups are any of those described herein.

Preferred compounds are selected on the basis to assist in dissolving the TLR2 agonist in a carrier, diluent, excipient or solvent.

Depending on the polarity of the solvent, the solubility of the TLR2 agonist may be increased by a solubilising agent. Therefore, the compound may comprise a TLR2 agonist and a solubilising agent. Preferably, the TLR2 agonist and solubilising agent are linked. The TLR2 agonist may be PEGylated. Preferably, the solubilising agent is any molecule as described herein.

The solubilising agent may comprise, consist essentially, or consist of a positively or negatively charged group. Preferably, the charged group is a branched or linear peptide. Preferably, the positively charged group comprises at least one positively charged amino acid, such as an arginine or lysine residue. Preferably, the negatively charged group comprises at least one negatively charged amino acid, such as glutamate or aspartate. The charged amino acids may be terminal, preferably N-terminal.

Typically, the solubilising agent comprises polyethyleneglycol (PEG) or R4. In any aspect of the invention, the solubilising agent comprises polyethyleneglycol (PEG) and R4.

In any aspect of the invention, the compound comprises Pam2Cys conjugated to PEG₁₁. In any aspect of the invention, the Pam2Cys and PEG₁₁ molecules are separated by at least one serine.

In any aspect of the invention, the TLR2 agonist has improved solubility when compared to the effect of other TLR2 agonists. In a preferred embodiment, the solubility of compound A108 is about 10 fold better than PEG-Pam2Cys-R4.

In an embodiment, compound 1 has a stimulatory effect on human TLR2 at an EC₅₀ between about 0.2 pg/ml and 500 pg/ml or higher. In a preferred embodiment, compound 1 has a stimulatory effect on human TLR2 at an EC₅₀ of 0.5 pg/ml, 1.9 pg/ml, 7.8 pg/ml, 31.25 pg/ml, 125 pg/ml and 500 pg/ml.

In another aspect, compound 1 stimulates TLR2 only at an EC₅₀ of 10 ng/ml. In other words, compound 1 does not stimulate any other TLR2 agonists at an EC₅₀ of 10 ng/ml.

In an embodiment, Pam2CysSK4 has a stimulatory effect on human TLR2 at an EC₅₀ between about 0.05 pg/ml and 64 pg/ml or higher. In another embodiment, Pam2CysSK4 has a stimulatory effect on human TLR2 at an EC₅₀ of 0.5 pg/ml, 1.9 pg/ml, 7.8 pg/ml, 31.3 pg/ml, 125 pg/ml and 500 pg/ml. In another embodiment, Pam2CysSK4 has a stimulatory effect on human TLR2 at an EC₅₀ of 0.0625 pg/ml, 0.25 pg/ml, 1.0 pg/ml, 4 pg/ml, 16 pg/ml and 64 pg/ml.

In an embodiment, compound A108 has a stimulatory effect on human TLR2 at an EC₅₀ between about 0.2 pg/ml and 500 pg/ml or higher.

In any aspect of the invention, any TLR2 agonist described herein does not exhibit cellular cytotoxicity. In an aspect, any TLR2 agonist described herein does not inhibit human cytochrome p450 enzymes.

In a further aspect, any TLR2 agonist described herein does not significantly increase Type I Interferon (IFN-α) and Type II Interferon (IFN-γ). In another aspect, any TLR2 agonist described herein is capable of eliciting an immune response, preferably by increasing monocyte chemoattractant protein-1 (MCP-1).

In another aspect, any TLR2 agonist described herein has a half-life of between about 1 and 10 hours, preferably between about 3 and 6 hours. In a preferred embodiment, the TLR2 agonist is compound A108.

In an aspect, any TLR2 agonist described herein may activate the TLR2 pathway by activating TLR2 homodimers or TLR2/6 heterodimers. In an aspect, compound 1 has a stimulatory effect of human TLR2 homodimers and TLR2/6 heterodimers at concentrations above about 1 pg/ml, preferably above about 2 pg/ml. In an aspect, compound A108 has a stimulatory effect of human TLR2 homodimers at concentrations above about 6 pg/ml, preferably above about 7 pg/ml. In an aspect, compound A108 has a stimulatory effect of human TLR2/6 heterodimers at concentrations above about 0.3 pg/ml, preferably above about 0.4 pg/ml. In another aspect, any TLR2 agonist described herein does not activate the TLR2/1 heterodimer.

A TLR2 agonist contemplated for use in any aspect of the invention is any of the compounds as described herein, including compounds of formulas (I)-(XIX).

In another aspect, the invention provides a method of treating, preventing or minimising progression of cancer in a subject comprising administering a therapeutically effective amount of a compound described herein, such as a compound according to any one of formulas (I)-(XIX) and any other compound comprising a moiety A) and an immunostimulant to a subject, thereby treating, preventing or minimising progression of cancer in the subject.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Compound 1/compound A101 improves the efficacy of anti-PD1 immunotherapy in the MC38 model. A. Experimental setting: MC38-bearing WT mice were treated intratumorally with indicated doses of compound 1 together with anti-PD1 blocking antibodies. Tumour growth was measured and monitored over time. B. Kaplan-Meier survival curves mean±SEM.; n=10 control, n=9 for anti-PD1, anti-PD1+5 pg compound 1 and anti-PD1+50 μg compound 1, n=8 for anti-PD1+25 μg compound 1, statistics; Log rank test).

FIG. 2. Compound 1/compound A101 improves the efficacy of anti-PD1 immunotherapy in the B16F10 model. A. Experimental setting: B16F10-bearing WT mice were treated intratumorally with indicated doses of compound 1 or control together with anti-PD1 blocking antibodies. Tumour growth was measured and monitored over time. B. Kaplan-Meier survival curves (n=10 for control and anti-PD1, n=11 for ant-PD1+25 μg compound 1, n=12 for anti-PD1+50 μg compound 1; Log rank test).

FIG. 3. Systemic delivery of compound 1/compound A101 improves the efficacy of anti-PD1 immunotherapy. A. Experimental setting: MC38-bearing WT mice were treated intravenously with indicated doses of compound 1 or control and with intraperitoneal injections of anti-PD1 or control IgG. B. Tumour growth was measured and monitored over time and assessed in response to compound 1, anti-PD-1, compound 1 and anti-PD-1 or control IgG.

FIG. 4. Combination immunotherapy reduces metastatic spread of 4T1.2 breast cancer cells. A. Experimental setting: Balb/c mice were injected with 4T1.2 breast cancer cells. Primary tumours were surgically removed and mice treated as indicated. The number of metastases were counted on lung surfaces. B. The number of 4T1.2 lung metastasis in indicated cohorts (mean±SEM.; n=8 control and anti-PD1; n=9 for anti-PD1+10 μg compound 1; statistics, One-way ANOVA with Dunnettes test for multiple comparisons).

FIG. 5. Compound A108 in combination with checkpoint inhibitors significantly reduces the growth rate of EMT6.5 tumours in vivo. A. Longitudinal monitoring of EMT6.5 tumours (caliper measurements) demonstrates that compound A108 containing therapeutic regimens leads to a reduction in EMT6.5 tumour growth, and tumour size. (Vehicle vs. anti-PD1 monotherapy (200 μg), p=n.s; Vehicle vs. INNA-042 (10 μg), p=0.0001; Vehicle vs. INNA-042+anti-PD1, p<0.0001; anti-PD1 monotherapy (200 μg) vs. INNA-042+anti-PD1, p<0.0001. B. Kaplan-Meier survival plot indicates better prognosis for animals treated with the combination of compound A108 and anti-PD1. Log-rank test. C. Violin plots detail individual tumour sizes within each group (black dots).

FIG. 6. Compound A108 in combination with checkpoint inhibitors has anti-tumour activity in the immune cell-rich MC38 model. MC38 melanoma bearing WT mice were treated intratumorally with 25 ug of compound A108. Kaplan-Meier survival analysis highlighting the significant improvement in survival in response to the administration of compound A108 and anti-PD1 immunotherapy.

FIG. 7. Compound A108 in combination with checkpoint inhibitors has anti-tumour activity against large tumours of the MC38 colon carcinoma model. Intra-tumoral administration with compound A108 every two days, and with anti-PD1 (10 mg/Kg (200 μg dose) i.p) every four days. Tumour growth was measured and monitored over time. The data shows the anti-tumour effect of compound A108, anti-PD-1 or combination over the treatment period. 2-way ANOVA with Tukey's multiple comparison test.

FIG. 8. Compound A108 in combination with anti-PD-1, anti-PDL1 or anti-CTL4 prevents tumour growth in the WEH1164 fibroblastoma tumour model. A single 2.5 μg dose of compound A108, via the i.p. route on day 7 post-inoculation in combination with A. 3 doses of anti-PD1 (200 μg i.p) or B. anti-CTLA4 (100 μg i.p-day 7) or C. anti-PDL1. A. AntiPD1 vs Anti-PD1+A108 p=0.034; AntiPD1+A108 vs PBS p=0.0081; B. AntiCTLA4+A108 vs AntiCTLA4 p=0.0237; AntiCTLA4+A108 vs A108 alone p=0.0001; AntiCTLA4 alone vs PBS p=0.0453; AntiCTLA4+A108 vs PBS p=0.0001, C. AntiPDL1+A108 vs A108 alone p=0.0001; AntiPDL1 alone vs PBS p=0.0414; AntiPDL1+A108 vs PBS p=0.0028.

FIG. 9. Human TLR2 activity. Human TLR2 activity of compounds A107, A108, A115, A116, A203, A204, A215 and A216 from the NK-κB luciferase assay described in Example 5.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

During the development of a tumour, tumour cells are more or less tolerated by the patient's own immune system, as they are the patient's own cells (e.g., they are self) and are not effectively recognised by the patient's immune system, allowing the tumour cells to grow and divide without proper regulatory control. Accordingly, the patient's own immune system requires stimulation to attack the cancer cells. Cancer immunotherapy involves the utilisation of the immune system of a cancer patient to reject the cancer by stimulating the patient's immune system. In turn, the activated immune system attacks the cancer cells, sparing the normal cells of the patient. A so called immunotherapy that has been shown to be useful for the treatment of cancer is the use of checkpoint inhibitors.

Although such checkpoint inhibitor cancer immunotherapies have demonstrated efficacy in some cancers, such therapies are ineffective in a significant percentage of patients, and some initial responders eventually develop resistance to these therapies with relapsed disease. The ability of a patient to respond to an immunotherapy is dependent upon a large number of factors including individual genetic makeup, history of infection, age, nutritional status, HLA type and consumption of certain medication. Masking of tumour antigens so that the tumour cells cannot be detected by immune surveillance is also a particular problem as is the generally compromised immune status of cancer patients.

By way of example, PD-1 blockade alone has been shown to be ineffective in subsets of patients in some types of cancer such as melanoma and large B cell lymphoma. There is therefore a need for more reliable and efficacious immunotherapy regimes that have utility in the treatment of cancer. The present inventors have unexpectedly found that when an immunostimulant (eg a checkpoint inhibitor) is administered together with a TLR2 agonist, a positive response to the immunotherapy is observed. This effect has been tested in numerous models of cancer including colon cancer, breast cancer, metastatic breast cancer, fibrosarcoma and melanoma. Significantly, in some aspects, the administration of an immunostimulant (eg a checkpoint inhibitor) and a TLR2 agonist is capable of significantly ameliorating the cancer in the subject.

Specifically, when the combination of an immunostimulant and a TLR2 agonist is administered to a subject, this results in significant suppression of tumour growth. This effect is observable even at low doses of a given TLR2 agonist. Significantly, when the combination of an immunostimulant and a TLR2 agonist is administered to a subject this leads to an increase in survival when compared to the effect of the immunostimulant alone. In some cases, the inventors found that whilst the administration of an immunostimulant alone did not increase survival of a subject, there was a significant increase in survival when the immunostimulant was administered in the presence of a TLR2 agonist. Specifically, a positive response is observed when a checkpoint inhibitor is administered together with a TLR2 agonist in models of melanoma and breast cancer, even though the administration of the checkpoint inhibitor alone is less effective or ineffective.

The inventors have shown this remarkable effect in numerous models of cancer of differing aetiology and pathogeneses.

Specifically, the combination of a TLR agonist and an immunostimulant as described herein have been tested in a number of cancer models including:
epithelial mammary carcinoma that has characteristics of triple-negative breast cancer;
metastatic PD1 insensitive breast cancer;
colon carcinoma;
PD1 insensitive melanoma; and
fibrosarcoma.

A skilled person would therefore understand the applicability of the invention to any of the other cancers described herein. The findings described herein are significant as they establish that tumours that were previously untreatable using checkpoint inhibitors alone are now treatable when the combination of an immunostimulant and TLR2 agonist is used.

The inventors also describe herein the utility of a number of different TLR2 agonists in treating cancer in combination with various immunostimulants including:
compound 1/compound A101 in models of colon cancer, melanoma and metastatic breast cancer which was able to impair tumour growth, prolong survival and reduce metastasis when administered intraperitoneally, intratumourally or systemically; and
compound A108 in models of colon cancer, breast cancer, melanoma, fibrosarcoma and metastatic breast cancer, which significantly reduced tumour growth, improved survival rates and reduced metastasis when administered intraperitoneally, intratumourally or systemically.

A skilled person would therefore understand the applicability of the invention to any of the other TLR2 agonists described herein.

The inventors also demonstrate the utility of a number of checkpoint inhibitors in the treatment of cancer in combination with the TLR2 agonists described above including:
PD1;
PD-L1; and
CTLA-4.

A skilled person would therefore understand the applicability of the invention to any of the other immunostimulants described herein or known in the art.

This work therefore identifies a new immunotherapy treatment that may improve response rates in patients with cancer, including those who were previously unresponsive to immunostimulation.

This effect is surprising to the inventors because the effect of TLR2 agonists in treating cancer has been unclear. In particular, it has been reported that subcutaneous or intraperitoneal administration of synthetic TLR2/6 agonists (including Pam2cysSK4 and MALP2) have no anti-tumour activity and to the contrary induce IL-10 and Tregs (Yamazaki et al. PLOS ONE 2011 6(4): e18833). The same group reported that intravenous administration of Pam2cysSK4, a TLR2/6 agonist promotes myeloid derived suppressor cells (Maruyama et al. Biochemical and Biophysical Research Communications 2015 457:445e450). Importantly, it has been suggested that endogenous TLR2/6 agonists derived from cancer cells may enhance metastasis (Kim et al. Nature 2009 457: 102-106). Another study also suggested TLR2 stimulation may promote colorectal cancer cell growth via PI3K/Akt and NFκB signalling pathways (Liu et al. International Immunopharmacology 2018 59:375-383).

Toll-Like Receptors (TLRs) Toll-Like Receptors (TLRs) are pattern recognition receptors (PRRs) expressed by diverse cell types that play an important role in both innate and adaptive immunity. Cells of the innate immune system respond to TLR activation by producing pro-inflammatory cytokines and chemokines that signal for the clearance of the pathogens and damaged-self. Upon engagement with specific ligands, TLR activation leads to the activation of transcription factors such as nuclear factor kappa B (NF)-kB, activating protein-1 (AP-1) and interferon regulatory factors (IRFs) through several adaptor molecules including myeloid differentiation primary response gene 88 (MyD88), Toll-interleukin 1 receptor (TIR) domain containing adaptor protein TIRAP and TIR-domain containing adaptor inducing interferon-beta TRIF, to regulate cytokine expression.

There are a number of TLRs that belong to this membrane receptor protein family including TLR1, TLR3, TLR4, TLR5, TLR6, TLR7, TLR8 and TLR9.

As used herein, the term “TLR2” is intended to mean Toll-Like Receptor 2 protein. In humans, TLR2 is encoded by the TLR2 gene. TLR2 is expressed on the surface of certain cells and plays a fundamental role in pathogen recognition and activation of innate immunity.

A TLR2 agonist is an agent that binds Toll-like receptor 2. The TLR2 agonist may bind to, and activate, TLR2 as a homodimer or heterodimer. Any TLR2 agonist known in the art is contemplated for use in the invention.

In any embodiment of the invention, the TLR2 agonist comprises a lipid, a peptidoglycan, a lipoprotein or a lipopolysaccharide. Preferably, the TLR2 agonist comprises palmitoyl, myristoyl, stearoyl, lauroyl, octanoyl, or decanoyl. The TLR2 agonist may be selected from the group consisting of: Pam2Cys, Pam3Cys, Ste2Cys, Lau2Cys, and Oct2Cys. In a preferred embodiment, the TLR2 agonist comprises Pam2Cys.

An exemplary lipopeptide in accordance with any embodiment of the present invention is the lipopeptide “Pam2Cys”. One of skill in the art would understand that the term “lipopeptide” means any composition of matter comprising one or more lipid moieties and one or more amino acid sequences that are conjugated. “Pam2Cys” (also known as dipalmitoyl-S-glyceryl-cysteine or S-[2,3 bis(palmitoyloxy) propyl] cysteine has been synthesised and corresponds to the lipid moiety of MALP-2, a macrophage-activating lipopeptide isolated from Mycoplasma fermentans. Pam2Cys is known to be a ligand of TLR2.

Pam2Cys has the structure:

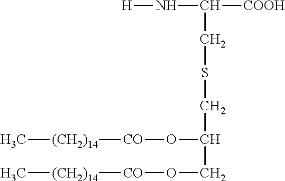

As used herein, reference to “S” as denoted in the above chemical structure defines a sulfur atom.

Another exemplary lipopeptide is the lipoamino acid N-palmitoyl-S-[2,3-bis (palmitoyloxy) propyl] cysteine, also known as Pam3Cys or Pam3Cys-OH is a synthetic version of the N-terminal moiety of Braun's lipoprotein that spans the inner and outer membranes of Gram negative bacteria Pam3Cys has the following structure:

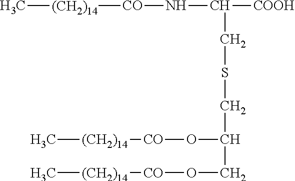

U.S. Pat. No. 5,700,910 describes several N-acyl-S-(2-hydroxyalkyl) cysteines for use as intermediates in the preparation of lipopeptides that are used as synthetic adjuvants, B lymphocyte stimulants, macrophage stimulants, or synthetic vaccines. U.S. Pat. No. 5,700,910 also teaches the use of such compounds as intermediates in the synthesis of Pam3Cys-OH and of lipopeptides that comprise this lipoamino acid or an analog thereof at the N-terminus.

Other lipid moieties which may be used to target cell surface TLRs include palmitoyl, myristoyl, stearoyl, lauroyl, octanoyl, or decanoyl.

In addition to Pam2Cys and Pam3Cys, the present invention also contemplates the use of Ste2Cys, Lau2Cys and Oct2Cys according to the present invention. Those skilled in the art will be aware that Ste2Cys is also known as S-[2,3-bis (stearoyloxy) propyl] cysteine or distearoyl-S-glyceryl-cysteine; that Lau2Cys is also known as S-[2,3-bis (lauroyloxy) propyl] cysteine or dilauroyl-S-glyceryl-cysteine); and that Oct2Cys is also known as S-[2,3- bis (octanoyloxy) propyl] cysteine or dioctanoyl-S-glyceryl-cysteine).

Other suitable TLR2 agonists include, but are not limited to, synthetic triacylated and diacylated lipopeptides, FSL-1 (a synthetic lipoprotein derived from Mycoplasma salivarium 1), Pam3Cys (tripalmitoyl-S-glyceryl cysteine) and S-[2,3- bis(palmitoyloxy)-(2RS)-propyl]-N-palmitoyl-(R)-cysteine, where “Pam3” is “tripalmitoyl-S-glyceryl”. Derivatives of Pam3Cys are also suitable TLR2 agonists, where derivatives include, but are not limited to: S-[2,3-bis(palmitoyloxy)-(2-R,S)-propyl]-N-palmitoyl-(R)-Cys-(S)-Ser-(Lys)4-hydroxytrihydrochloride; Pam3Cys-Ser-Ser-Asn-Ala; Pam3Cys-Ser-(Lys)4; Pam3Cys-Ala-Gly; Pam3Cys-Ser-Gly; Pam3Cys-Ser; Pam3Cys-OMe; Pam3Cys-OH; PamCAG, palmitoyl-Cys((RS)-2,3-di(palmitoyloxy)-propyl)-Ala-Gly-OH, and the like.

Other non-limiting examples of suitable TLR2 agonists are Pam2CSK4 Pam2CysSK4 (dipalmitoyl-S-glyceryl cysteine-serine-(lysine)4; or Pam2Cys-Ser-(Lys)4) is a synthetic diacylated lipopeptide. Other synthetic TLRs agonists include those described, e.g., in Kellner et al. (1992) Biol. Chem. 373:1:51-5; Seifer et al. (1990) Biochem. J, 26:795-802; and Lee et al. (2003) J. Lipid Res., 44:479-486.

A TLR2 agonist may be conjugated with one or more compounds or functional groups.

Examples of particular compounds or functional groups are given below. One form of compound or functional group may act to increase the solubility of the TLR2 agonist. As will be understood by persons skilled in the art, TLR2 agonists are typically non-polar and, accordingly, while being soluble in non-polar solvents, are only less soluble in polar and aqueous solvents. Where it is desired to use the TLR2 agonist in a polar or aqueous solvent, the TLR2 agonist may be conjugated with a solubilising agent.

A solubilising agent may include one, or more than one, solubilising agent which may be conjugated to TLR2 agonist in order to improve the solubility of the TLR2 moiety. The solubilising agent will generally be a polar moiety which increases the solubility of the TLR2 moiety in polar or aqueous solvents.

In any aspect of the invention, the solubilising agent may be a positively charged group. Positively charged groups of the present invention include but are not limited to penetratin, HIV Tat 48-60, HIV Rev 34-50, transportan, oligoarginine peptides (linear and branched), oligolysine peptides, pyrrrochoricin, alpha-helical amphipathic model peptide, polylysine, protamine, FL17, Magnafloc 1697, and the polycationic compounds described in U.S. Pat. Nos. 6,689,478 and 4,035,558.

In yet a further embodiment of the present invention, the solubilising agent comprises, consists essentially of, or consists of a linear or branched peptide. Typically, the linear or branched peptide contains positively or negatively charged amino acids. Positively charged amino acids may be lysine, arginine, histidine, ornithine or combinations thereof. The branched or linear peptide may contain at least one lysine or arginine residue. Preferably, the charged amino acids are terminal, for example N-terminal. The branched peptides may have one of the following structures.

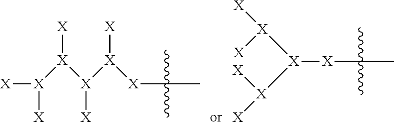

In the above structures X may independently be a charged residue, either a positively or negatively charged residue. Preferably the positively charged amino acids are lysine, arginine, histidine or ornithine. Preferably, the negatively charged amino acids are glutamate or aspartate.

As used herein, ‘PEG’ refers to the polymer compound polyethylene glycol. Unless otherwise defined, reference to ‘PEG’ includes any length polymer of ethylene oxide. Reference to PEG also includes substituted PEG.

The compound or functional group which can act as a solubilising agent may be one or more of the group consisting of “PEG” (or polyethyleneglycol) and a polar polypeptide such as “R4”, a hyper-branched tetra arginine complex; “H4”, a hyper-branched tetra histidine complex; “H8”, a linear peptide containing histidine residues; and “E8” a linear peptide containing glutamate residues. Other linear and branched lipid solubilising agents are also envisaged, including a hyper-branched peptide containing glutamate residues (see, e.g., “branched E8”, below). In yet a further embodiment of the present invention, the solubilising agent includes PEG and one or more of the group consisting of R4, H4, H8 and E8 (linear or branched). R4, H4, H8 and E8 have been previously described in PCT/AU2009/000469 (WO/2010/115230) and have the following structures:

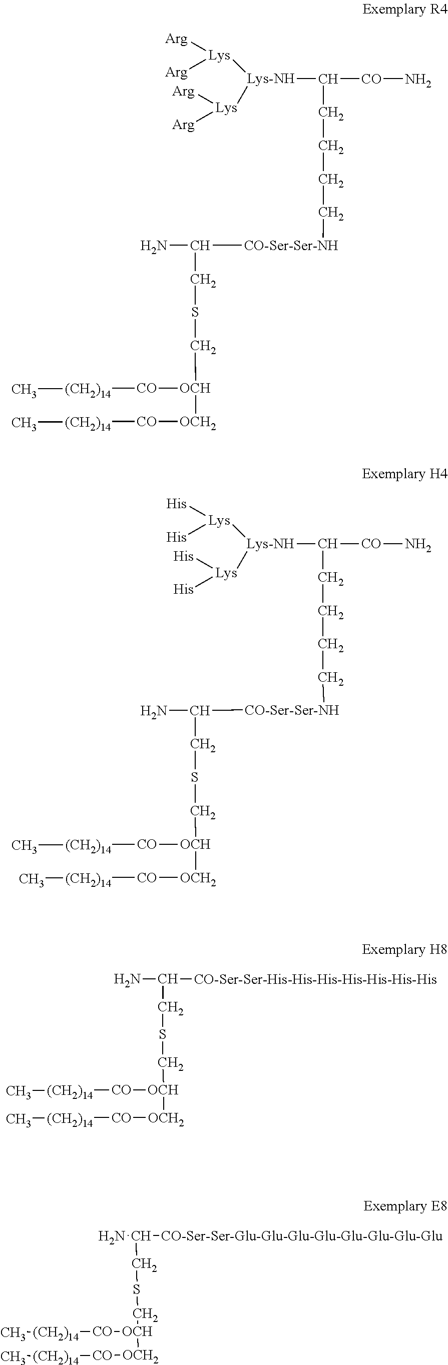

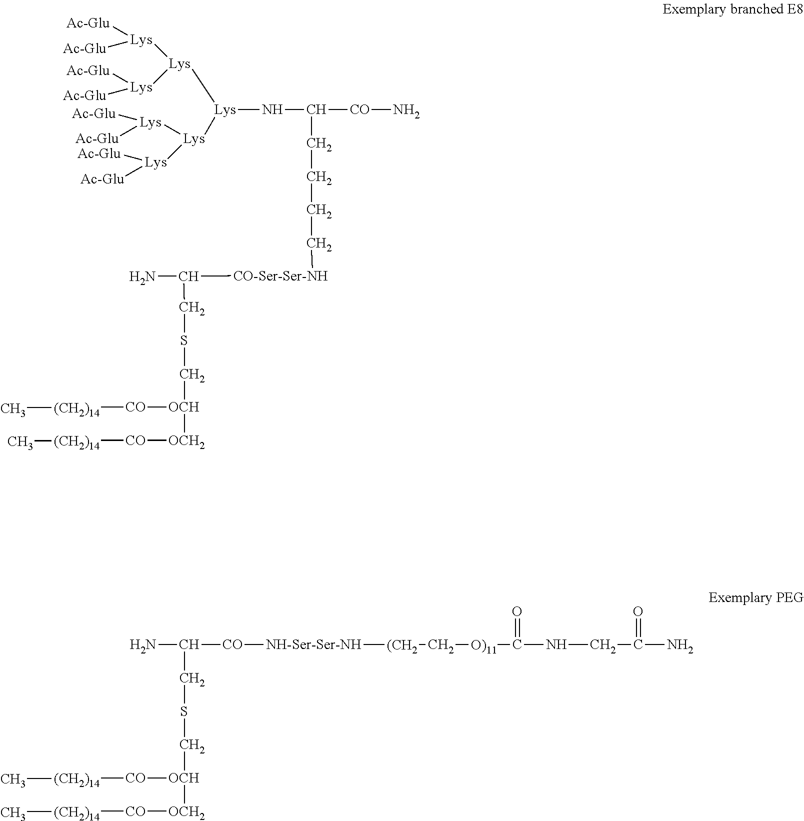

Following are schematic representations of some examples of branched (structures 1-5) and linear (structures 6-8) immunogenic compositions comprising of positively charged (Arginine, R; Lysine, K) or negatively charged (Aspartic acid, D; Glutamic acid, E) amino acids in terminal positions such that their respective electrostatic charges are displayed to the environment. Each immunogenic composition also contains dipalmitoyl-S-glyceryl cysteine (Pam2Cys) which is a ligand for Toll-Like Receptor 2. Two serine residues (Ser) are also incorporated. In the case of construct 2 the peptide structure was assembled in the direction N→C, all other structures shown in the figure were assembled C→N. Positive and negative electrostatic charges are shown as 2−, 2+, 1−, 1+ etc. depending on the size of charge. Ac=acetyl group used to suppress the positive charge of alpha amino groups in the case of N-terminally situated Glutamic acid.

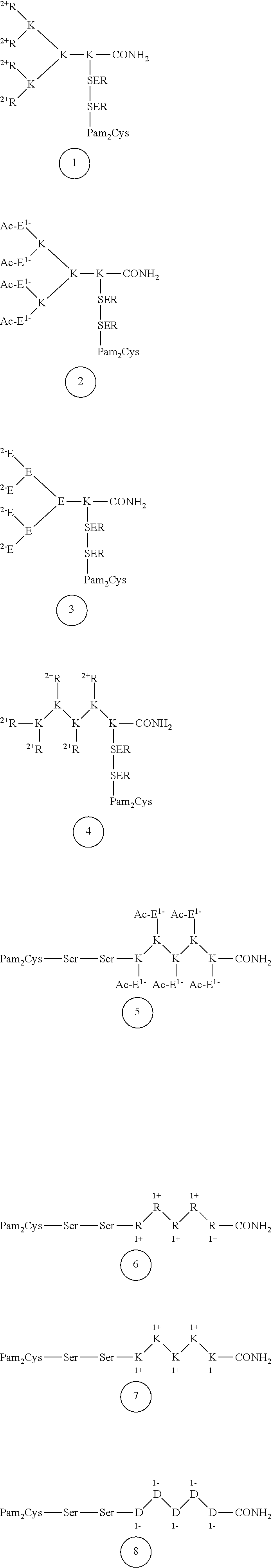

A person skilled in the art will appreciate that the present invention is not limited to the particular exemplified compounds or functional groups that can act as solubilising agents, and that other suitable compounds or functional groups including those that can act as solubilising agents known in the art may be used in accordance with the present invention, such as carbohydrates.

The way in which the one or more compounds or functional group (such as solubilising agents) may be conjugated to a lipid according to the present invention would be well known to a person skilled in the art. For example, conjugation via Fmoc chemistry, through a disulfide or a thioether bridge, or via oxime chemistry is envisaged. In a particular embodiment of the present invention, a soluble form of Pam2Cys was prepared by addition of O—(N-Fmoc-2-aminoethyl)-O′-(2-carboxyethyl)-undecaethyleneglycol (Fmoc-PEOn-OH, Merck Ltd) to Pam2Cys. This resulted in the formation of a PEGylated form of the lipid, Pam2Cys-PEG₁₁ which is then suitable for administration to a subject.

In another form of the invention, the TLR2 moiety comprises a conjugate comprising Pam2Cys conjugated to a pendant R4 form. In a preferred form, pendant-Pam2Cys is conjugated to R4 according to the following structure:

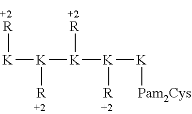

In a preferred form according to any embodiment of the present invention, the TLR2 moiety comprises a conjugate comprising Pam2Cys conjugated to PEG. In a preferred form according to any embodiment of the present invention, the TLR2 moiety comprises a conjugate comprising Pam2Cys conjugated to PEG₁₁ or PEG₁₂. Preferably, the Pam2Cys and PEG₁₁ or PEG₁₂ molecules are separated by at least two serines (PEG₁₁-SS-Pam2Cys or PEG₁₂-SS-Pam2Cys).

As used herein, reference to a TLR2 agonist also includes a pharmaceutically acceptable salt, solvate, polymorph or prodrug thereof.

Additional compounds that comprise a TLR2 agonist that are useful in any aspect of the present invention are described below.

In any aspect, the compound may be a compound of formula (I):

A-Y—B (I)

wherein A comprises or consists of a moiety selected from A1 and A2:

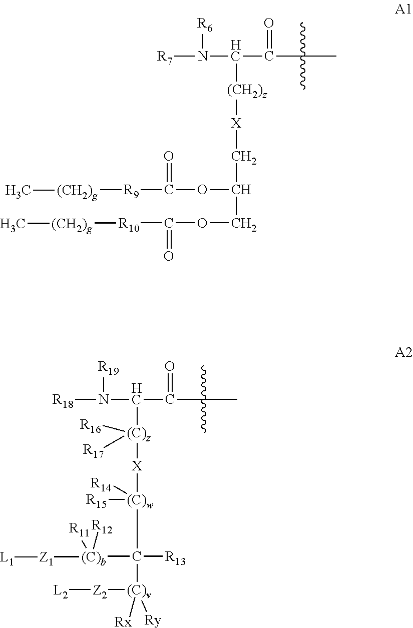

wherein

each z is independently selected from 1 or 2;

each X is independently selected from —S—, —S(═O)— and —S(═O)₂—;

in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and

in moiety A2:
b and w are each independently an integer from 0 to 7 and v is an integer from 0 to 5, such as from 2 to 5, provided that:
   the sum of b, v, and w is at least 3; and
   the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

Y is

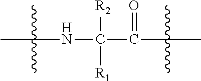

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In any aspect, the compound may be a compound of formula (IA1):

A-Y—B (IA1)

wherein A comprises or consists of moiety A1:

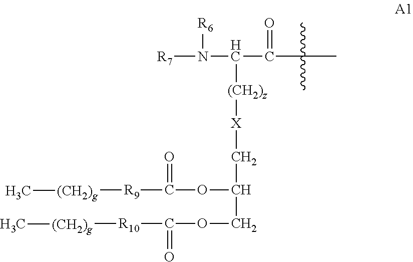

wherein each g is independently 10, 11, 12, 13, 14, 15, 16, 17 or 18;

z is 1 or 2;

X is selected from —S—, —S(═O)— and —S(═O)₂—;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

Y is

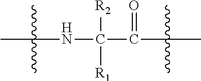

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In some embodiments, g is an integer from 12 to 16.

In some embodiments, g is 14.

In any aspect, the compound may be a compound of formula (IA2):

A-Y—B (IA2)

wherein A comprises or consists of:

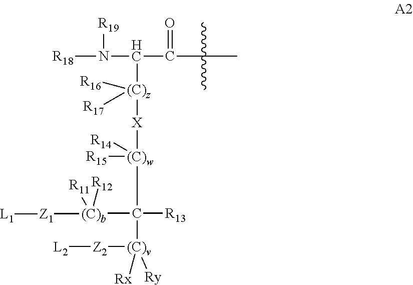

wherein

b and w are each independently an integer from 0 to 7 and v is an integer from 0 to 5, such as from 2 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

Y is

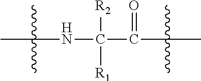

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

and

B comprises or consists of Polyethylene Glycol (PEG),

or a pharmaceutically acceptable salt, solvate or prodrug thereof.

In some embodiments, v is an integer selected from 2, 3, 4 or 5. In some embodiments, v is 2 or 3. In some embodiments, v is 2.

In some embodiments, Rₓ, R_{y}, R₁₁, R₁₂, R₁₃, R₁₄, R₁₅, R₁₆, and R₁₇ are H.

In some embodiments, R and R₁₃ are each H.

In some embodiments, Z₁ and Z₂ are the same and selected from the group consisting of —O—, —NR—, —S—, S(═O), S(═O)₂—, —C(═O)O—, —OC(═O)—, —C(═O)NR—, —NRC(═O)—, —C(═O)S—, —SC(═O)—, OC(═O)O—, NRC(═O)O—, —OC(═O)NR—, and —NRC(═O)NR—.

In some embodiments, Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—.

In some embodiments, w is an integer selected from 1-7. In some embodiments, w is 1.

In some embodiments, b is 0.

In some embodiments, the sum of b and w is from 1 to 7. In these embodiments, b may be an integer selected from 0-7 and w may be an integer selected from 1-7, preferably 1.

In some embodiments, b is 0, w is 1 and v is 2.

In some embodiments, R₁₈ is H.

In some embodiments, R₁₉ is selected from the group consisting of H, C₁-C₆ alkyl, —C(═O) C₁-C₆ alkyl or —C(═O)C₁₁-C₁₉alkyl.

In some embodiments, R₁₉ is selected from H, C₁-C₆ alkyl, —C(═O) C₁-C₆ alkyl, preferably H, C₁-C₄ alkyl, —C(═O) C₁-C₄ alkyl.

In some embodiments, R₁₉ is selected from H and —C(═O)CH₃.

In some embodiments, L₁ and L₂ are independently selected from C₅-C₂₁ aliphatic or C₄-C₂₀ heteroaliphatic. In some embodiments, L₁ and L₂ and independently selected from C₁₀-C₁₈ aliphatic or C₁₀-C₁₈ heteroaliphatic. In some embodiments, L₁ and L₂ are independently selected from C₁₄-alkyl and C₁₅-alkyl.

In some embodiments, X is S.

In some embodiments, X is S(═O).

In some embodiments, X is S(═O)₂.

In some embodiments, R₆ and R₇ are each H.

In some embodiments, R₁₈ and R₁₉ are each H.

In some embodiments, the invention provides a compound of formula (I) wherein:

v is an integer from 2 to 5;

b is 0;

Rₓ, R_{y}, R₁₃, R₁₄, R₁₅, R₁₆, and R₁₇ are H;

Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

w is an integer from 1 to 7;

R₁₉ is selected from the group consisting of H, C₁-C₆ alkyl, —C(═O) C₁-C₆ alkyl or —C(═O)C₁₁-C₁₉alkyl; and

L₁ and L₂ and independently selected from C₁₀-C₁₈ aliphatic or C₁₀-C₁₈ heteroaliphatic.

In some embodiments, the invention provides a compound wherein

v is 2;

b is 0;

w is 1;

the sum of v, b and w is 3;

the sum of b and w is 1;

z is 1;

X is S

Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each H;

R and R₁₃ are each H;

R₁₈ is H;

R₁₉ is selected from the group consisting of H, C₁-C₆ alkyl, —C(═O) C₁-C₆ alkyl or —C(═O)C₁₁-C₁₉alkyl; and

L₁ and L₂ and independently selected from C₁₀-C₁₈ aliphatic or C₁₀-C₁₈ heteroaliphatic.

It will be appreciated that any embodiment of a substituent described herein, including substituents R₁, R₂, R₄, R₅, R₆, R₇, R₉, R₁₀, z, X, g, R₁₁, R₁₂, R₁₃, R₁₄, R₁₅, R₁₆, R₁₇, R₁₃, R₁₉, Rₓ, R_{y}, L₁, L₂, Z₁, Z₂, b, v, w, n, m, p, q, R₃, L, t, k and h, is intended to apply to any instance of that substituent for any compound described herein, including compounds of formulas (I)-(XIX).

In any aspect, the compound may be a compound of formula (II):

A-Y′—B (II)

wherein A comprises or consists of moiety A1 or A2 as defined herein;

Y′ is

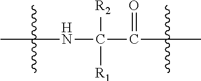

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

and

B comprises or consists of Polyethylene Glycol (PEG),

or a pharmaceutically acceptable salt, solvate or prodrug thereof.

In some embodiments, the compound comprises moiety A1, wherein:

each g is independently 10, 11, 12, 13, 14, 15, 16, 17 or 18;

z is 1;

X is S;

R₆ and R₇ are H;

R₉ and R₁₀ are both a single bond.

In some embodiments, moiety A1 is defined by moiety A1′

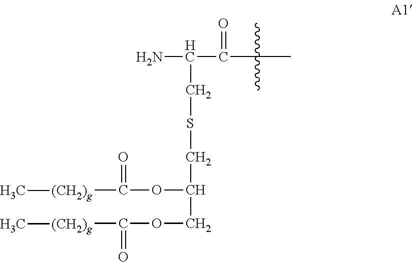

wherein each g is independently 10, 11, 12, 13, 14, 15, 16, 17 or 18.

In any aspect, any of the compounds described herein may be a compound comprising a moiety A selected from A1′ and A2 as defined herein and PEG, wherein the moiety A and PEG are linked by a glycine, serine, homoserine, threonine, phosphoserine, asparagine or glutamine residue, or an ester of a glutamine residue.

In any aspect, the compound may comprise or consist of partial structure A1Y′ or A2Y′:

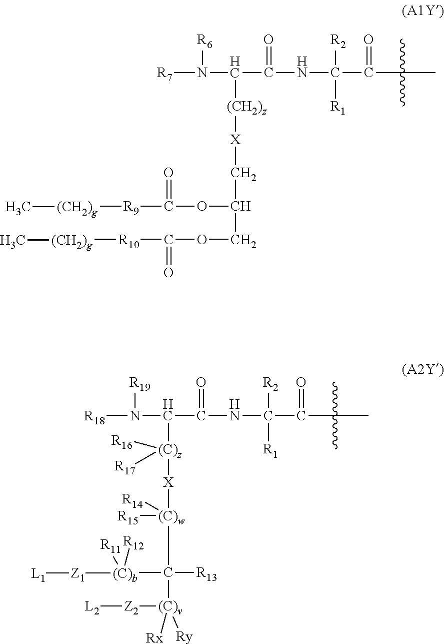

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

b and w are each independently an integer from 0 to 7 and v is an integer from 0 to 5, provided that:

the sum of b, v, and w is at least 3; and

the sum of b and w is from 0 to 7;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A2;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and

A1Y′ or A2Y′ is covalently linked to polyethylene glycol (PEG),

or a pharmaceutically acceptable salt, solvate or prodrug thereof.

In some embodiments, the moiety A and PEG are linked by a serine, homoserine, threonine or phosphoserine residue.

In some embodiments, moiety A and PEG are covalently linked to the glycine, serine, homoserine, threonine, phosphoserine, asparagine or glutamine residue, or an ester of a glutamine residue, through the bond(s) denoted by [symbol] .

In any aspect, the compound may be:

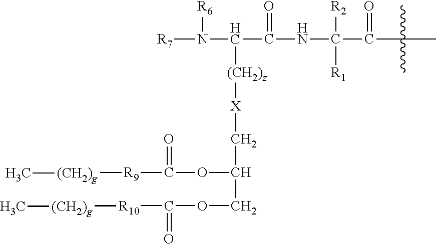

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2; and

X is selected from —S—, —S(═O)— and —S(═O)₂—;

covalently linked to polyethylene glycol (PEG),

or a pharmaceutically acceptable salt, solvate or prodrug thereof.

In some embodiments, the compound may be:

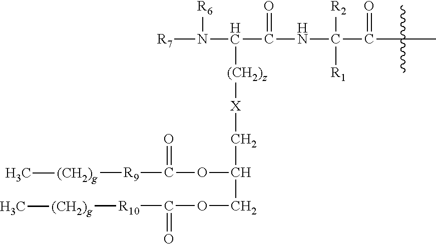

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2; and

X is selected from —S—, —S(═O)— and —S(═O)₂—;

covalently linked to polyethylene glycol (PEG),

or a pharmaceutically acceptable salt, solvate or prodrug thereof.

In some embodiments, the compound may be:

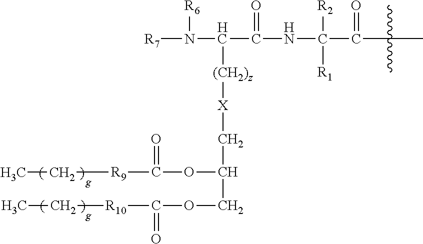

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₈ is selected from the group consisting of H and a straight or branched C₁-C₆ alkyl;

R₉ and R₁₀ are both a single bond;

z is 1; and

X is S;

covalently linked to polyethylene glycol (PEG),

or a pharmaceutically acceptable salt, solvate or prodrug thereof.

In some embodiments, the PEG is covalently linked through the bond denoted by [symbol] .

In some embodiments, the compound may be:

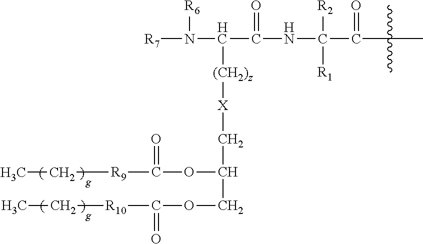

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are H;

R₉ and R₁₀ are both a single bond;

z is 1; and

X is S;

covalently linked to polyethylene glycol (PEG),

or a pharmaceutically acceptable salt, solvate or prodrug thereof.

In some embodiments, the PEG is covalently linked through the bond denoted by [symbol] .

In some embodiments, the compound may be:

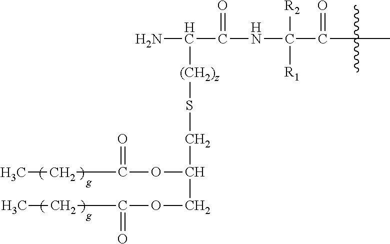

wherein R₁, R₂ and g are as defined herein. salt, solvate or prodrug thereof

In some embodiments, the PEG is covalently linked through the bond denoted by [symbol] .

In some embodiments, the compound may be:

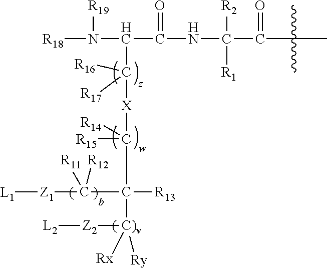

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A2;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

covalently linked to polyethylene glycol (PEG),

or a pharmaceutically acceptable salt, solvate or prodrug thereof.

In some embodiments, the PEG is covalently linked through the bond denoted by [symbol] .

In any aspect, the compound may be a compound of formula (III):

AY-B (III)

wherein

AY comprises or consists of a moiety selected from AY1 and AY2

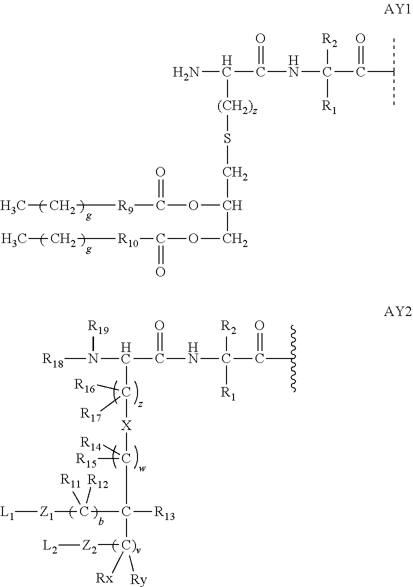

wherein each of R₁, R₂, R₆, R₇, R₈, R₁₀, z, X, g, R₁₁, R₁₂, R₁₃, R₁₄, R₁₅, R₁₆, R₁₇, R₁₈, R₁₉, Rₓ, R_{y}, L₁, L₂, Z₁, Z₂, b, v and w are as defined for the compound of formula (I); and

B comprises or consists of Polyethylene Glycol (PEG).

In any aspect, the compound may be a compound of formula (IV):

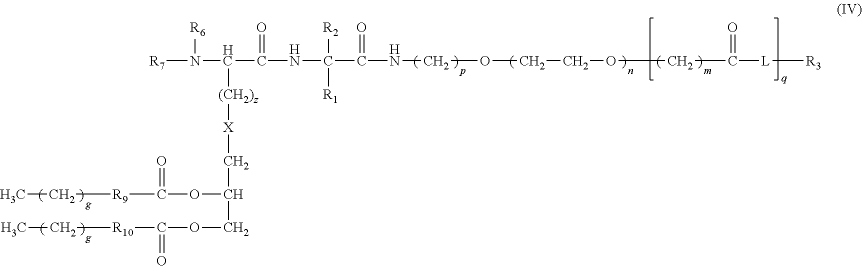

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

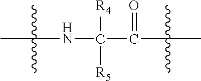

wherein R₄ is H; and

R₅ is the side chain, or second hydrogen of the amino acid

or a pharmaceutically acceptable salt, solvate or prodrug thereof.

In any aspect, the compound may be a compound of formula (V):

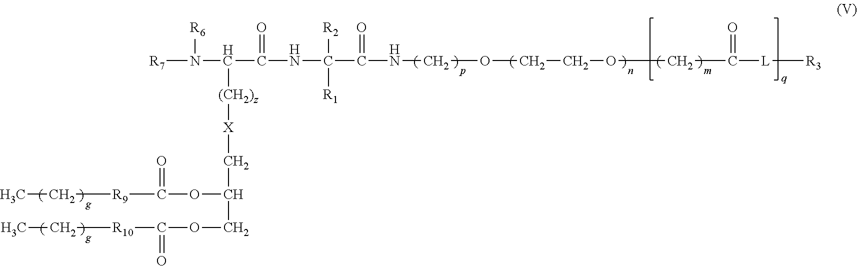

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

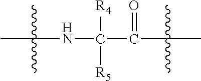

wherein R₄ is H; and

R₅ is the side chain, or second hydrogen of the amino acid

or a pharmaceutically acceptable salt, solvate or prodrug thereof.

In some embodiments, the compound is a compound of formula (IV) or (V) wherein

R₆ and R₇ are H;

R₈ is selected from the group consisting of H and a straight or branched C₁-C₆ alkyl;

R₉ and R₁₀ are both a single bond;

z is 1; and

X is S.

In some embodiments, the compound of any one of formulas (I)-(V) may be a compound of formula (VI):

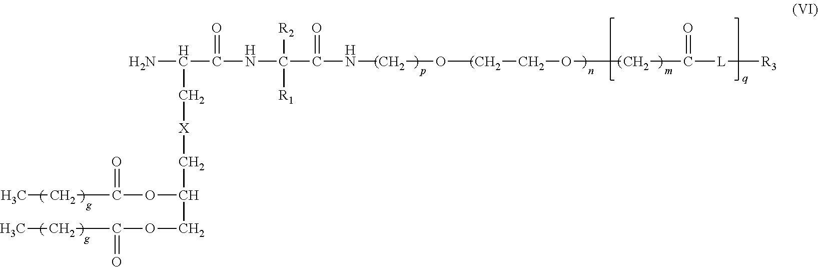

wherein

n is 3 to 100;

m is 1, 2, 3 or 4;

each g is independently 10, 11, 12, 13, 14, 15, 16, 17 or 18;

p is 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

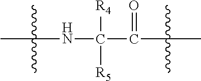

wherein R₄ is H; and

R₅ is the side chain, or second hydrogen of the amino acid

or a pharmaceutically acceptable salt, solvate or prodrug thereof.

In any aspect, the compound may be a compound of formula (VII):

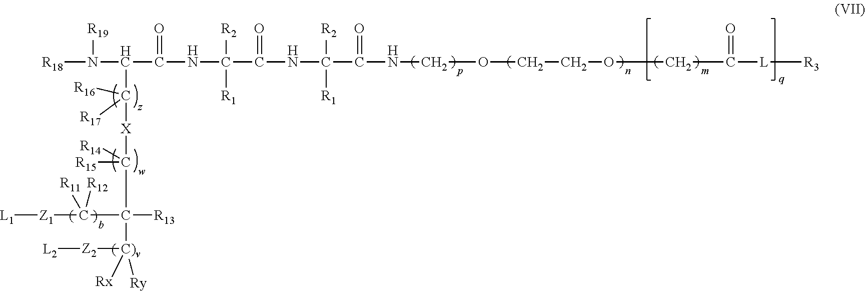

wherein

n is 3 to 100;

m is 1, 2, 3 or 4;

p is 2, 3 or 4;

q is null or 1;

R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;

R₈ is selected from the group consisting of H and a straight or branched C₁-C₆ alkyl;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

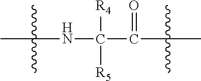

wherein R₄ is H; and

R₅ is the side chain, or second hydrogen of the amino acid;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A₂;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

or a pharmaceutically acceptable salt, solvate or prodrug thereof.

In any aspect, the compound may be a compound of formula (VIII):

A-Y—NH—(CH₂)ₚ—O—(CH₂—CH₂—O)ₙ—[(CH₂)ₘ—CO-L-]_{q}R₃ (VIII)

wherein

A is a moiety selected from A1 and A2 as defined herein

Y is

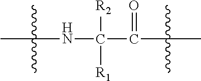

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

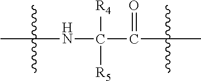

wherein R₄ is H; and

R₅ is the side chain, or second hydrogen of the amino acid,

or a pharmaceutically acceptable salt, solvate or prodrug thereof.

In any aspect, the compound may be a compound of formula (IX):

A1-Y—NH—(CH₂)ₚ—O—(CH₂—CH₂—O)ₙ—[(CH₂)ₘ—CO-L-]_{q}R₃ (IX)

wherein

A1 is represented by moiety A1 as defined for formula (I)

Y is

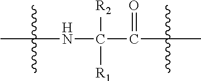

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

n is 3 to 100;

m is 1, 2, 3 or 4;

each g is independently 10, 11, 12, 13, 14, 15, 16, 17 or 18;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

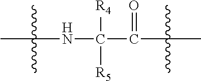

wherein R₄ is H; and

R₅ is the side chain, or second hydrogen of the amino acid,

or a pharmaceutically acceptable salt, solvate or prodrug thereof.

In some embodiments, the compound is a compound of formula (VIII) or (IX), wherein

R₆ and R₇ are H;

R₉ and R₁₀ are both a single bond;

z is 1;

X is S.

In any aspect, the compound may be a compound of formula (X):

Pam2Cys-Y—NH—(CH₂)ₚ—O—(CH₂—CH₂—O)ₙ—[(CH₂)ₘ—CO-L-]_{q}R₃ (X)

wherein

Pam2Cys has the structure:

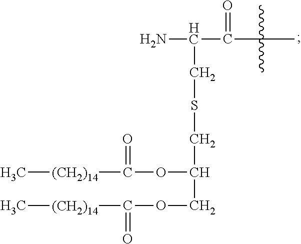

Y is:

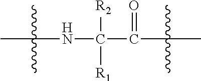

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₅ alkyl;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

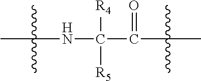

wherein R₄ is H; and

R₅ is the side chain, or second hydrogen of the amino acid,

or a pharmaceutically acceptable salt, solvate or prodrug thereof.

In any aspect, the compound may be a compound of formula (XI):

Pam2Cys-Y—NH—(CH₂)ₚ—O—(CH₂—CH₂—O)ₙ—[(CH₂)ₘ—CO-L-]_{q}R₃ (XI)

wherein

Pam2Cys has the structure:

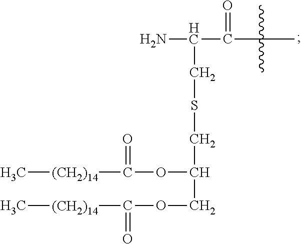

Y is:

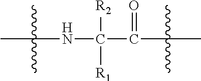

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen and wherein R₁ and R₂ are not both H;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

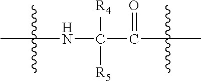

wherein R₄ is H; and

R₅ is the side chain, or second hydrogen of the amino acid,

or a pharmaceutically acceptable salt, solvate or prodrug thereof.

In any aspect, the compound may be a compound of formula (XII):

Pam2Cys-Y—NH—(CH₂)ₚ—O—(CH₂—CH₂—O)ₙ—[(CH₂)ₘ—CO-L-]_{q}R₃ (XII)

wherein

Pam2Cys has the structure:

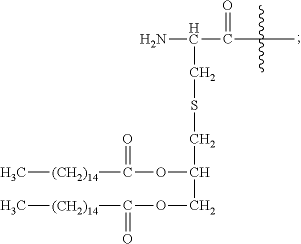

Y is:

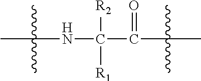

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

n is 3 to 100;

m is 1, 2, 3 or 4;

p is 2, 3 or 4;

q is null or 1;

wherein when q=1, R₃ is H, —NH₂ or —OH;

wherein when q=0, R₃ is H;

L is null or consists of 1 to 10 units, wherein each unit is a natural alpha amino acid or derived from a natural alpha amino acid, and has the formula:

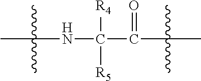

wherein R₄ is H; and

R₅ is the side chain, or second hydrogen of the amino acid,

or a pharmaceutically acceptable salt, solvate or prodrug thereof.

In any aspect, the compound may be a compound of formula (XIII):

Pam2Cys-Ser-NH—(CH₂)ₚ—O—(CH₂—CH₂—O)ₙ—[(CH₂)ₘ—CO-L-]_{q}R₃ (XIII)

wherein

Pam2Cys-Ser has the structure:

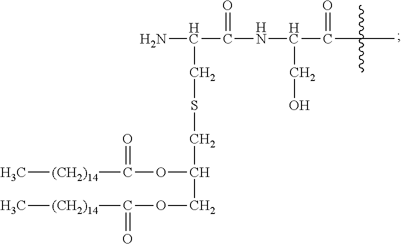

n is 3 to 100;

m is 1, 2, 3 or 4;

p is 2, 3 or 4;

q is null or 1;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

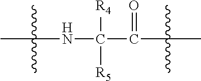

wherein R₄ is H; and

R₅ is the side chain, or second hydrogen of the amino acid,

or a pharmaceutically acceptable salt, solvate or prodrug thereof.

In one embodiment, the compound has the formula (XIV):

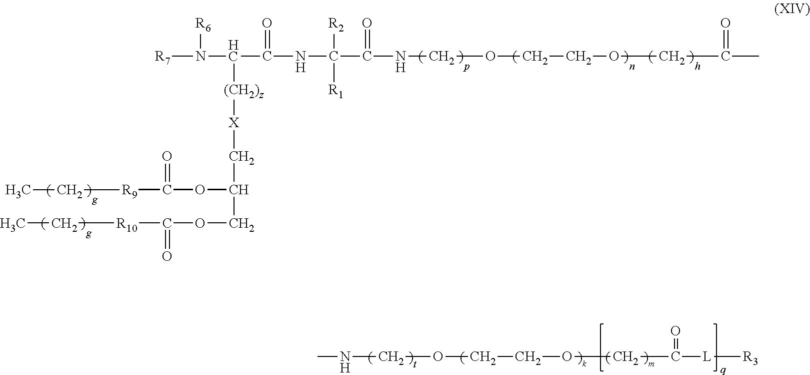

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are independently selected from the group consisting of H, a straight or branched C₁-C₄ alkyl, and —C(═O)CH₃;

R₈ is selected from the group consisting of H and a straight or branched C₁-C₆ alkyl;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

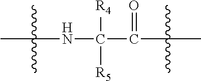

wherein R₄ is H; and

R₅ is the side chain, or second hydrogen of the amino acid,

or a pharmaceutically acceptable salt, solvate or prodrug thereof.

In one embodiment, the compound has the formula (XV):

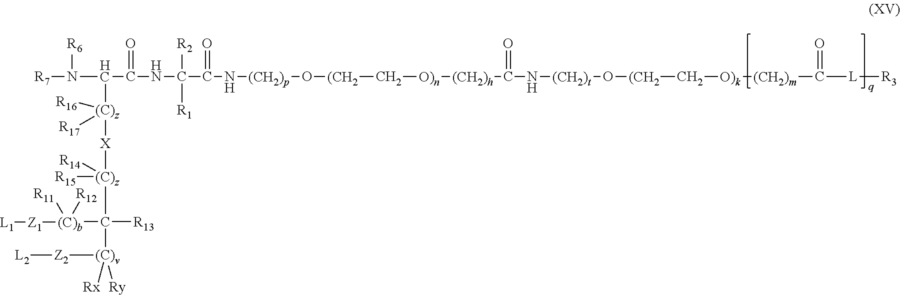

wherein

n is 3 to 100;

k is 3 to 100;

m is 1, 2, 3 or 4;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

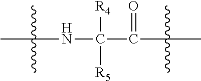

wherein R₄ is H; and

R₅ is the side chain, or second hydrogen of the amino acid;

b and w are each independently an integer from 0 to 7 and v is an integer from 0 to 5, provided that:

the sum of b, v, and w is at least 3; and

the sum of b and w is from 0 to 7;

z is 1 or 2;

X is selected from —S—, —S(═O)— and —S(═O)₂—;

Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;

R₁₁, R₁₂, Rₓ, R_{y}, R₁₄, R₁₅, R₁₆, and R₁₇ at each instance of b, v, w, and z are each independently H or C₁-C₆ aliphatic;

R, R₁₃ and R₁₈ are each independently H or C₁-C₆ aliphatic;

R₁₉ is H, C₁-C₆ aliphatic, an amino protecting group, L₃-C(═O)—, or A2;

L₁ and L₂ are each independently C₅-C₂₁ aliphatic or C₄-C₂₀ heteroaliphatic;

L₃ is C₁-C₂₁ aliphatic or C₂-C₂₀ heteroaliphatic;

A₂ is an amino acid or a peptide;

wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;

or a pharmaceutically acceptable salt, solvate or prodrug thereof.

In any aspect, the compound may be a compound of formula (XVI):

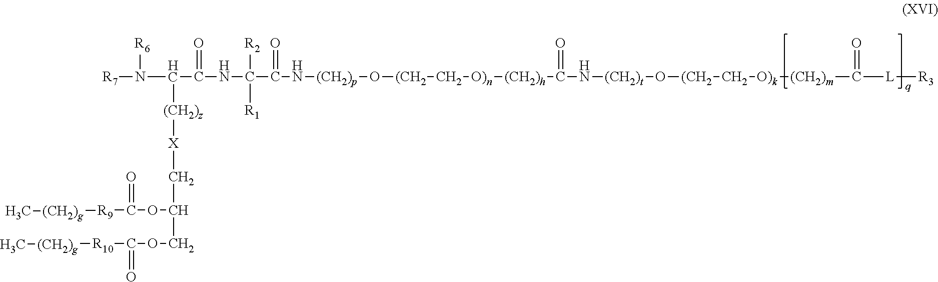

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are independently selected from the group consisting of H, a straight or branched C₁-C₄ alkyl, and —C(═O)CH₃;

R₉ and R₁₀ are independently selected from the group consisting of —NH—, —O— or a single bond;

z is 1 or 2;

X is selected from —S—, —S(═O)— and —S(═O)₂—;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

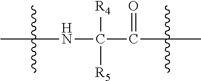

wherein R₄ is H; and

R₅ is the side chain, or second hydrogen of the amino acid,

or a pharmaceutically acceptable salt, solvate or prodrug thereof.

In any aspect, the compound may be a compound of formula (XVII):

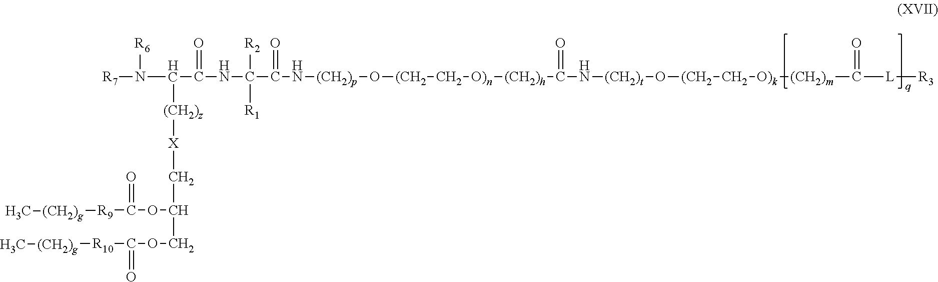

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₆ and R₇ are H;

R₈ is selected from the group consisting of H and a straight or branched C₁-C₆ alkyl;

R₉ and R₁₀ are both a single bond;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

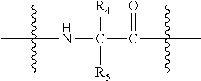

wherein R₄ is H; and

R₅ is the side chain, or second hydrogen of the amino acid,

or a pharmaceutically acceptable salt, solvate or prodrug thereof.

In any aspect, the compound may be a compound of formula (XVIII):

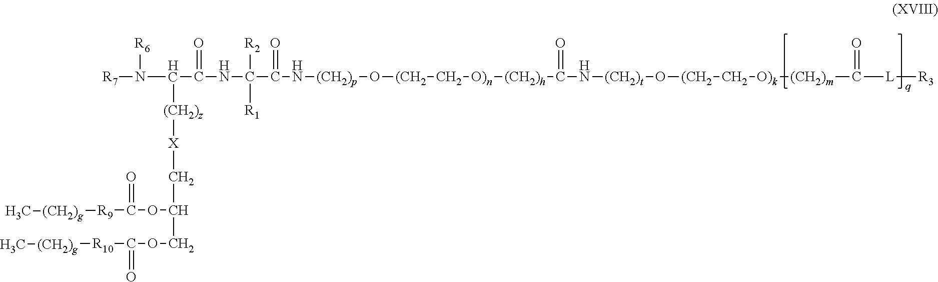

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

R₆ and R₇ are H;

R₉ and R₁₀ are both a single bond;

z is 1;

X is S;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

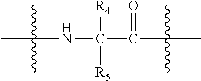

wherein R₄ is H; and

R₅ is the side chain, or second hydrogen of the amino acid,

or a pharmaceutically acceptable salt, solvate or prodrug thereof.

In any aspect, the compound may be a compound of formula (XIX):

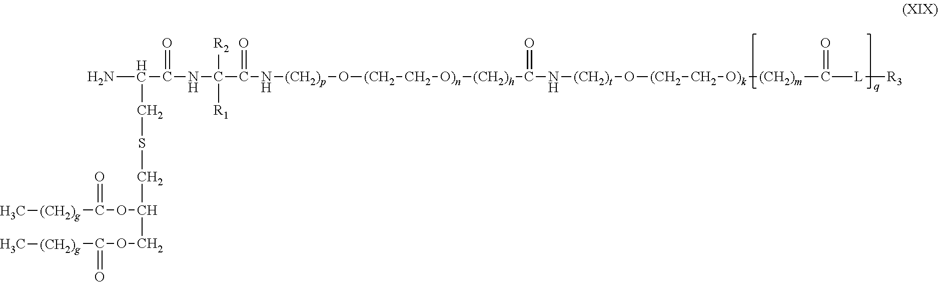

wherein

n is 3 to 100;

k is 3 to 100;

m is 1, 2, 3 or 4;

each g is independently 10, 11, 12, 13, 14, 15, 16, 17 or 18;

p is 2, 3 or 4;

t is 2, 3 or 4;

h is 1, 2, 3 or 4;

q is null or 1;

R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;

wherein when q=1, R₃ is —NH₂ or —OH;

wherein when q=0, R₃ is H;

L is null or consists of 1 to 10 units, wherein each unit is a natural alpha amino acid or derived from a natural alpha amino acid, and has the formula:

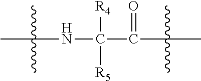

wherein R₄ is H; and

R₅ is the side chain, or second hydrogen of the amino acid,

or a pharmaceutically acceptable salt, solvate or prodrug thereof.

In some embodiments, any compound disclosed herein (including a compound of any one of formulas (I)-(XIX)) that comprises polyethylene glycol (PEG) may comprise the PEG in the form of a substituted PEG.

In some embodiments, the substituted PEG is represented by partial formula B-I:

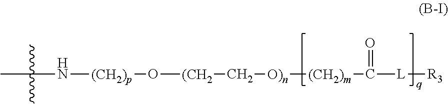

wherein

n is 3 to 100;

m is 1, 2, 3 or 4;

p is 2, 3 or 4;

q is null or 1;

R₃ is H, —NH₂ or —OH, wherein when q is null, R₃ is H and when q is 1, R₃ is —NH₂ or —OH;

L is null or consists of 1 to 10 units, wherein each unit is a natural alpha amino acid or derived from a natural alpha amino acid, and has the formula:

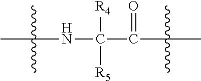

wherein R₄ is H; and

R₅ is the side chain, or second hydrogen of the amino acid.

In some embodiments, the substituted PEG is represented by partial formula B-II:

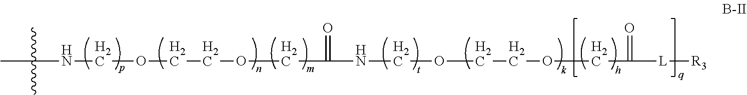

wherein

p is 2, 3 or 4;

n is 3 to 100;

m is 1, 2, 3 or 4;

t is 2, 3 or 4;

k is 3 to 100;

h is 1, 2, 3 or 4;

q is null or 1;

wherein when q is 1, R₃ is —NH₂ or —OH;

wherein when q is null, R₃ is H;

L is null or consists of 1 to 10 units, wherein each unit is a natural alpha amino acid or derived from a natural alpha amino acid, and has the formula:

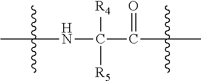

wherein R₄ is H; and

R₅ is the side chain, or second hydrogen of the amino acid.

In some embodiments of the substituted PEG of formula B-I or B-II, q is 1.

In some embodiments of the substituted PEG of formula B-I or B-II, n may be from 10 to 14, such as 11, or from 24 to 30, such as 27.

In some embodiments of the substituted PEG of formula B-I or B-II, m is from 1 to 3, such as 2.

In some embodiments of the substituted PEG of formula B-I or B-II, when q is 1, R₃ is —NH₂.

In some embodiments of the substituted PEG of formula B-I or B-II, L is a natural alpha amino acid residue.

Compounds described herein may exist in and be isolated in optically active and racemic forms. As would be understood by a person skilled in the art, the present invention is intended to encompass any racemic, optically active or stereoisomeric form, or mixtures thereof, of compounds of the invention which possess the useful properties described herein. It is well known in the art how to prepare such forms (for example, by resolution of racemic mixtures by recrystallization, by synthesis from optically-active starting materials, by chiral synthesis, or by chiral chromatographic separation). In some embodiments, a composition may comprise a compound in an enantiomerically or diastereomerically enriched form. For example, the compound may have an enantiomeric excess (ee) or a diastereomeric excess (de) of at least about 1%, 5%, 10%, 20%, 30%, 40%, 50%, 60%, 70%, 80%, 90%, 95%, 96%, 97%, 98%, 99% or more than 99%. In some embodiments, the compound may be enriched by at least about 1%, 5%, 10%, 20%, 30%, 40%, 50%, 60%, 70%, 80%, 90%, 95%, 96%, 97%, 98%, 99% or more than 99% at any stereocentre of the compound.

In any aspect, the compound may comprise a chiral centre around the following chiral centre (shown at *):

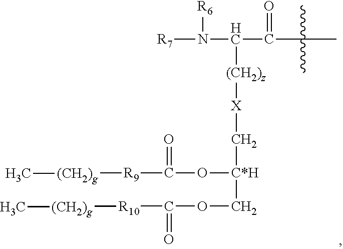

wherein the chiral centre is in the R configuration. A compound in this form may also be referred to as an R-Pam2 analogue diastereomer of a compound of the invention as described herein. This may be depicted as:

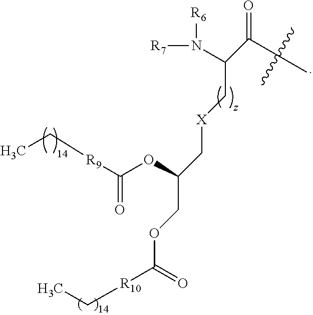

In any aspect, the compound may comprise a chiral centre in the 2,3-bis(palmitoyloxy)propyl moiety of Pam2Cys (shown at *):

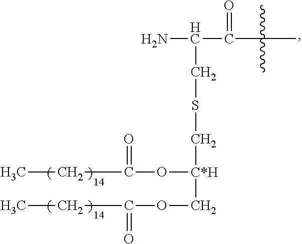

wherein the chiral centre is in the R configuration. A compound in this form may also be referred to as an R-Pam2 diastereomer of a compound of the invention as described herein. This may be depicted as:

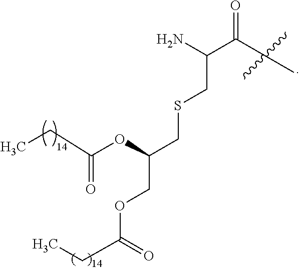

In any aspect, the compound may comprise a chiral centre around the following chiral centre (shown at *):

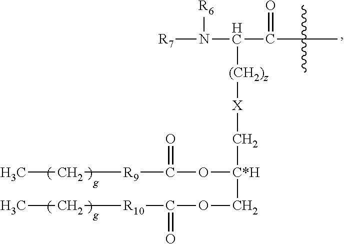

wherein the chiral centre is in the S configuration. A compound in this form may also be referred to as an S-Pam2 analogue diastereomer of a compound of the invention as described herein. This may be depicted as:

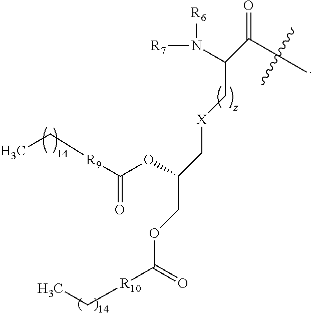

In any aspect, the compound comprises a chiral centre in the 2,3-bis(palmitoyloxy)propyl moiety of Pam2Cys (shown at *):

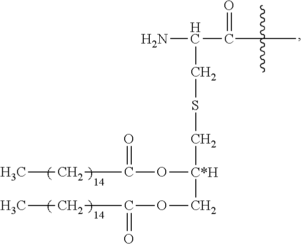

wherein the chiral centre is in the S configuration. A compound in this form may also be referred to as an S-Pam2 diastereomer of a compound of the invention as described herein. This may be depicted as:

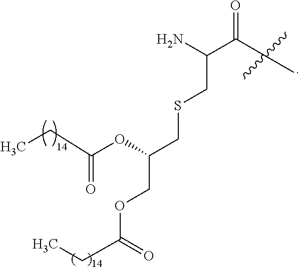

In any aspect, the compound comprises a chiral centre around the following chiral centre (shown at *):

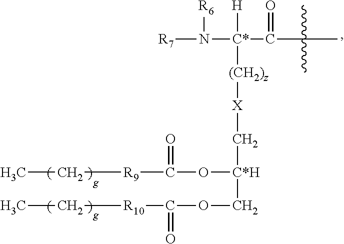

wherein the chiral centre is in the L configuration. A compound in this form may also be referred to as an L-Cys analogue diastereomer of Pam2Cys of a compound as described herein.

This may be depicted as:

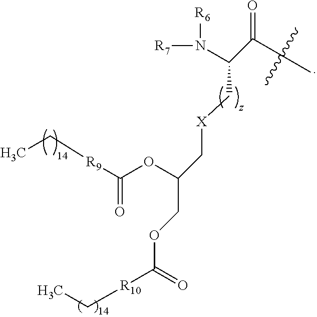

In any aspect, the compound comprises a chiral centre in the cysteine residue of Pam2Cys (shown at *):

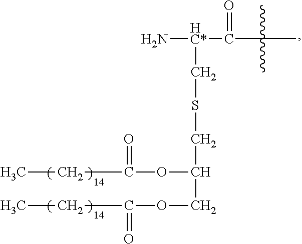

wherein the chiral centre is in the L configuration. A compound in this form may also be referred to as an L-Cys diastereomer of Pam2Cys of a compound as described herein. This may be depicted as:

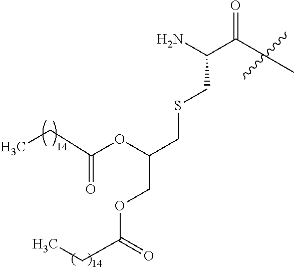

Other stereocentres in these compounds may be racemic or independently enriched in either the R or S configuration.

In any aspect, the compound comprises a chiral centre in moiety A1 around the following chiral centre (shown at *):

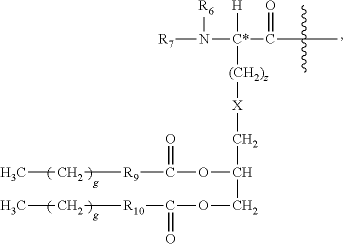

wherein the chiral centre is in the D configuration. A compound in this form may also be referred to as an D-Cys analogue diastereomer of Pam2Cys of a compound described herein.

This may be depicted as:

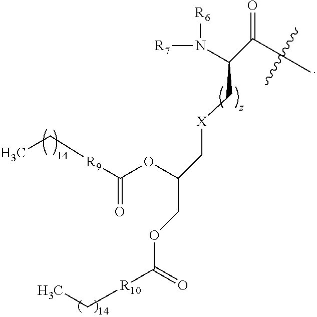

Other stereocentres in these compounds may be racemic or independently enriched in either the R or S configuration.

In any aspect, the compound comprises a chiral centre in the cysteine residue of Pam2Cys (shown at *):

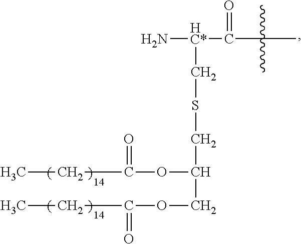

wherein the chiral centre is in the D configuration. A compound in this form may also be referred to as an D-Cys diastereomer of Pam2Cys of a compound described herein. This may be depicted as:

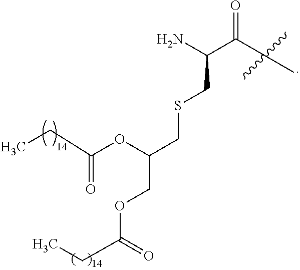

Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

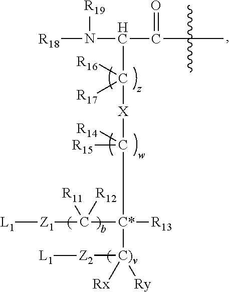

wherein the chiral centre is in the R configuration. In some embodiments, this stereoisomer of the compound may be depicted as:

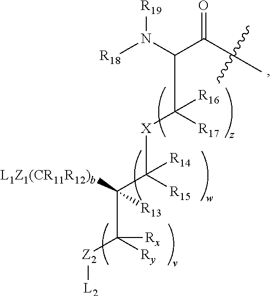

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound of Formula (I) and w is 1. Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

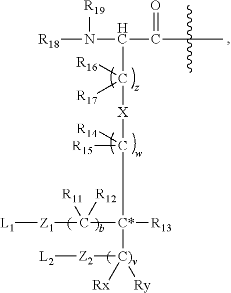

wherein the chiral centre is in the S configuration. In some embodiments, moiety A of this stereoisomer of the compound may be depicted as:

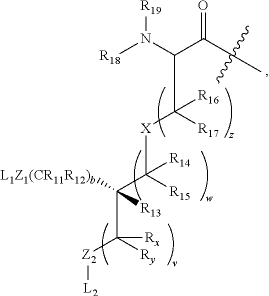

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

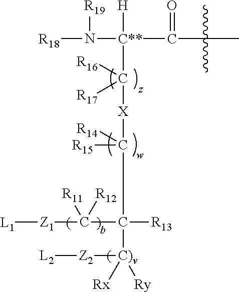

wherein the chiral centre is in the L configuration. A compound in this form may also be referred to as an L-Cys analogue stereoisomer of a compound of the invention. In some embodiments, this stereoisomer of the compound may be depicted as:

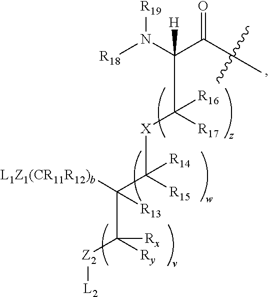

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

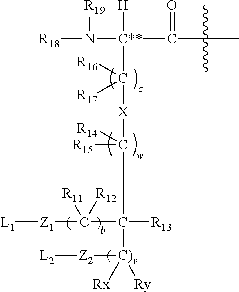

wherein the chiral centre is in the D configuration. A compound in this form may also be referred to as a D-Cys analogue stereoisomer of a compound of the invention. In some embodiments, moiety A of this stereoisomer of the compound may be depicted as:

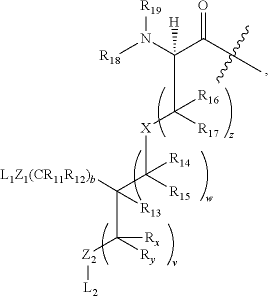

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound or Formula (I) and w is 1. Other stereocentres in these compounds may be racemic or independently enriched in either the R or S configuration.

In any aspect, the compound comprises a chiral centre in the Y moiety of the compound (shown at *):

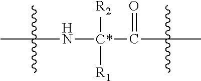

wherein the chiral centre is in the L-configuration. A compound in this form may also be referred to as an L-Y diastereomer of a compound of the invention described herein.

In any aspect, the compound comprises a chiral centre in the Y moiety of the compound (shown at *):

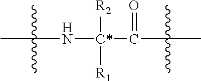

wherein the chiral centre is in the D-configuration. A compound in this form may also be

In any aspect, compositions comprising a compound of the invention (including a compound of any one of formulas (I)-(XIX)) or a pharmaceutically acceptable salt, solvate or prodrug thereof, and a pharmaceutically acceptable carrier, diluent or excipient may be used in a method or use of the invention.

In some embodiments, the compound as described herein is the R diastereomer around the chiral centre of the 2,3-bis(palmitoyloxy)propyl moiety of the compound.

In some embodiments, the compound as described herein is the S diastereomer around the chiral centre of the 2,3-bis(palmitoyloxy)propyl moiety of the compound.

In any aspect, a composition as described herein comprises a compound that is the R diastereomer around the chiral centre of the 2,3-bis(palmitoyloxy)propyl moiety of the compound.

In any aspect, a composition comprises a compound that is the S diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the R diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the S diastereomer around the chiral centre of the 2,3-bis(palmitoyloxy)propyl moiety of the compound (for example moiety A1).

In any aspect, the compound as described herein is the L diastereomer around the chiral centre of the cysteine analogue residue of the Pam2Cys analogue moiety compound (for example moiety Y).

In any aspect, the compound as described herein is the L diastereomer around the chiral centre of the cysteine residue of the Pam2Cys moiety compound (for example moiety Y).

In any aspect, the compound as described herein is the D diastereomer around the chiral centre of the cysteine analogue residue of the Pam2Cys analogue moiety compound (for example moiety Y).

In any aspect, the compound as described herein is the D diastereomer around the chiral centre of the cysteine residue of the Pam2Cys moiety of the compound (for example moiety Y).

In any aspect, a composition as described herein comprises a compound that is the L diastereomer around the chiral centre of the cysteine analogue residue of the Pam2Cys analogue moiety of the compound (for example moiety Y).

In any aspect, a composition as described herein comprises a compound that is the L diastereomer around the chiral centre of the cysteine residue of the Pam2Cys moiety of the compound (for example moiety Y).

In any aspect, a composition as described herein comprises a compound that is the D diastereomer around the chiral centre of the cysteine analogue residue of the Pam2Cys analogue moiety of the compound (for example moiety Y).

In any aspect, a composition as described herein comprises a compound that is the D diastereomer around the chiral centre of the cysteine residue of the Pam2Cys moiety of the compound (for example moiety Y).

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine residue of the Pam2Cys moiety of the compound.

In any aspect 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine residue of the Pam2Cys moiety of the compound.

In any aspect, the compound of the invention as described herein is the L diastereomer around the chiral centre of the Y moiety.

In any aspect, the compound as described herein is the D diastereomer around the chiral centre of the Y moiety.

In any aspect, a composition as described herein comprises a compound that is the L diastereomer around the chiral centre of the Y moiety.

In any aspect, a composition as described herein comprises a compound that is the D diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the Y moiety.

The compounds of formulas (I)-(XIX) described herein may demonstrate substantial stability in solution. This solution stability may be observed by storing solutions of the compounds under ambient storage conditions (eg at 25° C.) or under accelerated degradation stability (eg at 40° C.) for at least about 14 days.

In any aspect, any of the compounds described herein may be administered in the form of a pharmaceutically acceptable salt.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound of the invention as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of any of compounds of Formulas (I)-(XIX). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds described herein, including the compounds of formulas (I)-(XIX), or other structure as depicted herein.

The general chemical terms used in the formulae herein have their usual meaning.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include linear or branched, heteroalkyl, heteroalkenyl, and heteroalkynyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C═CH, —C═CH₃, —CH₂C═CH₃, and —C═CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. In some embodiments, the heteroalkyl is saturated.

Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” is intended to include non-aromatic ring systems containing 3 or more ring atoms, of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 ring atoms. Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl.

The term “heteroaryl” is intended to include aromatic ring systems containing 5 or more ring atoms, of which, one or more is a heteroatom. In some embodiments, the heteroatom is nitrogen, oxygen, or sulfur. In some embodiments, heteroaryl groups include mono-, bi- and tricyclic ring systems having from 5 to 16, from 5 to 14, from 5 to 12, from 5 to 10, from 5 to 8, or from 5 to 6 ring atoms. Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.

The term “halo” or “halogen” is intended to include F, Cl, Br, and I.

The term “heteroatom” is intended to include oxygen, nitrogen, sulfur, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent(s) are attached is not exceeded, and that the substitution results in a stable compound. In some embodiments, optional substituents in the compounds described herein include but are not limited to halo, CN, NO₂, OH, NH₂, NHR₁₀₀, NR₁₀₀R₂₀₀, C₁₋₆haloalkyl, C₁₋₆haloalkoxy, C(O)NH₂, C(O)NHR₁₀₀, C(O)NR₁₀₀R₂₀₀, SO₂R₁₀₀, OR₁₀₀, SR₁₀₀, S(O)R₁₀₀, C(O)R₁₀₀, and C₁₋₆aliphatic; wherein R₁₀₀ and R₂₀₀ are each independently C₁₋₆aliphatic, for example C₁₋₆alkyl.

Where a protecting group (PG) is referred to, a person skilled in the art would readily understand what type of protecting group would be suitable.

The term “amine protecting group” as used herein is intended to mean a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl). In some embodiments, the amine protecting groups for the purposes described herein include (but are not limited to) tert-butyloxycarbonyl (t-Boc) and 9H-fluoren-9-ylmethoxycarbonyl (Fmoc).

The term “carboxyl protecting group” as used herein is intended to mean a group that is capable of being readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “carboxamide protecting group” as used herein is intended to mean a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

As used herein, ‘Ser’ refers to the amino acid serine and ‘Cys’ refers to the amino acid cysteine.

As used herein, ‘PEG’ refers to the polymer compound polyethylene glycol. Unless otherwise defined, reference to ‘PEG’ includes any length polymer of ethylene oxide. Reference to PEG also includes substituted PEG. In some embodiments, substituted PEG may be defined by formulas B-I or B-II as described herein.

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Stimulators of the Immune System

In an aspect of the invention, there is provided a method for treating, preventing or minimising the progression of cancer comprising administering a TLR2 agonist and an immunostimulant.

A skilled person will understand that the terms “immunostimulant,” “immunostimulatory,” or “stimulating/inducing an immune response,” and grammatical equivalents thereof, as used herein refers to inducing, increasing, enhancing, or otherwise providing a beneficial effect with respect to an immune response.

“Immune stimulation” or “immunostimulatory” or “stimulating/inducing an immune response” refers to a direct or indirect response of an immune system cell or component to any treatment described herein. Such a responses can be measured by any known means in the art including activation, proliferation or differentiation of immune system cells (B cells, T cells, dendritic cells, APCs, macrophages, NK cells, NKT cells etc.), up-regulated or down-regulated expression of markers, cytokine, interferon, IgM and IgG release in the serum, and mixed cellular infiltrates in various organs.

Immunostimulants may include specific immunostimulants and non-specific immunostimulants. Specific immunostimulants provide antigenic specificity in immune response, such as vaccines or any antigen. Non-specific immunostimulants act irrespective of antigenic specificity to augment immune response of other antigen or stimulate components of the immune system without antigenic specificity, such as adjuvants and non-specific immunostimulators.

Such immunostimulants may assist the immune system via any one or more different ways, including by priming and boosting the immune system via stimulation of antigen-presenting cells, T-cells, or innate cells; by reducing immunosuppression in the tumour environment by regulating inhibitory pathways; and/or by enhancing the adaptive or innate immune responses. Thus, the stimulation of the immune system may be of innate immune system cells, or of the acquired immune system cells. An immunostimulant may inhibit the immunosuppressive effect induced by a cancer cell or an antigen presenting cell.

As used herein, an immunostimulant may be a molecule that does not directly stimulate the immune system but maybe repolarise immune cells or inhibit immune-suppression. Alternatively, the immunostimulant may directly stimulate or active the immune system.

Innate immunity refers to those immune responses that occur rapidly after infection or development of cancer. They are initiated without prior sensitization to the pathogen or malignant cell, are not antigen specific and are mediated directly by phagocytic cells such as macrophages, cytotoxic cells such as natural killer (NK) cells and antigen presenting cells such as dendritic cells (DCs) as well as indirectly by the cytokines produced by these cells. Adaptive immunity or cellular immunity refers to those responses that require some time to develop after initial infection or cancer development and involves an education of immune cells, resulting in the development of a highly specific, highly potent and long-lived response. This is mediated by cytotoxic T-lymphocytes (CTLs), helper T-lymphocytes and antibody-producing B-lymphocytes. Along these lines, adaptive immune responses are classified as either cellular (those mediated by CTLs) or humoral (antibody mediated responses), with helper T-lymphocytes facilitating both responses. Together, the rapid innate immune response functions to control early spread of the disease and facilitates development of adaptive immune responses while the highly potent, specific and long-lived adaptive response serves to clear the disease as well as to protect against recurrence.

The present invention contemplates the use of an immunostimulant in combination with any TLR2 agonist described herein for the treatment, prevention or minimisation of progression of cancer. Suitable immunostimulants that may be used in accordance with the methods described herein include:
cellular immunotherapies (cytotoxic cellular immunotherapies or adoptive cellular therapies);
oncolytic viruses;
cancer vaccines;
T-cell engagers; and
bispecific T-cell engagers.

Specific examples of suitable immunostimulants include Blincyto (blinatumomab), Oncotice (Bacillus Calmette-Guerin [BCG] [strain Tice] vaccine), BCG (BCG [strain Rivm] vaccine), ImmuCyst (ImmuCyst), Pacis (BCG [strain Montreal] vaccine), Provenge (sipuleucel-T), DCVax-L (DCVax-L), Oncorine (human adenovirus type 5 [recombinant]), and Imlygic (talimogene laherparepvec).

Suitable immunostimulants for use in the methods of the present invention also include an activator of a costimulatory molecule or an inhibitor of an immune checkpoint molecule. Therefore, an immunostimulant may be an immune checkpoint inhibitor, a costimulatory molecule agonist, or an immune activating agent. Examples of checkpoint inhibitors useful in the present invention are described herein. Inhibition of an inhibitory molecule can be performed by inhibition at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand, or an antibody or antigen-binding fragment thereof that binds to the inhibitory molecule.

Costimulatory molecules may be any one of the following costimulatory molecule is chosen from an agonist of one or more of OX40, CD2, CD27, CDS, ICAM-1, LFA-1, (CD11a/CD18), ICOS (CD278), 4-IBB (CD137), GITR, CD30, CD40, BAFFR, HVEM, CD7, LIGHT, NKG2C, SLAMF7, NKp80, CD160, B7-H3 or CD83 ligand. An agonist of any one of these costimulatory molecules is also contemplated as an immunostimulant.

Checkpoint Inhibitors

A “checkpoint inhibitor” inactivates a protein in an inhibitory checkpoint pathway of an immune response.

In the context of cancer, checkpoint inhibitors regulate the immune system by blocking proteins that stop the immune system from attacking cancer cells. In particular, they control how detection-evading cancer cells and T-cells interact so that T-cells can recognize tumour cells and mount an appropriate immune response against them. Non-limiting examples of checkpoint inhibitors that may be used in accordance with the methods described herein include inhibitors that target PD-1 (programmed cell death protein 1), CTLA-4 (cytotoxic T lymphocyte associated protein 4) and PD-L1 (programmed death ligand 1). A skilled person will understand that CTLA-4 and PD-1 are found on T cells and that PD-L1 is expressed on cancer cells. Other non-limiting examples include PD-L2, TIM3, LAG3, CEACAM (e.g., CEACAM-1, CEACAM-3 and/or CEACAM-5), VISTA, BTLA, TIGIT, LAIR1, CD160, 2B4, CD80, CD86, B7-H3 (CD276), B7-H4 (VTCN1), HVEM (TNFRSF14 or CD107), KIR, A2aR, MHC class I, MHC class II, GAL9, adenosine, and TGF-beta.

Other immune checkpoints include Indoleamine 2,3-dioxygenase (IDO) and CSF-R1. Inhibitors of those proteins are also contemplated as immune checkpoint inhibitors for use in the invention.

“Programmed Death-1 (PD-1)” refers to an immunoinhibitory receptor belonging to the CD28 family. PD-1 is expressed predominantly on activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

Upon binding of PD-1 to programmed cell death ligand 1 (PD-L1), an immune reaction is turned off so as to prevent T-cells from damaging or killing the cell. In the context of cancer, cancer cells can be covered with PD-L1 proteins to camouflage themselves as healthy cells thus avoiding an immune response. Programmed Death Ligand-1 (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (HPD-L1), variants, isoforms, and species homologs of hPD-L1, and analogs having at least one common epitope with hPD-L1. The complete hPD-L1 sequence can be found under GenBank Accession No. Q9NZQ7.

Cytotoxic T-Lymphocyte Antigen-4 (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

Any of the checkpoint inhibitors described herein may be administered in the form of an antibody. An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FRI, CDR1, FR2, CDR2, FR3, CDR3, FR4. The variable regions of the heavy and light chains contain a binding domain that interacts with an antigen. The constant regions of the Abs can mediate the binding of the immunoglobulin to host tissues or factors, including various cells of the immune system (e.g., effector cells) and the first component (C1q) of the classical complement system.

The term “antibody” includes, by way of example monoclonal and polyclonal Abs; chimeric and humanized Abs; human or nonhuman Abs; wholly synthetic Abs; and single chain Abs. A nonhuman Ab can be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 can, however, have cross-reactivity to other antigens, such as PD-1 molecules from different species. Moreover, an isolated Ab can be substantially free of other cellular material and/or chemicals. The term “monoclonal antibody” (mAb) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. mAbs can be produced by hybridoma, recombinant, transgenic or other techniques known to those skilled in the art.

A “human” antibody (huMAb) refers to an Ab having variable regions in which both the framework and CDR regions are derived from human germline immune globulin sequences. Furthermore, if the Ab contains a constant region, the constant region also is derived from human germline immunoglobulin sequences. The human Abs of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-PD-1 Ab binds specifically to PD-1 and an anti-CTLA-4 Ab binds specifically to CTLA-4.

An “antigen-binding portion” of an Ab (also called an “antigen-binding fragment”) refers to one or more fragments of an Ab that retain the ability to bind specifically to the antigen bound by the whole Ab.

Examples of immune checkpoints and antibody inhibitors that target those checkpoints include anti-CTLA-4 (e.g., Ipilimumab, Tremelimumab, KAHR-102), anti-TIM3 (e.g., F38-2E2. ENUM005), anti-LAG3 (e.g., BMS-986016, IMP701. IMP321, C9B7W), anti-KIR (e.g., Lirilumab, IPH2101, IPH4102), anti-PD-1 (e.g., Nivolumab, Pidilizumab, Pembrolizumab, BMS-936559, atezolizumab, Lambrolizumab, MK-3475. AMP-224, AMP-514, STI-A1110, TSR-042), anti-PD-L1 (e.g., KY-1003 (EP20120194977), MCLA-145, atezolizumab. BMS-936559, MEDI-4736, MSB0010718C, AUR-012, STI-A1010. PCT/US2001/020964, MPDL3280A, AMP-224, Dapirolizumab pegol (CDP-7657), MEDI-4920), anti-CD73 (e.g., AR-42 (OSU-HDAC42, HDAC-42, AR42, AR 42, OSU-HDAC 42, OSU-HDAC-42, NSC D736012, HDAC-42, HDAC 42, HDAC42, NSCD736012, NSC-D736012), MEDI-9447), anti-B7-H3 (e.g., MGA271, DS-5573a, 8H9), anti-CD47 (e.g., CC-90002, TTI-621, VLST-007), anti-BTLA, anti-VISTA, anti-A2aR, anti-B7-1, anti-B7-H4, anti-CD52 (such as alemtuzumab), anti-IL-10, anti-IL-35, anti-TGF-β (such as Fresolumimab), anti-CSF1R (e.g., FPA008), anti-NKG2A (e.g., monalizumab), anti-MICA (e.g., IPH43), and anti-CD39.

Anti-PD-1 Antibodies and Anti-PD-L1 Antibodies

Examples of suitable PD-1 inhibitors that may be used in accordance with the invention include Keytruda (pembrolizumab), Opdivo (nivolumab), AGEN 2034, BGB-A317, BI-754091, CBT-501 (genolimzumab), MEDI0680, MGA012, PDR001, PF-06801591, REGN2810 (SAR439684), and TSR-042 or those that are disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493.

Nivolumab (also known as “Opdivo®”; formerly designated 5C4, BMS-936558, MDX-1106, or ONO4538) is a fully human IgG4 (S228P) PD-1 immune check point inhibitor Ab that selectively prevents interaction with PD-1 ligands (PD-L1 and PD-L2), thereby blocking the down-regulation of antitumor T-cell functions (U.S. Pat. No. 8,008,449).

Pembrolizumab (also known as “Keytruda®”, lambrolizumab, and MK-3475) is a humanized monoclonal IgG4 antibody directed against human cell surface receptor PD-1 (programmed death-1 or programmed cell death-1). Pembrolizumab is described for example, in U.S. Pat. Nos. 8,354,509 and 8,900,587). Pembrolizumab has been approved by the FDA for the treatment of relapsed or refractory melanoma.

Other suitable PD-1 inhibitors include Libtayo (cemiplimab), Blincyto (blinatumomab), Dostarlimab, Spartalizumab, Cetrelimab, Pidilizumab and BI-754091.

Anti-PD-1 Abs suitable for use in the disclosed methods or compositions are Abs that bind to PD-1 with high specificity and affinity, block the binding of PD-L1 and or PD-L2, and inhibit the immunosuppressive effect of the PD-1 signalling pathway. In any of the compositions or methods disclosed herein, an anti-PD-1 antibody includes an antigen-binding portion or fragment that binds to the PD-1 receptor and exhibits the functional properties similar to those of whole Abs in inhibiting ligand binding and upregulating the immune system.

In certain embodiments, an anti-PD-1 antibody used in the methods can be replaced with another PD-1 or anti-PD-L1 antagonist. For example, because an anti-PD-L1 antibody prevents interaction between PD-1 and PD-L1, thereby exerting similar effects to the signaling pathway of PD-1, an anti-PD-L1 antibody can replace the use of an anti-PD-1 antibody in the methods disclosed herein. In any embodiment, suitable PD-L1 inhibitors include Imfinzi (durvalumab or MED14736), Tecentriq (atezolizumab or MPDL3280A), Bavencio (avelumab; MSB0010718C), MS-936559 (12A4 or MDX-1105) and CX-072.

Anti-CTLA-4 Antibodies

Anti-CTLA-4 antibodies of the instant invention bind to human CTLA-4 so as to disrupt the interaction of CTLA-4 with a human B7 receptor. It will be understood that because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Suitable CTLA-4 inhibitors that may be used in accordance with the invention include Yervoy (ipilimumab), Tremelimumab and AGEN 1884 or those disclosed in U.S. Pat. Nos. 6,984,720 and 7,605,238. Ipilimumab is a fully human, IgG1 monoclonal Ab that blocks the binding of CTLA-4 to its B7 ligands, thereby stimulating T cell activation. Tremelimumab is human IgG2 monoclonal anti-CTLA-4 antibody. Another is Blincyto (blinatumomab) which is a Bispecific CD19-directed CD3 T-cell engager.

Administration and Dosage

In an embodiment of the invention, therapeutically effective amounts of a TLR2 agonist and a checkpoint inhibitor are administered to the subject.

Administering refers to the physical introduction of a composition comprising a therapeutic agent to a subject, using any of the various methods and delivery systems known to those skilled in the art including those described herein. Pharmaceutical compositions may be formulated from compounds of the invention as described herein for any appropriate route of administration. Typically, in addition to the therapeutic agent (eg a TLR2 agonist and/or immunostimulant), a pharmaceutical composition comprises a pharmaceutically acceptable excipient, carrier and/or diluent. Examples of suitable components for inclusion in a pharmaceutical composition are described in Martindale—The Extra Pharmacopoeia (Pharmaceutical Press, London 1993) and Martin (ed.), Remington's Pharmaceutical Sciences.

Suitable routes of administration for implementing the defined methods include oral, intravenous, intramuscular, topical, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion, as well as in vivo electroporation. Administering can also be performed, for example, once, a plurality of times, and/or over one or more extended periods.

Any composition described herein may be formulated for administration to the respiratory tract, in other words via a respiratory route. Where administration to all or part of the respiratory tract is contemplated, a skilled person will understand that this includes administration intranasally or via inhalation, in particular for administration to the lung. The composition as described herein may be formulated for intranasal administration, including dry powder, sprays, mists, or aerosols.

Suitable formulations, wherein the carrier is a liquid, for administration, as for example, a nasal spray or as nasal drops, include aqueous or oily solutions of the active ingredient. Alternatively, the composition may be a dry powder and administered to the respiratory tract only as defined herein.

The selection of appropriate carriers depends upon the particular type of administration that is contemplated. For administration via the respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the invention, various devices are available in the art for the generation of drops, droplets and sprays. For example, a TLR2 agonist and/or immunostimulant, or composition described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The tear secretions of the eye drain from the orbit into the nasal passages, thus, if desirable, a suitable pharmaceutically acceptable ophthalmic solution can be readily provided by the ordinary artisan as a carrier for the compound or composition described herein to be delivered and can be administered to the orbit of the eye in the form of eye drops to provide for both ophthalmic and intranasal administration.

In one embodiment, a premeasured unit dosage dispenser that includes a dropper or spray device containing a solution or suspension for delivery as drops or as a spray is prepared containing one or more doses of the drug to be administered. The invention also includes a kit containing one or more unit dehydrated doses of compound, together with any required salts and/or buffer agents, preservatives, colorants and the like, ready for preparation of a solution or suspension by the addition of a suitable amount of water. The water may be sterile or nonsterile, although sterile water is generally preferred.

The phrase ‘therapeutically effective amount’ or ‘effective amount’ generally refers to an amount of a TLR2 agonist and/or checkpoint inhibitor, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

For instance, for the treatment of tumours, a therapeutically effective amount of the compounds or compositions described herein can inhibit tumour growth by at least about 10%, by at least about 20%, by at least about 30%, by at least about 40%, by at least about 50%, by at least about 60%, by at least about 70%, by at least about 80%, or by at least about 90% or more, relative to untreated subjects. Alternatively, the treatments described herein may cause complete regression of the tumour mass. In other embodiments of the invention, tumour regression can be observed and continue for a period of at least about 10 days, at least about 20 days, at least about 30 days, at least about 40 days, at least about 50 days or at least about 60 days, at least about 70 days, at least about 80 days, at least about 90 days, at least about 100 days or longer.

A therapeutically effective amount of a drug may also include a “preventative” or “prophylactically effective amount,” which is any amount of the TLR2 agonist and/or checkpoint inhibitor administered to a subject at risk of developing a cancer (eg a subject having a pre-malignant condition) or of suffering a recurrence of cancer, that inhibits the development or recurrence of the cancer. In certain embodiments, the prophylactically effective amount prevents the development or recurrence of the cancer entirely. “Inhibiting” or “preventing” the development or recurrence of a cancer means either lessening the likelihood of the cancer's development or recurrence, or preventing the development or recurrence of the cancer entirely.

The exact amount of the therapeutically effective amount required will vary from subject to subject, depending on the species, age and general condition of the subject, mode of administration and the like. Thus, it may not be possible to specify an exact therapeutically effective amount. However, an appropriate therapeutically effective amount in any individual case may be determined by one of ordinary skill in the art using routine experimentation. In one aspect, the dose administered to a subject is any therapeutically effective amount that reduces symptoms associated with the cancer as a result of any one of a reduction in the number of cancer cells; a reduction in the tumour size; an inhibition (i.e., slow to some extent and preferably stop) of cancer cell infiltration into peripheral organs; an inhibition (i.e., slow to some extent and preferably stop) of tumour metastasis; an inhibition, to some extent, of tumour growth; or relieving, to some extent, of one or more of the symptoms associated with the cancer. Additionally or alternatively, the therapeutically effective amount may lead to increased survival of the subject.

In some embodiments, a therapeutically effective amount of a TLR2 agonist for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

Typically, a therapeutically effective dosage is formulated to contain a concentration (by weight) of at least about 0.1% up to about 50% or more, and all combinations and sub-combinations of ranges therein. The compositions can be formulated to contain one or more compounds, or a pharmaceutically acceptable salt, polymorph or prodrug thereof in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

For administration of a checkpoint inhibitor including PD-1, PD-L1 or CTLA-4 inhibitors, the dosage can range from about 0.01 to about 20 mg/kg, about 0.1 to about 10 mg/kg, about 0.1 to about 5 mg/kg, about 1 to about 5 mg/kg, about 2 to about 5 mg/g, about 7.5 to about 12.5 mg/kg, or about 0.1 to about 30 mg/kg of the subject's body weight. For example, dosages can be about 0.1, about 0.3, about 1, about 2, about 3, about 5 or about 10 mg/kg body weight, or, about 0.3, about 1, about 2, about 3, or about 5 mg/kg body weight. The dosing schedule is typically designed to achieve exposures that result in sustained receptor occupancy (RO) based on typical pharmacokinetic properties of an Ab. An exemplary treatment regime entails administration about once per week, about once every 2 weeks, about once every 3 weeks, about once every 4 weeks, about once a month, about once every 3-6 months or longer. In certain embodiments, a checkpoint inhibitor is administered to the subject about once every 2 weeks. In other embodiments, the Ab is administered about once every 3 weeks. The dosage and scheduling can change during a course of treatment. For example, a dosing schedule for anti-PD-1 therapy can comprise administering the Ab: (i) about every 2 weeks in about 6-week cycles; (ii) about every 4 weeks for about six dosages, then about every three months; (iii) about every 3 weeks; (iv) about 3-about 10 mg/kg once followed by about 1 mg/kg every about 2-3 weeks. Considering that an IgG4 Ab typically has a half-life of 2-3 weeks, a dosage regimen for an anti-PD-1 Ab of the invention comprises about 0.3-1 about 0 mg/kg body weight, 1-5 mg/kg body weight, or about 1-about 3 mg/kg body weight via intravenous administration, with the Ab being given every about 14-21 days in up to about 6-week or about 12-week cycles until complete response or confirmed progressive disease.

In some embodiments, the checkpoint inhibitor and/or TLR2 agonist treatment disclosed herein, is continued for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years.

It will be understood, that the specific dose level for any particular patient will depend upon a variety of factors including the activity of the specific compound employed, the age, body weight, general health, sex, diet, time of administration, route of administration, and rate of excretion, drug combination i.e. other drugs being used to treat the patient), and the severity of the particular disorder undergoing therapy.

The terms “treatment” or “treating” of a subject includes the application or administration of a compound of the invention to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, minimising or preventing the progression of cancer means treating the subject so as to prevent or delay the recurrence or metastasis of a tumour, or to prevent growth of an existing tumour. Minimising or preventing the progression of cancer includes preventing or delaying the recurrence of cancer, or preventing growth of an existing tumour, following treatment of cancer. The recurrence that is being prevented includes a recurrence for example, in the tumour bed, following surgical excision. Alternatively, recurrence includes metastasis of the cancer in another part of the body. The terms “preventing recurrence” and “preventing relapse” as used herein, are interchangeable.

The present invention also includes methods of preventing the development of cancer in an individual. For example, the individual for whom prevention of cancer is required may be considered to be at risk of developing cancer, but does not yet have detectable cancer. An individual at risk of the development of cancer may be an individual with a family history of cancer, and/or an individual for whom genetic testing or other testing indicates a high risk or high likelihood of the development of cancer. The individual may have cancer stem cells but does not yet have any detectable tumours. It will be understood that methods of preventing the development of cancer include methods of delaying the onset of cancer in a subject.

The terms “subject” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

Cancer

The term “cancer” will be understood to include benign, pre-cancerous, pre-neoplastic or non-metastatic tumours or metastatic tumours.

In some embodiments, the type of cancer to be treated includes those having a benign, pre-cancerous, pre-neoplastic or non-metastatic tumour. A benign tumour will be understood to not be a malignant tumour and to not invade nearby tissue or spread to other parts of the body. Similarly non-metastatic cancer will be understood to not invade nearby tissue or spread to other parts of the body. “Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

Pre-neoplastic, neoplastic and metastatic cancers are particular examples to which the methods of the invention may be applied. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers. endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. B-cell, mixed cell, null-cell, T-cell, T-cell chronic, HTLV-II associated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

The existence of, improvement in, treatment of, or minimisation of progression of cancer may be determined by any clinically or biochemically relevant method as described herein or known in the art. A positive response to treatment or a minimisation of progression of a cancer may be determined by any method known in the art and may include the determination of:
a reduction in the number of cancer cells;
a reduction in the tumour size;
an inhibition (i.e., slow to some extent and preferably stop) of cancer cell infiltration into peripheral organs;
an inhibition (i.e., slow to some extent and preferably stop) of tumour metastasis;
a reduction or complete prevention of tumour metastasis following removal of the primary tumour;
an inhibition, to some extent, of tumour growth;
relieving, to some extent, of one or more of the symptoms associated with the cancer; and/or
increased survival of the subject.

The determination of any of the above may be considered to be a positive response to a TLR agonist and/or a checkpoint inhibitor described herein.

In contrast, a negative response or a lack of response of a cancer to a treatment including any TLR agonist and/or a checkpoint inhibitor described herein may be determined by any method known in the art and may include the determination of:
no change or an increase in the number of cancer cells;
no change or an increase in the tumour size;
no change, continued or increased cancer cell infiltration into peripheral organs;
no change, continued or increased tumour metastasis;
no change or increased tumour metastasis, despite removal of the primary tumour;
no change or an increase of tumour growth;
no change or an increases of one or more of the symptoms associated with the cancer; and/or
no change or decreased survival of the subject.

In an embodiment of the invention, the subject may have previously received treatment. In an embodiment the previous treatment is a checkpoint inhibitor which may be in the form of an inhibitor of PD-1, PD-L1 or CTLA-4. In a preferred embodiment, the checkpoint inhibitor is in the form of an antibody.

The subject who has received the treatment for cancer may be in partial or complete remission. In other words, the subject, having received a treatment for cancer, as described above, may have a 10%, 20%, 30%, 40%, 50%, 60%, 70%, 80%, 90% or greater reduction in the measurable parameters of tumour growth as may be found on physical examination, radiologic study, or by biomarker levels from a blood or urine test. Alternatively, where the subject is in complete remission, there is a complete disappearance of all detectable manifestations of disease, such that the subject does not have any detectable signs of cancer. The subject may have substantially undetectable signs of cancer. A cancer that is “substantially undetectable” generally refers to a circumstance where therapy has depleted the size, volume or other physical measure of a cancer so that using relevant standard detection techniques such as in vivo imaging, the cancer, as a consequence of the therapy, is not clearly detectable.

The objective or outcome of treatment with TLR2 agonist and/or checkpoint inhibitor may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying cancer progression. In one embodiment, the method is particularly useful for extending survival of the subject, including overall survival as well as progression free survival. It will be understood that overall survival is the length of time from either the date of diagnosis or the start of treatment of a cancer, that patients diagnosed with the cancer are still alive. It will be understood that progression free survival is the length of time during and after the treatment of a cancer that a patient lives with the disease but it does not get worse.

Survival analysis can be performed using well known techniques in the art including the Kaplan-Meier method. The Kaplan-Meier method estimates the survival function from life-time data. In medical research, it can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In one embodiment, the method is particularly useful for providing a complete response to therapy whereby all signs of cancer in response to treatment have disappeared. This does not always mean the cancer has been cured. In one embodiment, the method is particularly useful for providing a partial response to therapy whereby there has been a decrease in the size of one or more tumours or lesions, or in the extent of cancer in the body, in response to treatment.

Kits

In another embodiment there is provided a kit or article of manufacture comprising a TLR2 agonist and/or a checkpoint inhibitor as described herein, a pharmaceutically acceptable salt, diluent or excipient and/or pharmaceutical composition as described above. Further, the kit may comprise instructions for use in any method or use of the invention as described herein.

In other embodiments there is provided a kit for use in a therapeutic and/or prophylactic application mentioned above, the kit comprising:
a container holding a therapeutic composition in the form of a TLR2 agonist and/or a checkpoint inhibitor as described herein, or a pharmaceutically acceptable salt, diluent or excipient or pharmaceutical composition;
a label or package insert with instructions for use.

In certain embodiments the kit may contain one or more further active principles or ingredients for treatment of cancer.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat a cancer described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a cancer or prevent progression of a cancer described herein.

As used herein, the following compounds are described in the table below and specific structures shown elsewhere herein and contemplated in any method or use of the invention.

| | Compound |
|---|---|
| Compound Structure | name |
| 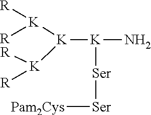 | 001 |
| 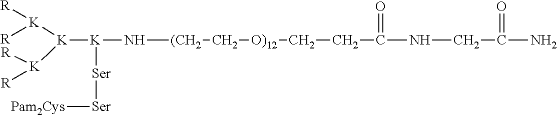 | 002 |
| 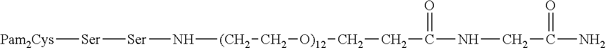 | 003 |
| Pam₂Cys—Ser—Ser—Lys—Lys—Lys—Lys | 004 |
| Pam₂Cys—Ser—Lys—Lys—Lys—Lys | 005 |
| 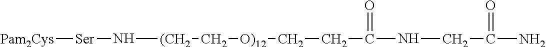 | A101 / compound 1 |
| 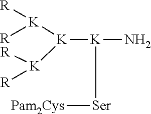 | 007 |
| 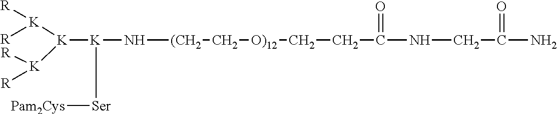 | 008 |
| 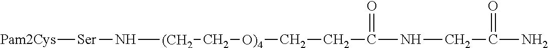 | 009 |
| 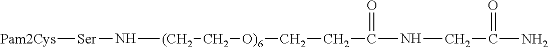 | 010 |
| 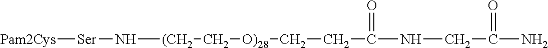 | A105 |
| 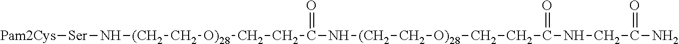 | A106 |
| 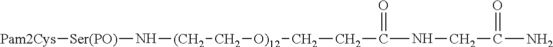 | A104 |
| 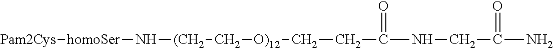 | A103 |
| 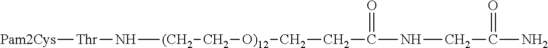 | A102 |
| 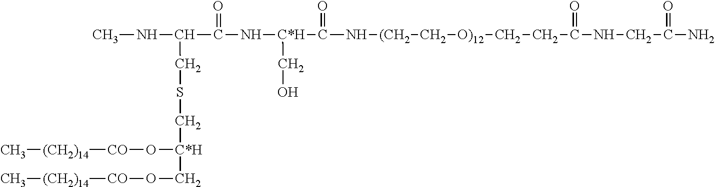 | A109 |
| 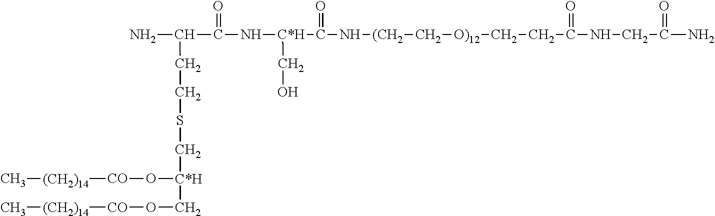 | A110 |
| 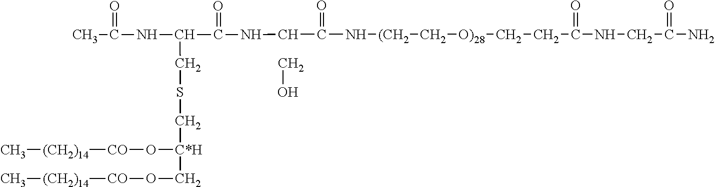 | A111 |
| 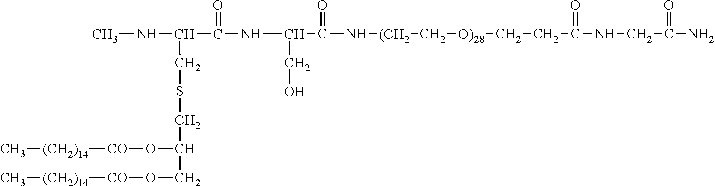 | A112 |
| 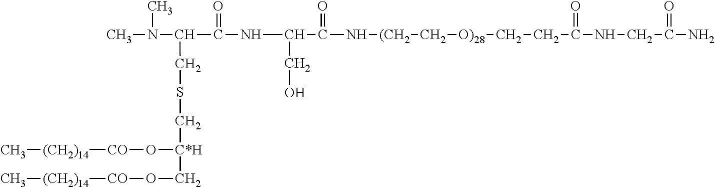 | A113 |
| 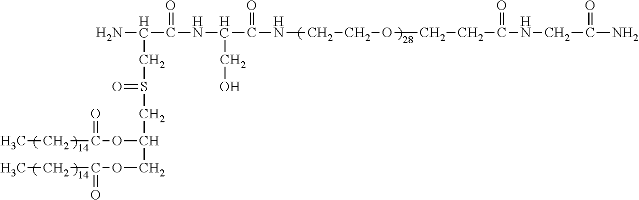 | A114 |
| 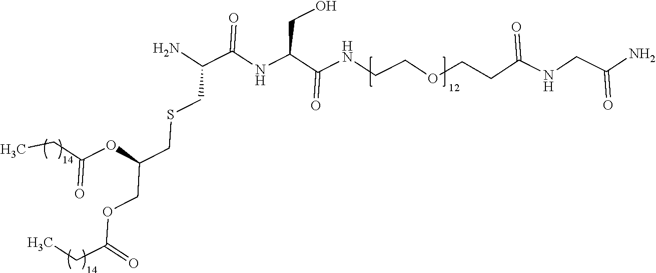 | A107 |
| 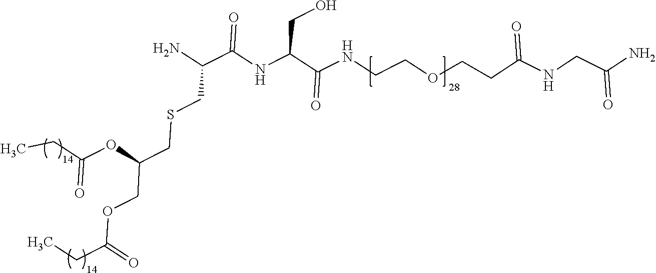 | A108 |
| 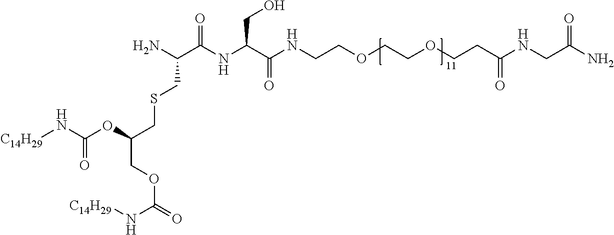 | A115 |
| 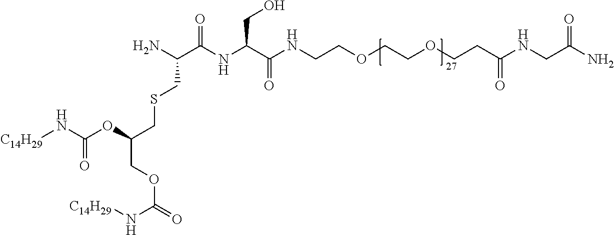 | A116 |
| 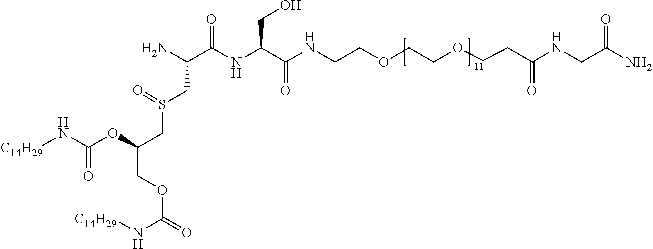 | A117 |
| 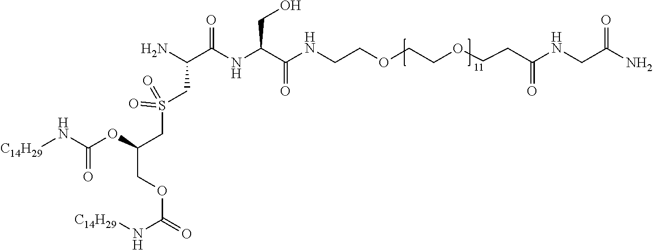 | A118 |
| 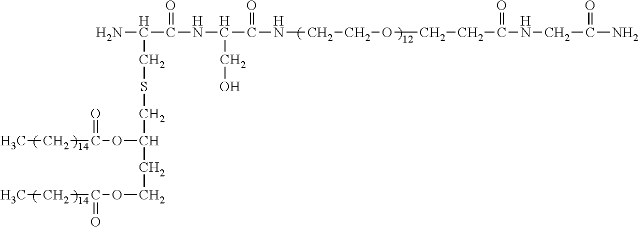 | A201 |
| 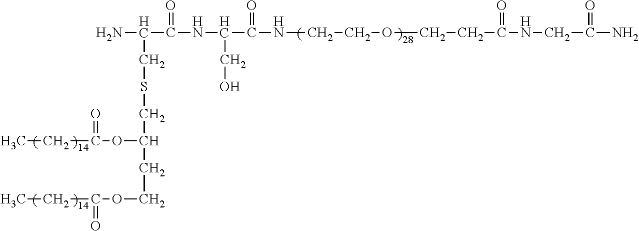 | A202 |
| 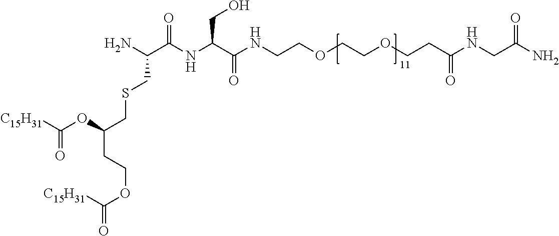 | A203 |
| 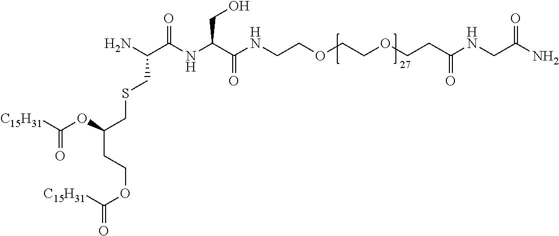 | A204 |
| 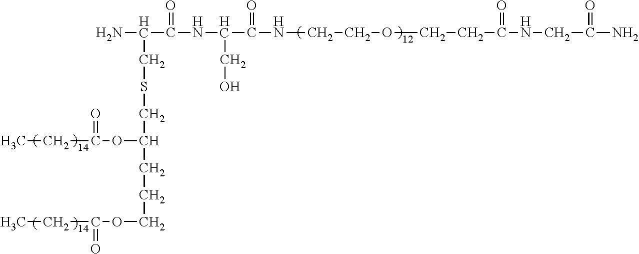 | A205 |
| 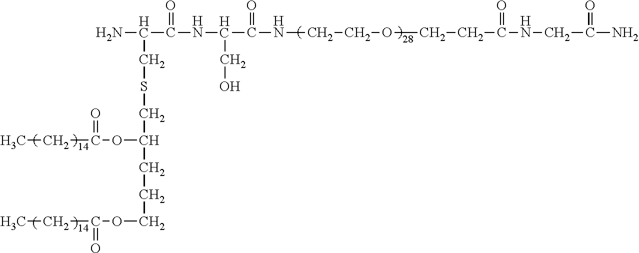 | A206 |
| 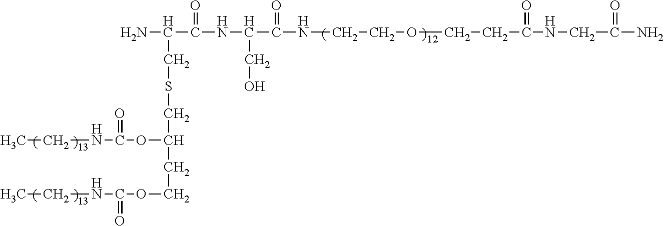 | A207 |
| 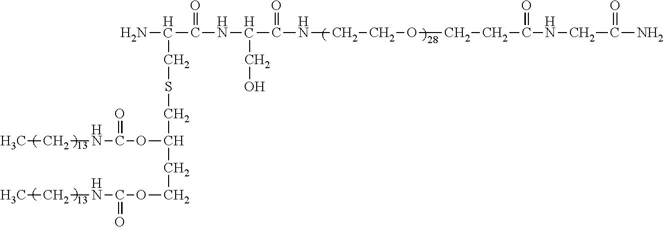 | A208 |
| 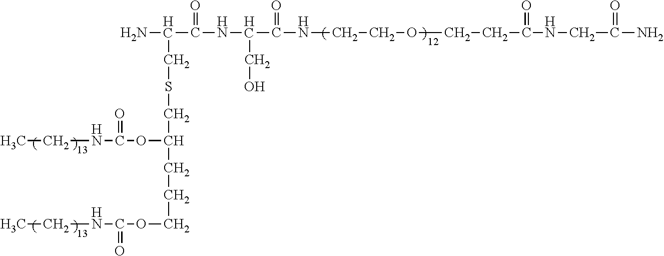 | A209 |
| 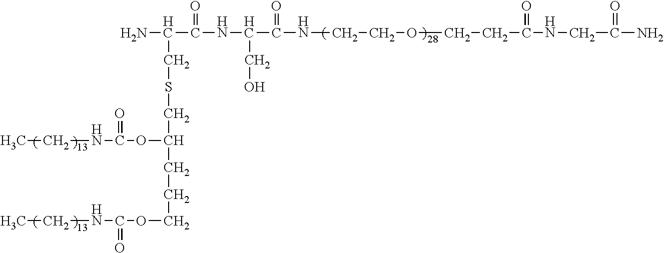 | A210 |
| 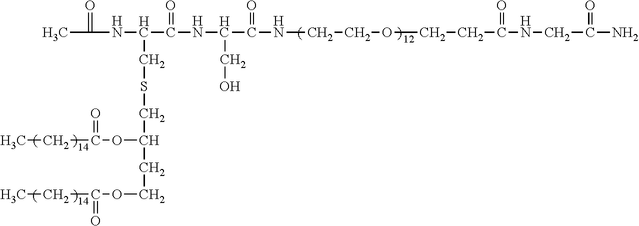 | A211 |
| 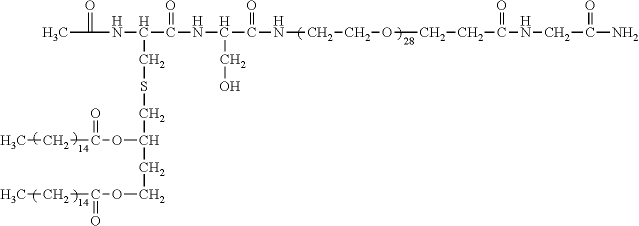 | A212 |
| 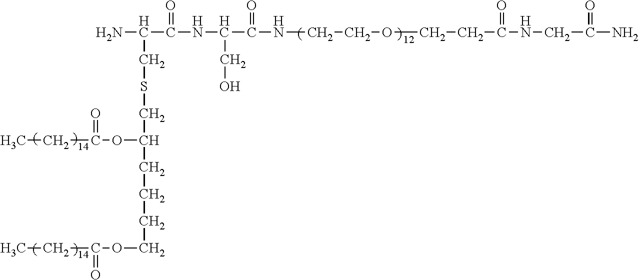 | A213 |
| 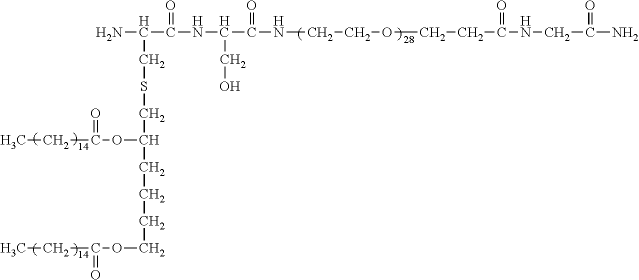 | A214 |
| 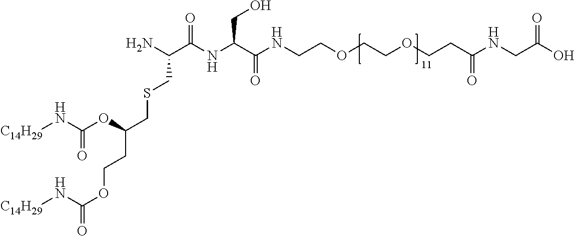 | A215 |
| 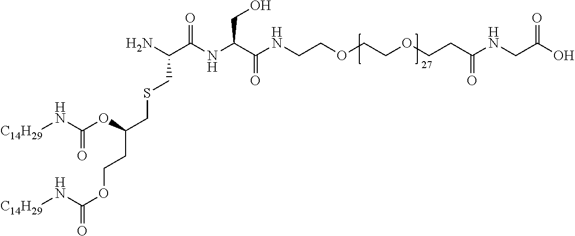 | A216 |
| 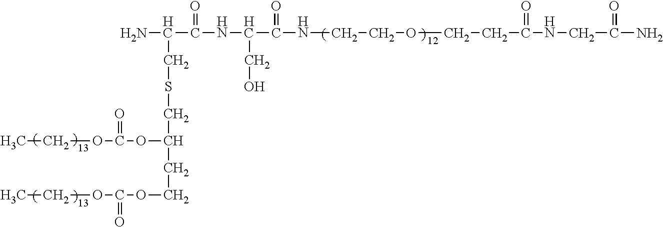 | A217 |
| 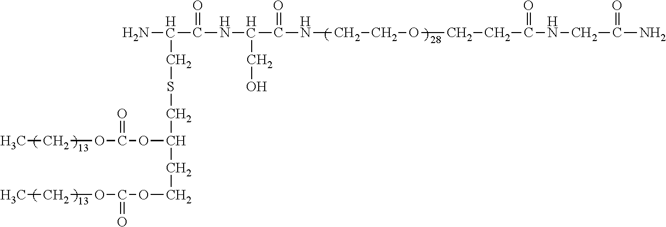 | A218 |
| 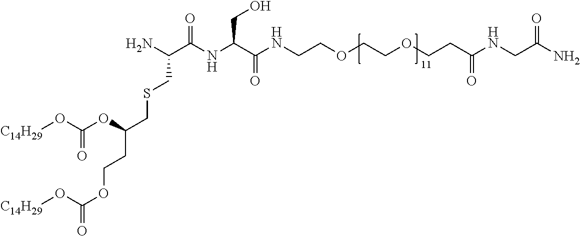 | A219 |
| 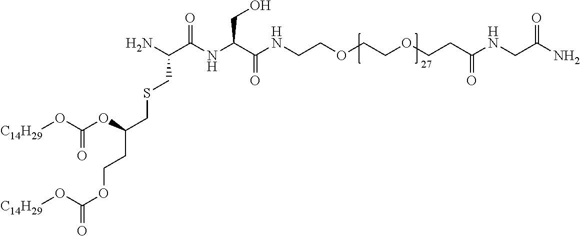 | A220 |
| 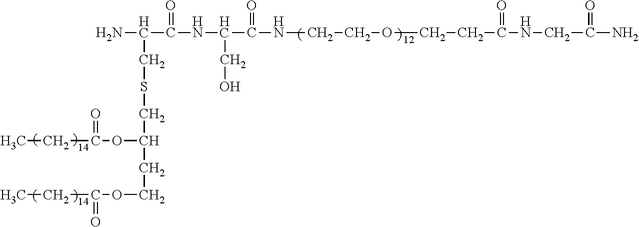 | A221 |
| 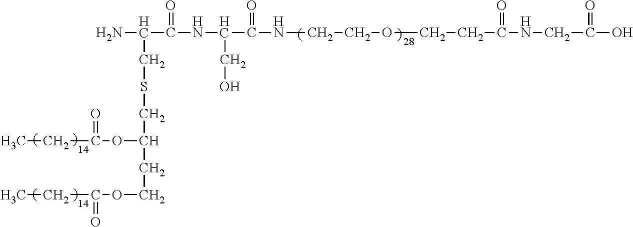 | A222 |
| 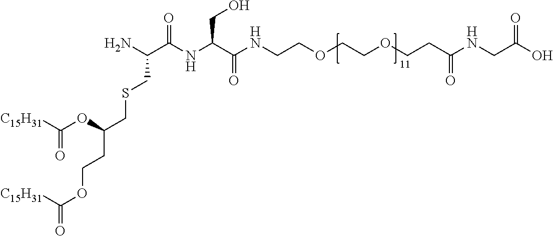 | A223 |
| 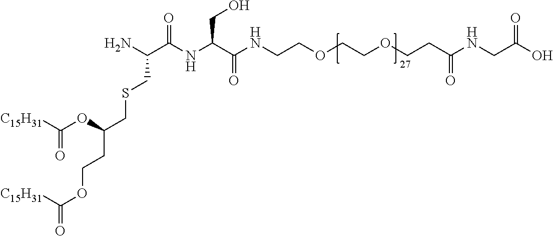 | A224 |
| 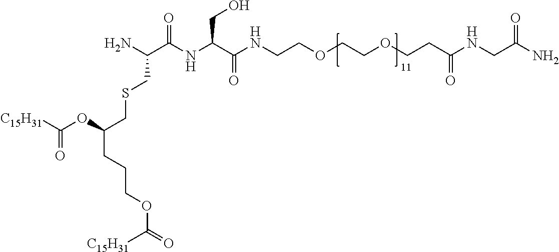 | A225 |
| 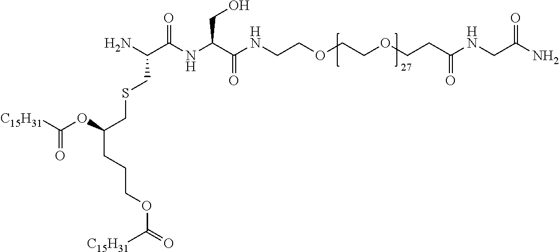 | A226 |
| 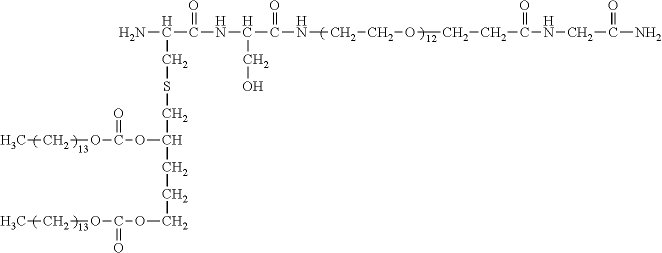 | A227 |
| 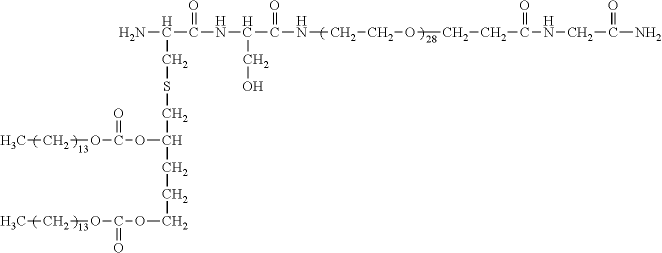 | A228 |
| 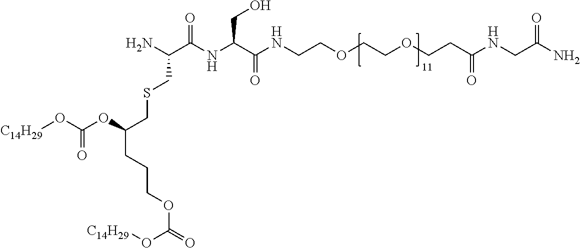 | A229 |
| 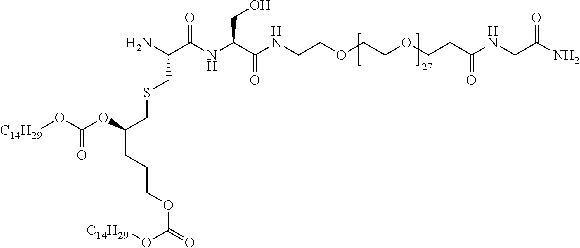 | A230 |
| 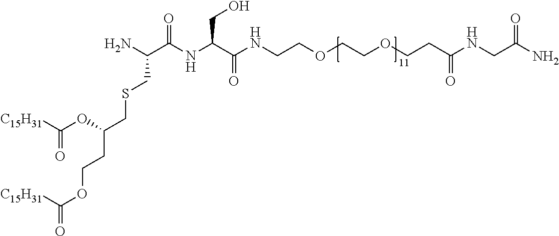 | A231 |
| 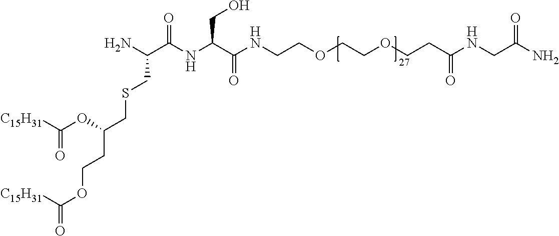 | A232 |

Compounds of the invention may be prepared by techniques known in the art. For example, compounds of the invention including any one of formulas (I)-(XIX) comprising an A1 moiety may be prepared by techniques described in WO2019/119067, the entire contents of which are hereby incorporated by reference.

Compounds of the invention including any one of formulas (I)-(XIX) comprising an A2 moiety may be provided by coupling a compound of the formula A2-I:

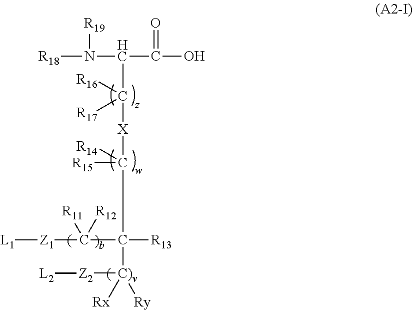

wherein L₁, L₂, Z₁, Z₂, v, b, w, z, Rₓ, R_{y}, R₁₁, R₁₂, R₁₃, R₁₄, R₁₅, R₁₆, R₁₇, R₁₈ and X have the meanings as defined for any compound of the invention defined herein and R₁₉ is an amino protecting group with a compound of formula (YB-1):

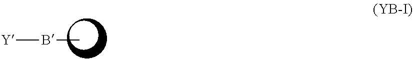

wherein

Y′ is

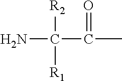

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

B′ is a Polyethylene Glycol (PEG); and

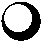

is a solid support resin.

In some embodiments, B′ comprises a substituted PEG of Formula B-I. In these embodiments, the following sequence of solid phase reactions may be employed:
a) Optionally coupling 1 to 10 alpha amino acids or compounds derived from a natural alpha amino acid, that constitutes L, to a solid phase resin using Fmoc chemistry
b) Coupling PG-NH—(CH₂)ₚ—O—(CH₂CH₂O)ₙ—(CH₂)ₘ—COOH to a solid phase resin or substituted resin if L is present, wherein PG represents an amino protecting group compatible with Fmoc chemistry;
c) Removing PG;
d) Coupling PG-NH—CR₁₃R₁₄—COOH, wherein PG′ represents an amino protecting group compatible with Fmoc chemistry;
e) Removing PG′;
f) Coupling an acid of the formula (A-I);
g) Optionally removing R₁₉ and optionally acylating and/or alkylating to introduce R₁₈ and/or R₁₉; and
h) Removing the compound from the solid phase support

In some embodiments, B′ comprises a substituted PEG according to formula (B-II) and the following sequence of solid phase reactions may be employed:
a) Optionally coupling 1 to 10 alpha amino acids or compounds derived from a natural alpha amino acid, that constitute L, to a solid phase resin using Fmoc chemistry
b) Coupling PG-NH—(CH₂)ₜ—O—(CH₂CH₂O)ₖ—(CH₂)ₕ—COOH to a solid phase resin or substituted resin if L is present, wherein PG represents an amino protecting group compatible with Fmoc chemistry;
c) Removing PG;
d) Coupling PG′-NH—(CH₂)ₚ—O—(CH₂CH₂O)ₙ—(CH₂)ₘ—COOH, wherein PG′ represents an amino protecting group compatible with Fmoc chemistry;
e) Removing PG′;
f) Coupling PG″-NH—CR₁₃R₁₄—COOH, wherein PG″ represents an amino protecting group compatible with Fmoc chemistry;
g) Removing PG″;
h) Coupling an acid of the formula (A-1);
i) Optionally removing R₁₉ and optionally acylating and/or alkylating to incorporate R₁₈ and/or R₁₉; and
j) Removing the compound from the solid phase resin.

It will be appreciated that the exact sequence of events can be varied from that outlined, and additional steps added where necessary and synthetically expedient, for example oxidation of the cysteine sulfur to the sulfoxide or sulfone.

In some embodiments, the compound of formula A2-1 is provided in the form of a compound of formula A2-II:

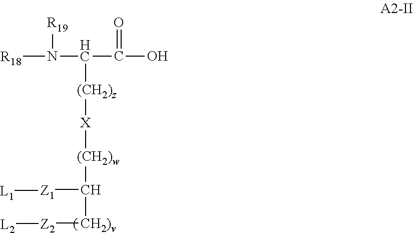

wherein L₁, L₂, X, v, w and R₁₈ are as defined for the compound of formula A-1 above, Z₁ and Z₂ are independently selected from —NHC(O)—, —C(O)NH—, —OC(O)—, —C(O)O—, —NHC(O)O— and —OC(O)O—.

The compound of formula A2-II may be prepared by the synthesis shown in Scheme 1.

Scheme 1 describes the synthesis of embodiments of the compound of formula A2-II,

wherein

X is S,

L₁-Z₁ are —OC(O)E-(CH₂)_{g}—CH₃, wherein E is —O— or —NH— and g is 10, 11, 12, 13, 14, 15, 16, 17 or 18;

L₂-Z₂ are —OC(O)E-(CH₂)_{g}—CH₃, wherein E is —O— or —NH— and g is 10, 11, 12, 13, 14, 15, 16, 17 or 18; and

R₁₉ is PG3, which is an amino protecting group.

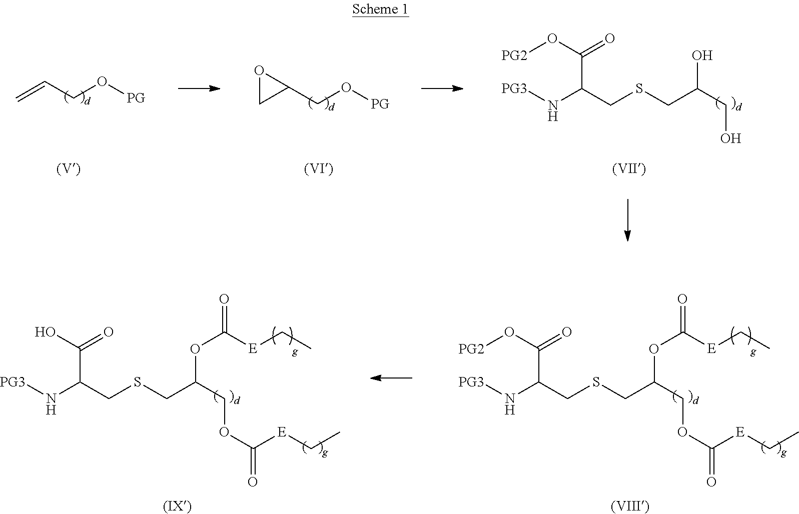

Reaction of protected alkene alcohols of the formula (V′), where PG is a suitable protecting group, for example a silyl group such as TBDMS, forms an epoxide of the formula (VI′). It will be appreciated that the epoxide formation maybe carried out to give the product racemically or to give enantioenriched material. If a racemic or scalemic mixture of enantiomers is produced preparative chiral chromatography is employed to separate the enantiomers if required.

Epoxides of the formula (VI′) are reacted with suitably protected cystine analogues, for example tert-butyl N-(((9H-fluoren-9-yl)methoxy)carbonyl)-S—(((R)-2-((((9H-fluoren-9-yl)methoxy)carbonyl)amino)-3-(tert-butoxy)-3-oxopropyl)thio)-D-cysteinate, where PG2 is a tert-butyl ester and PG3 is Fmoc, under reducing conditions to give alcohols of the formula (VII′). It will be appreciated that alcohols of the formula (VII′) can be comprised of more than one stereoisomer and where stereoisomers are present these can be separated by chiral preparative chromatography as required.

Alcohols of the formula (VII′) can be acylated to give carbonyl containing adducts of the formula (VIII′) using suitable reagents. Where esters are required, acid chlorides can be reacted in the presence of suitable bases and solvents; where carbamates are required isocyanates can be reacted in the presence of suitable bases and solvents and where carbonates are required chloroformates can be reacted in the presence of suitable bases and solvents. Carbonyl containing adducts of the formula (VIII′) can then be deprotected to reveal carboxylic acids of the formula (IX′) using suitable reagents, for example where PG2 is tert-butyl, trifluoroacetic acid can be used to preferentially remove the tert-butyl group.

Acids of the formula (IX′) can then be used as reagents in solid phase synthesis to add groups of formula Y and B.

Compounds of the invention including any one of formulas (I)-(XIX) comprising an A2 moiety wherein z is 1, w is 1 and b is 0, may be provided by preparing a resin bound peptide of the following formula:

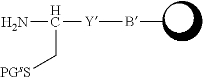

wherein

Y′ is

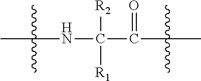

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;

R₈ is selected from the group consisting of H and a straight or branched C₁-C₆ alkyl;

B′ is a Polyethylene Glycol (PEG);

PG⁵ is H or a sulphur protecting group, such as tert-butyl; and

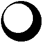

is a solid support resin.

Following optional sulphur deprotection, this resin bound peptide may be reacted with a 1,2-epoxy-alkanol of the following formula:

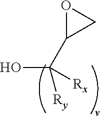

wherein Rₓ, R_{y} and v have the meanings given for moiety A2, for example as defined in formula (I)

to provide an alkylated thiol of formula S-1:

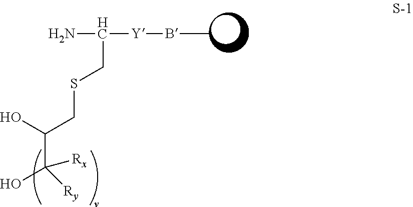

wherein Y′ and B′ have the meaning given above, and v has the meaning given for moiety A2, or a sulfone or sulfoxide thereof.

The diol moieties of resin bound compound S-1 may be further reacted to provide a compound of the invention, for example, by diol functionalisation with palmitic groups or lauryl carbamate groups, etc.

### EXAMPLES

The following examples 1-6 describe various properties of the compounds of the invention. The compounds tested in these examples were prepared as described in WO2019/119067 or in synthesis example 5.

### Example 1: Effect of Combination Therapy on Tumour Growth, Survival and Metastasis

Blocking inhibitory immune cell receptors also called “immune checkpoints” have revolutionized cancer treatment. Despite incredible response rates, the majority of patients do not respond or acquire resistance to immune checkpoint blockade (ICB). The underlying mechanisms are poorly understood and there is an unmet need to develop novel or improve existing immunotherapies. Here we set out to investigate the effect of combining a synthetic TLR2 agonist denoted compound A101 (also referred to as compound 1 in these Examples and Figures) with anti-PD1 immunotherapy in multiple models of cancer and cancer metastasis.

Material and Methods

Mice

C57BL/6 or Balb/c Wild-type (WT) mice were purchased from Walter and Eliza Hall Institute for Medical Research or bred in house and maintained at the QIMR Berghofer Medical Research Institute. Mice greater than 8 weeks of age were sex-matched to the appropriate models. The number of mice in each group treatment or strain of mice for each experiment is indicated in the figure legends. In all studies, no mice were excluded based on pre-established criteria and randomization was applied immediately prior to treatment in therapy experiments. Experiments were conducted as approved by the QIMR Berghofer Medical Research Institute Animal Ethics Committee.

Cell Culture

Mouse B16F10 (melanoma), MC38 (colon adenocarcinoma) and 4T1.2 (breast cancer) cells were grown in Dulbecco's Modified Eagle Medium (DMEM) supplemented with 10% Fetal Calf Serum (Bovogen), 1% Glutamine (Gibco), and 1% Penicillin/Streptomycin (Gibco). B16F10 and 4T1.2 cells were maintained at 37° C., 5% CO₂. MC38 cells were maintained at 37° C., 10% CO₂. All cell lines were routinely tested negative for Mycoplasma, but cell line authentication was not routinely performed.

Subcutaneous Tumor Models

For primary tumor growth experiments MC38 (1×10⁶) or 16F10 (1×10⁵) cells were s.c. injected into mice in a final volume of 100 μl (day 0). Treatment of mice commenced as indicated in figures or legends. Digital callipers were used to measure the perpendicular diameters of each individual tumor. The tumor size was calculated and is presented as mean±SEM. When tumours reached a size of 150 mm² mice were sacrificed.

4T1.2 Breast Cancer Metastasis Model

5×10⁴ 4T1.2 cells were injected into the 4^{th} mammary fat pad in a volume of 50 μl (day 0). On day 12 after injection, primary tumours were surgically removed under isoflurane anesthesia. Mice were injected treated as indicated in figure legends on day 15, 18 and 21 after tumour cell injection. All mice were sacrificed on day 26 after tumour cell injection for evaluation of metastatic burden. For this, macroscopic visible lung metastases were counted.

Immune Checkpoint Blockade Experiments

When transplanted tumours reached a size of 3-5 mm in diameter, immunotherapy was started. Therapeutic blockade of PD-1 was performed by i.p. injections of 250 μg rat anti-mouse PD-1 IgG2a (clone RMP1-14; BioXcell) or control-rat IgG2a mAb (clone 2A3; BioXcell) in 100 μl PBS. The amount of doses and the exact treatment schedules are depicted in the figures and/or legends.

Intratumoral Injection of Compound 1/Compound A101

When mice received the first injection of anti-PD1 or control IgG, mice were also treated with compound 1. For this 100 μl compound 1 solution in saline was injected into the tumour. Doses and treatment schedules are depicted in figures and/or legends.

Intravenous Injection of Compound 1/Compound A101

When mice received the first injection of anti-PD1 or control IgG, mice were also treated with compound 1. For this 10 μg compound 1 in 200 μl saline were injected into the lateral tail vein. Treatment schedules are depicted in figures and/or legends.

Results

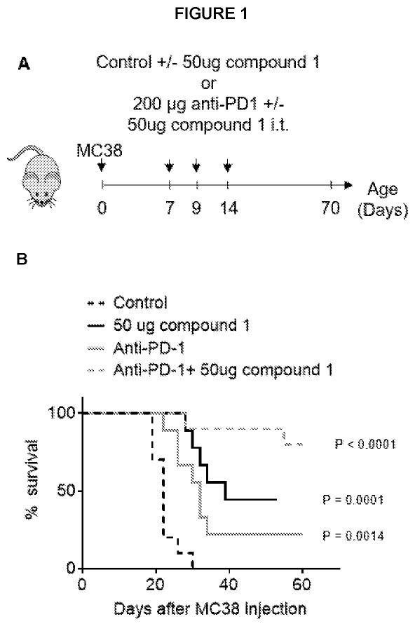

C57BL/6 WT mice were subcutaneously injected with MC38 cells and treated as indicated once tumours were palpable (FIG. 1A). Combining anti-PD1 immunotherapy with compound 1 significantly improved survival of MC38 tumour-bearing mice (FIG. 1B).

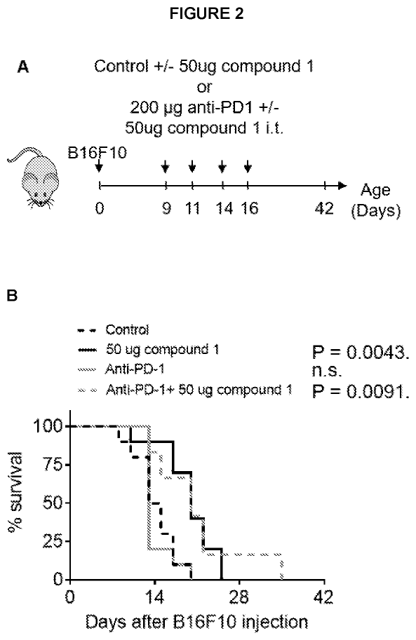

In the next experiment, the poorly immunogenic and immunotherapy resistant B16F10 melanoma cell line was injected into C57BL/6 WT mice. Again, when tumours were palpable, mice were randomized into four groups and treated as indicated (FIG. 2A). Surprisingly, there was improved survival in B16F10-bearing mice treated with compound 1 and anti-PD1 (FIG. 2B). Importantly, anti-PD1 monotherapy did not affect survival.

Taken together, it was shown that compound 1 effectively combines with anti-PD1 immunotherapy, when locally injected into the tumour microenvironment, in both highly and poorly immunogenic pre-clinical models of cancer.

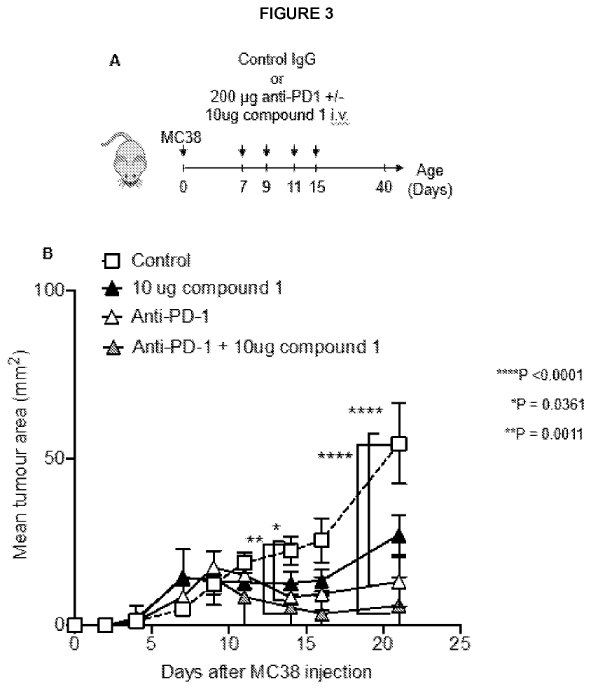

Whilst local injection is possible, it presents challenges in the day-to-day clinical routine. Moreover, many cancer entities are not accessible for intratumoral injections. Thus, the therapeutic efficacy of systemically administered compound 1 in combination with anti-PD1 immunotherapy was explored. For, this C57BL/6 WT mice were s.c. injected with MC38 cells. Once tumours were palpable mice were injected intravenously (i.v.) with 10 μg Compound 1 or control and intraperitoneally (i.p.) with anti-PD1 or control IgG (FIG. 3A). Combination immunotherapy of Compound 1 together with anti-PD1 was superior compared to anti-PD1 monotherapy (FIG. 3B).

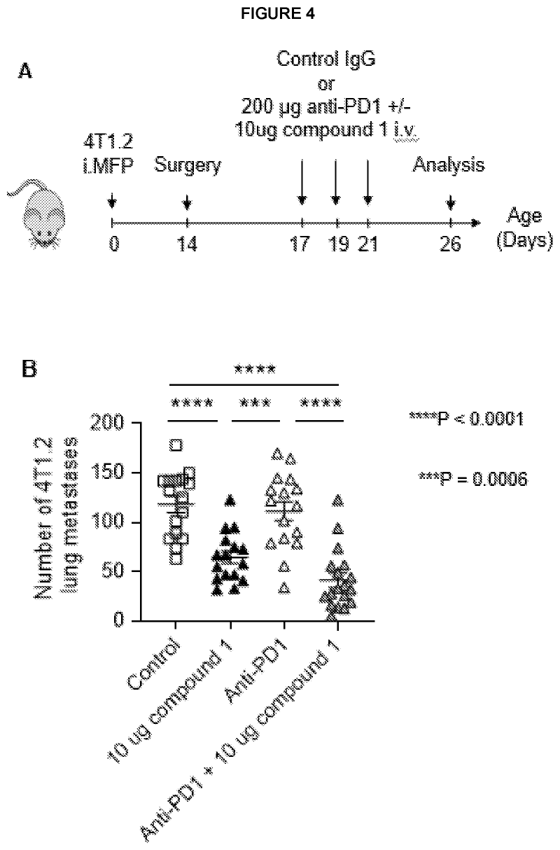

This data demonstrates that compound 1 improves the efficacy of anti-PD1 immunotherapy in the MC38 and B16F10 tumour models. As metastatic dissemination of tumour cells is the leading cause of death in cancer patients, the efficacy of combination immunotherapy in a spontaneous metastasis model was next evaluated. For this Balb/c WT mice were injected into the 4th mammary fat pad with the highly aggressive 4T1.2 breast cancer cell line. Similarly to the typical clinical approach, the subjects' primary tumours were surgically removed and mice treated with compound 1 alone or in combination with anti-PD1 immunotherapy (FIG. 4A). All mice were sacrificed 14 days after surgery and the number of lung metastases was evaluated. Primary 4T1.2 tumours as well as disseminated cancer cells are highly resistant to anti-PD1 immunotherapy. Systemic administration of 10 μg compound 1 resulted in lower numbers of lung metastases. Importantly, combination immunotherapy significantly reduced metastatic burden in mice (FIG. 4B), without affecting weight loss (data not shown).

In summary, we here show that compound 1, a highly specific TLR2 agonist, potentiates the efficacy of anti-PD1 immunotherapy providing a rationale for use of such combination therapies in the treatment of cancer of varying aetiology and pathogeneses. These results also demonstrate the utility of TLR2 agonist and checkpoint inhibitor combination therapies in cancers that were previously resistant to checkpoint inhibitor treatment.

### Example 2. Effect of Combination Therapy on Tumour Growth and Survival in the EMT6.5 Model

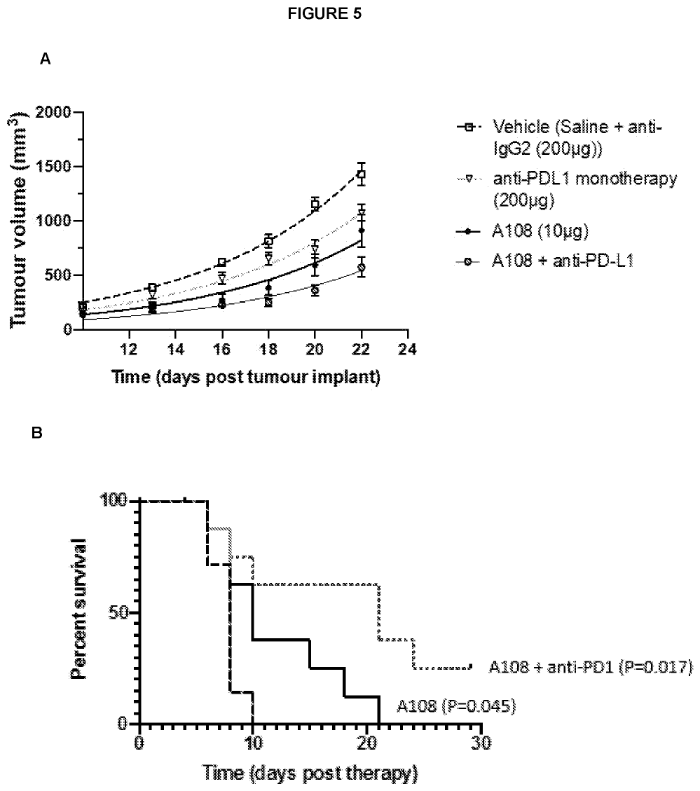

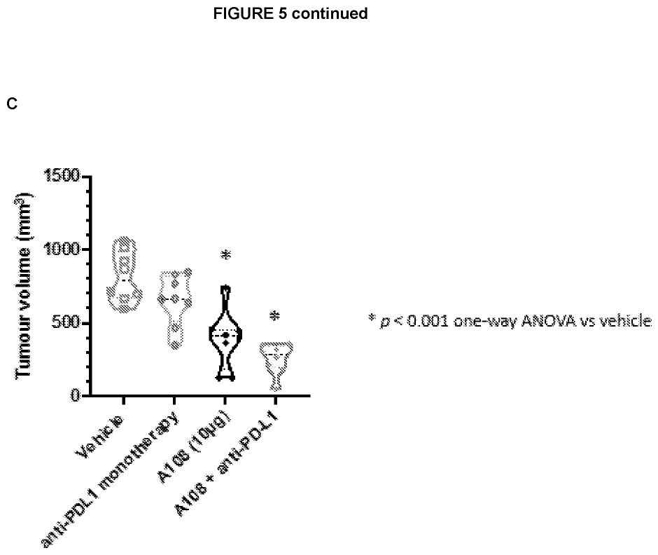

Balb/c mice (n=48) were implanted with 100,000 EMT6.5 cells into the 4^{th} inguinal mammary gland and established tumours monitored by calliper measurement. When tumours were ˜100 mm³ the mice were randomized to either one of six therapeutic arms (n=8 per arm). For the purpose of testing additional TLR2 agonists, compound A108 was administered intravenously to tumour bearing mice over three doses, three days apart. The checkpoint inhibitors were administered on the same day via the intra-peritoneal route. Therapeutic regimens containing compound A108 induced a mild but reversible loss of body weight (data not shown). The data shows that compound A108 in combination with anti-PDL-1 enhances the effect on tumour growth (FIG. 5A, C). Compound A108 in combination with anti-PL-1 also shows improved survival (FIG. 5B).

These data demonstrate that repeat dosing of systemically administered compound A108 is well tolerated in Balb/c mice bearing EMT6.5 tumours and that repeat dosing with compound A108 and anti-PDL-1 can statistically potentiate the effectiveness of different types of checkpoint inhibitors.

### Example 3. Effect of Combination Therapy on Tumour Growth and Survival of MC38-Bearing Mice

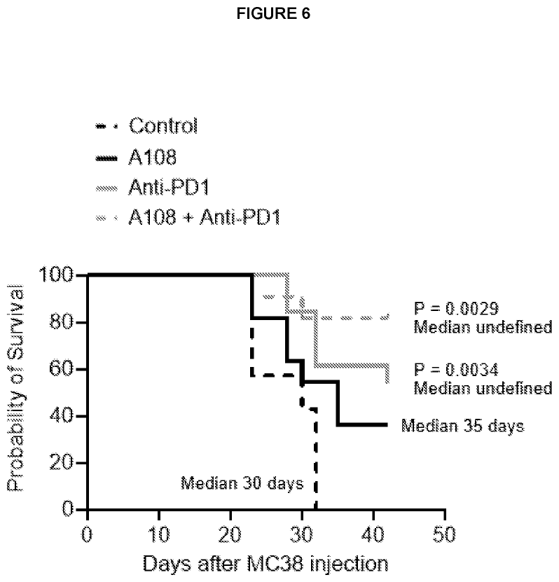

Compound A108 was also tested in the MC-38 model in the presence of checkpoint inhibitors. C57BL/6 WT mice (n=10-14) were injected subcutaneously with highly immunogenic MC38 colon carcinoma cells. Once tumours were palpable (˜3-5 mm in diameter), mice were randomized into four groups receiving three intra-tumoral injections of vehicle, anti-PD1 (200 μg, i.p), 25 μg compound A108 in 100 μl saline or compound A108 and anti-PD-1 in combination. This study demonstrates that the combination therapy improves the survival of mice (FIG. 6).

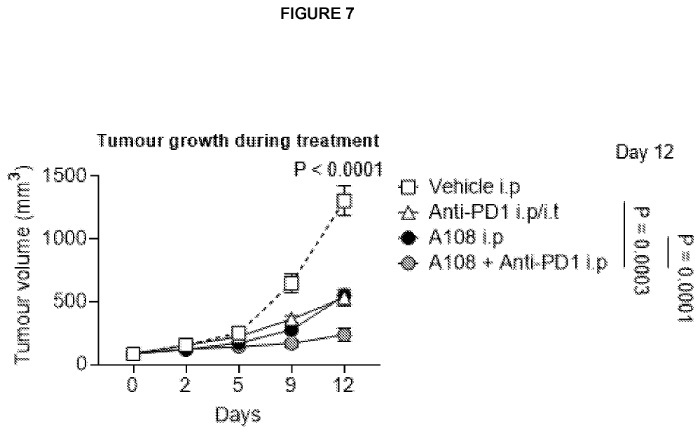

The above studies were explored further by examining the effect of the combination therapy on large tumours in the MC38 mouse model. These studies aimed to understand if repeated administration over a two-week period could slow tumour growth. Mice underwent repeated dosing every 2-days for 13 days via the i.p. (10 μg dose) route. Mice were culled when the humane endpoint was reached or one week following the final dose (up to day 25), whichever came first. Humane endpoints included body weight loss >20% relative to weight on the first day of treatment (or weight loss >15% for 3 consecutive days), individual mouse tumour volume >3000 mm³ or the mean tumour volume of a group >2000 mm³ (all mice in the group were culled). This study demonstrates that the combination therapy can slow tumour growth during the treatment phase via the i.p route (FIG. 7).

These data demonstrate that the combination of compound A108 and anti-PD1 immunotherapy can lead to impaired tumour growth and prolong survival of mice bearing MC38 colon carcinoma.

### Example 4. Compound A108 has Anti-Tumour Activity in the WEH1164 Model

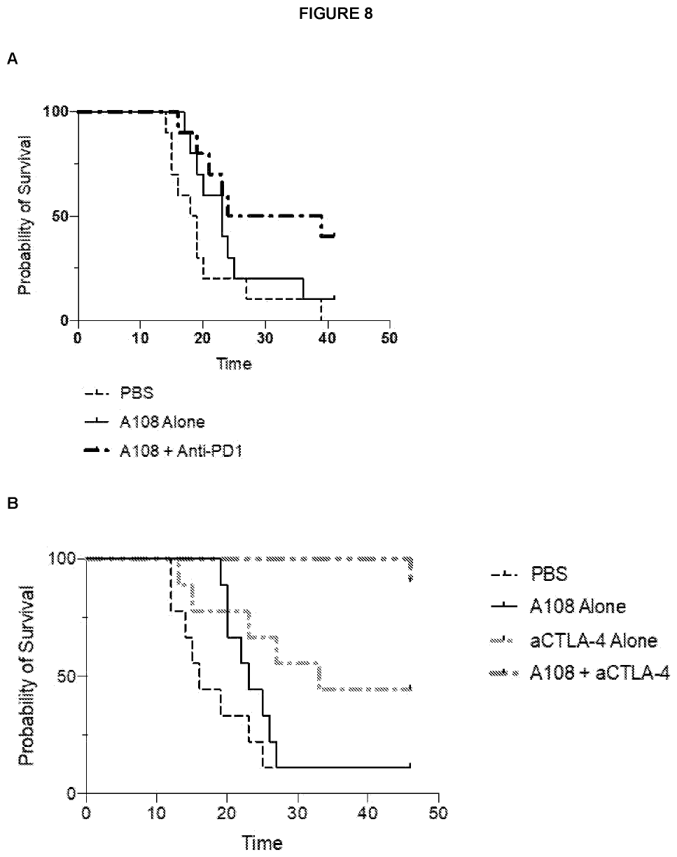

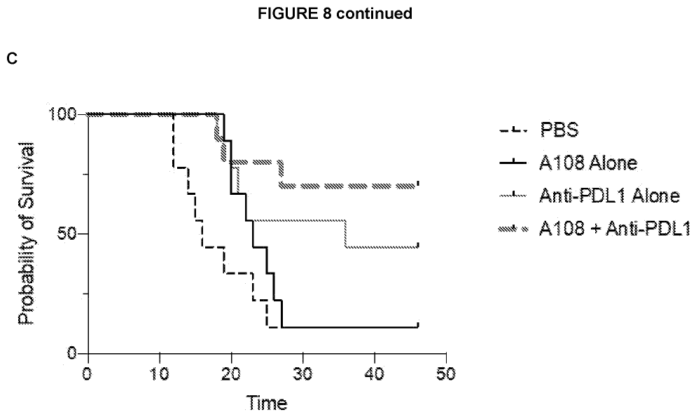

This study was conducted to determine whether a low dose of compound A108 (2.5 ug) in combination with anti-CTLA4, anti-PDL1 or anti-PD-1 could inhibit tumour growth. The data obtained from this experiment demonstrates that anti-PDL1 (FIG. 8A), anti-CTLA-4 (FIG. 8B) and anti-PD-1 (FIG. 8C) have a greater effect when administered in the presence of compound A108.

These data demonstrate that compound A108 increases the response rate to anti-CTLA4, anti-PDL1 and anti-PD-1 checkpoint therapy in the WEHI-164 sarcoma model of cancer.

### Example 5. Representative Synthesis, Characterisation and TLR2 Activity of Selected Compounds of the Invention: A107, A108, A115, A116, A117, A118, A203, A204, A215, A216, A220 and A224

Synthesis of Compounds A107 (x=11) and A108 (x=27)

Fmoc S-2,3-di(palmitoloxypropyl)-cysteine (S-Fmoc-Dpc-OH)_ was purchased from Bachem Inc.

Coupling of S-Fmoc-Dpc-OH to resin-bound peptide: Fmoc-Dpc-OH (100 mg, 0.24 mmol) is activated in DCM and DMF (1:1, v/v, 3 mL) with HOBt (36 mg, 0.24 mmol) and N,N′-diisopropylcarbodiimide (DIC; 37 uL, 0.24 mmol) at 0° C. for 5 min. The mixture is then added to a vessel containing the Boc-Cys-Ser(tBu)CH₂CH₂O—(PEG)₁₁-CH₂CH₂C(O)Gly resin or Boc-Cys-Ser(tBu)-CH₂CH₂O—(PEG)₂₇-CH₂CH₂C(O)Gly resin (0.25 mmol/g, 0.25 g=0.0625 mmole). After shaking for 2 h the solution is removed by filtration on a glass sinter funnel (porosity 3) and the resin washed with DCM and DMF (3×30 mL each). The reaction is monitored for completion using the trinitrobenezene sulfonic acid (TNBSA) test. If necessary a double coupling is performed.

Cleavage of peptide from the solid support: Reagent B (93% TFA, 5% water and 2% triisopropylsilane) for two hours. The peptide did not precipitate in chilled ether. Most of the TFA must be removed and then the residue is dissolved in 50% acetonitrile and purified immediately or freeze-dried.

Synthesis of Compounds A115 and A116.

Syntheses of compounds A115 (x=11) and A116 (x=27) were carried out as depicted in Scheme 2. (R)-glycidol is coupled to the thiol group of the cysteine residue attached to the peptide resin by alkylation; To 250 mg of Boc-Cys-Ser(tBu)CH₂CH₂O—(PEG)₁₁-CH₂CH₂C(O)Gly resin or Boc-Cys-Ser(tBu)CH₂CH₂O—PEG₂₇-CH₂CH₂C(O)Gly resin (0.25 mmole/g, 0.25 g=0.0625 mmole) saturated in DMF was added 250 μl of R-(+)-glycidol (MW=74.08, d=1.1, 250 μl=3.71 mmol, 60 fold excess over the free sulfhydryl group on the peptide resin) and 2511 of diisopropylethylamine (DIPEA, MW=129.2, d=0.74, 25 μl=0.14 mmol). The reaction mixture was held at 50° C. for 2 hrs in a water bath and then the solid support then thoroughly washed with DMF. To 250 mg of the peptide resin washed with toluene following glycidolation, were added 100 μl of ethylmethylsulfide (W=76.16, d=0.842, 100 μl=1.10 mmole) followed by 105 μl of tetradecyl isocyanate (MW=239, d=0.869, 105 μl=0.38 mmol, i.e. 3-fold excess over each of the hydroxyl groups present on the solid support) and finally 210 μl of dibutyltin dilaurate (MW=631.6, d=1.053, 210 μl=0.35 mmol). The reaction mixture was sparged with nitrogen gas for approximately 5 min and mixed (Intelli-Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage from the support was purified by HPLC.

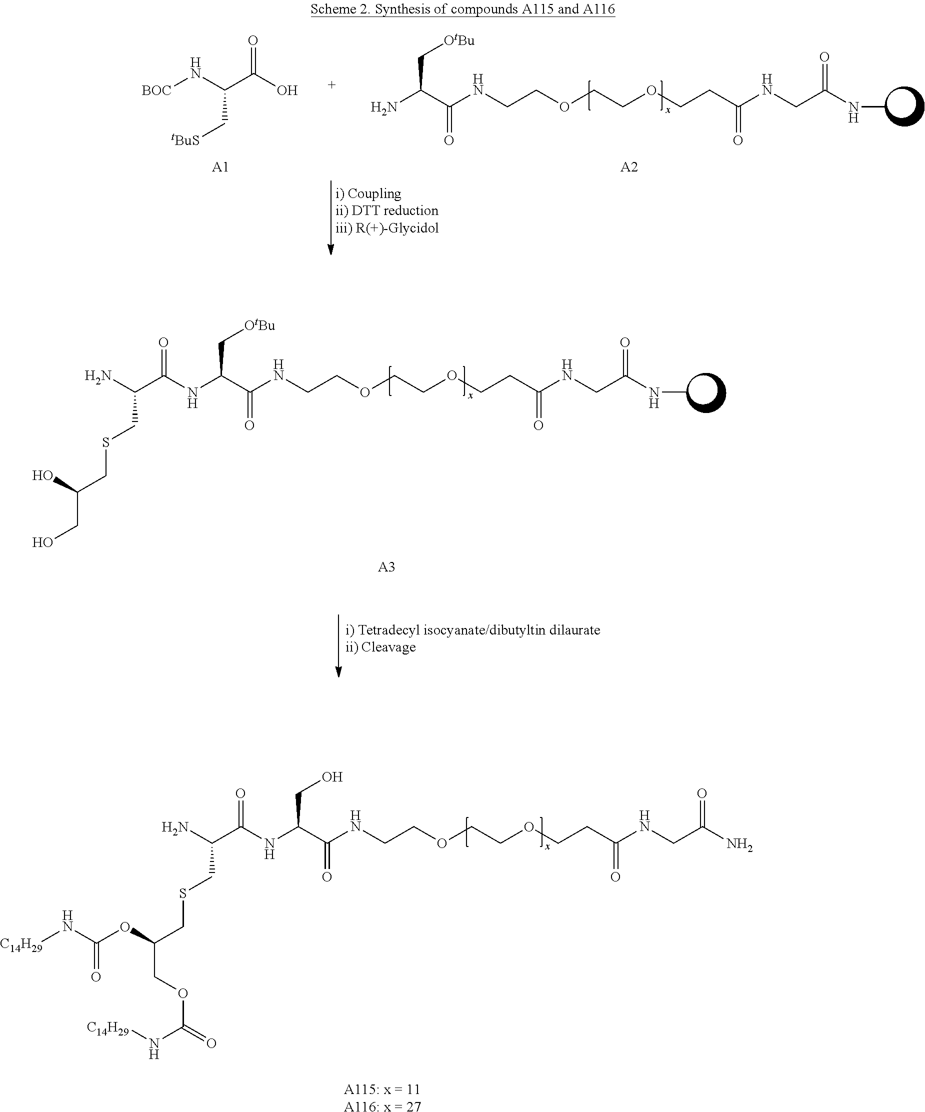

Synthesis of Compounds A117 and A118.

Compounds A117 (X═S(═O)) and A118 (X═S(═O)₂) were prepared following a similar synthetic routes as described above for compound A115, with the omission of ethylmethylsulfide scavenger, and optional omission of nitrogen sparging, from the carbamate formation step. Omitting the ethylmethylsulfide scavenger yielded a mixture of compounds A115, A117 and A118 which were separated and purified by HPLC.

Alternatively, sulfone or sulfoxide derivatives (eg A117 and A118) may be prepared by oxidation of the corresponding sulfide (eg A115) with an oxidant such as meta-chloroperoxybenzoic acid (MCPBA) or tert-butyl hydroperoxide (t-BuOOH) under appropriate conditions.

Synthesis of Compounds A203 and A204.

The synthesis of compounds A203 and A204 is depicted below in Scheme 3.

Fmoc-Gly was added as the first amino acid to the solid support, followed by coupling of Fmoc-NHCH₂CH₂O—(PEG)₁₁-CH₂CH₂COOH or Fmoc-NHCH₂CH₂O—(PEG)₂₇-CH₂CH₂COOH in 2-fold molar excess in presence of a two-fold excess of Hexafluorophosphate Benzotriazole Tetramethyl Uronium (HBTU), Hydroxybenzotriazole (HOBT) and 4-fold excess of diisopropylethylamine (DIPEA) in 2 ml of dimethylformamide (DMF) for 2 hrs. Fmoc-Ser(tBu)-OH is then coupled to provide intermediate A2, followed by the coupling of Boc-Cys(StBu) A1. The thiol-tert-butyl group on the cysteine residue was removed by incubating the peptide resin in 0.5M of dithiothreitol for 1 hr in DMF at RT. To 250 mg of Boc-Cys-Ser(tBu) —NHCH₂CH₂O—(PEG)₁₁-CH₂CH₂C(O)Gly resin or Boc-Cys-Ser(tBu) CH₂CH₂O—(PEG)₂₇-CH₂CH₂C(O)-Gly resin (0.25 mmole/g, 0.25 g=0.0625 mmole) saturated in DMF was added 250 μl of R-(+)-1,2-epoxy-butan-4-ol [(R)-2-(oxiran-2-yl)ethan-1-ol] (M_{W}=88.11, d=1.1, 250 μl=3.125 mmol equivalent to a 50 fold excess over the free sulfhydryl group present on the peptide resin) and 25 μl of diisopropylethylamine (DIPEA, M_{W}=129.2, d=0.74, 25 μl=0.14 mmol). The reaction mixture was left in a water bath at 50° C. for 2 hrs and then thoroughly washed with DMF to provide intermediate A3.

Palmitic acid (320 mg, 1.25 mmol), DIPCDI (225 uL, 1.5 mmol) and 4-dimethylaminopyridine (DMAP; 15.25 mg, 0.125 mmol) were dissolved in 2 mL of dichloromethane (DCM) then added to the resin-bound BOC-Dhc-peptide resin A3 (0.0625 mmol, 0.25 g) and shaken for 16 h at room temperature. The supernatant was removed by filtration and the solid support thoroughly washed with DCM and dimethylformamide (DMF) to remove any residue of urea before being subjected to the cleavage process as described below.

The solid support bearing the assembled lipopeptide was exposed to reagent B (93% TFA, 5% water and 2% triisopropylsilane) for 2 hours. To isolate the product, most of the TFA was removed and the residue is then dissolved in 50% acetonitrile and purified immediately using the purification protocol described below or the material was freeze-dried and stored for later purification.

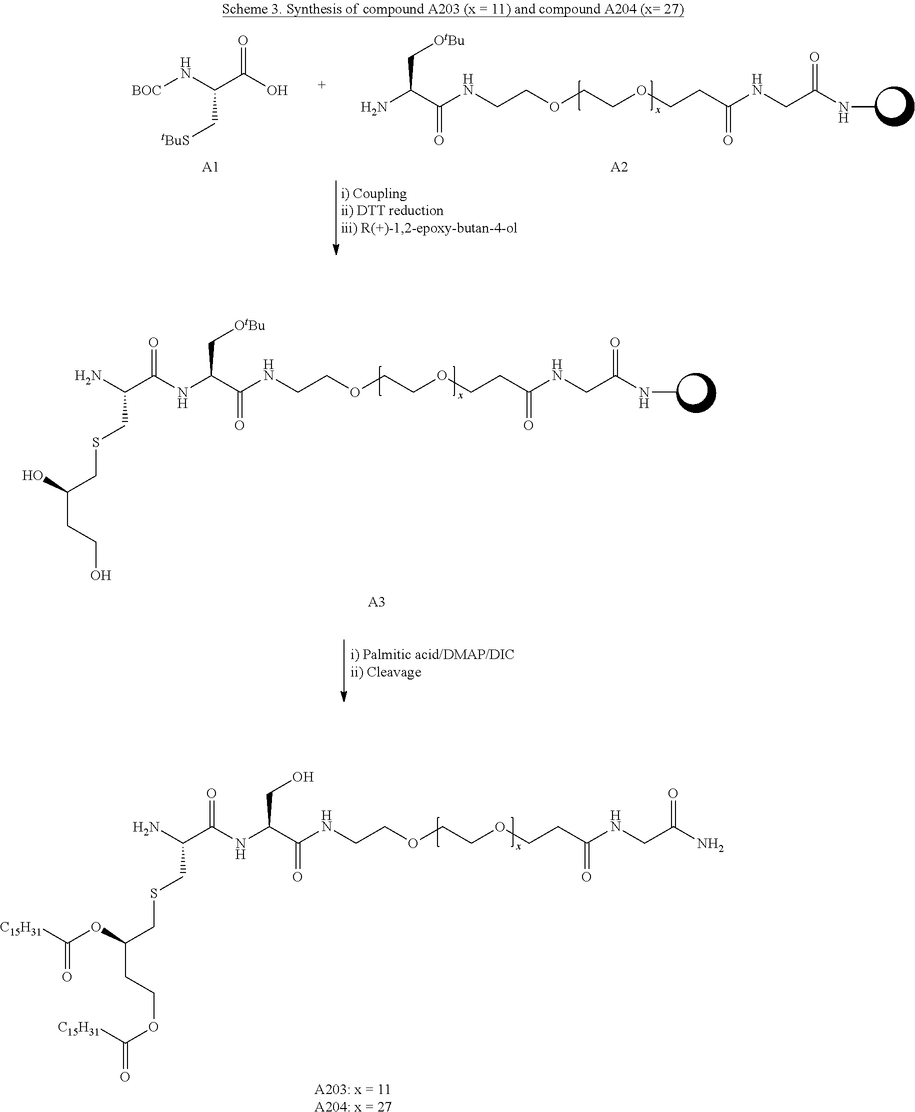

Synthesis of Compound A215 and Compound A216.

The synthesis of compounds A215 and A216 was carried out as depicted in Scheme 4. Intermediate A3 was prepared as described for compounds 3 and 4 above.

Then, to 250 mg of the peptide resin washed with toluene following glycidolation, were added 100 μl of ethylmethylsulfide (M_{W}=76.16, d=0.842, 100 μl=1.10 mmol) followed by 105 μl of tetradecyl isocyanate (M_{W}=239, d=0.869, 105 μl=0.38 mmol, i.e. 3-fold excess over each of the hydroxyl groups present on the solid support) and finally 210 μl of dibutyltin dilaurate (M_{W}=631.6, d=1.053, 210 μl=0.35 mmol). The reaction mixture was sparged with nitrogen gas for approximately 5 min and mixed (Intelli-Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage (as above) from the support was purified by HPLC.

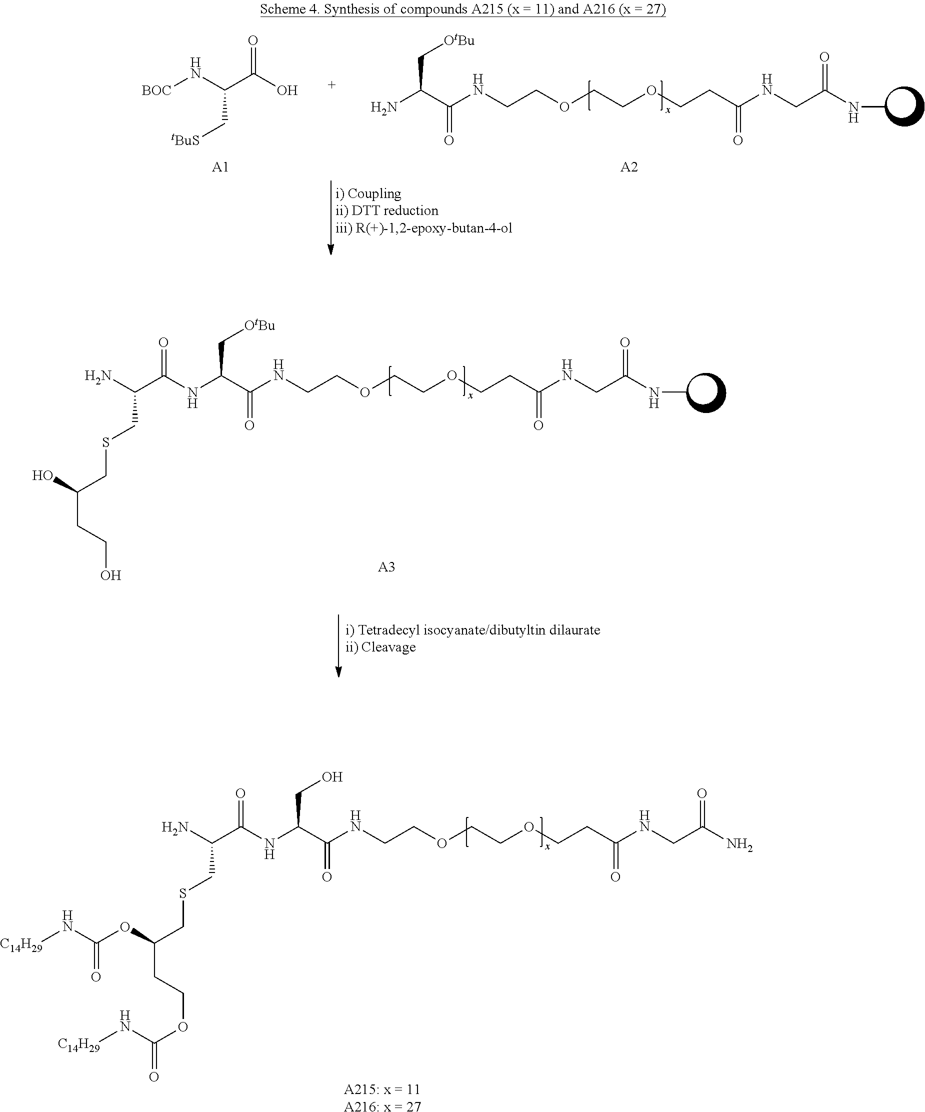

Synthesis of A220. Compound A220 was synthesized by standard Fmoc Solid Phase Peptide Synthesis, starting with Fmoc-RINK MBHA PS Resin. Removal of the Fmoc group after each coupling was achieved using 20% piperidine in DMF. Couplings of Fmoc-Gly-OH (2-fold excess), Fmoc-NH-PEG₂₃-CH₂CH₂COOH (1.4-fold excess), Fmoc-Ser(tBu)-OH (2-fold excess), and N-(Boc)-S—((R)-2,3-dihydroxybutyl)-L-cysteine (1.5-fold excess) were performed in DMF using equivalent excess of ethyl cyano(hydroxyimino)acetate (Oxyma Pure) and diisopropylcarbodiimide (DIC) as coupling agents. Myristyl Chloroformate coupling was performed using Myristyl Chloroformate (12 eq. vs. moles resin), DIEA (24 eq. vs. moles resin) in dry DCM for 18 hours at room temperature. This coupling was repeated three times (“recoupling”). The first recoupling was done using Myristyl Chloroformate (12 eq. vs. moles resin), NMM (24 eq. vs. moles resin) in dry DCM/THF (85/15) for 18 hours at room temperature. The second recoupling was done using Myristyl Chloroformate (6 eq. vs. moles resin), NMM (12 eq. vs. moles resin) in dry DCM/THF (85/15) for 41 hours at room temperature. Finally the third recoupling was performed using Myristyl Chloroformate (6 eq. vs. moles resin), NMM (12 eq. vs. moles resin) in dry DCM/THF/Toluene (85/15/5) for 21.5 hours at room temperature.

Cleavage of the peptide from the resin, removal of N-terminal Boc group, and serine side-chain deprotection were achieved by exposure of the resin to a solution of 93% trifluoroacetic acid (TFA), 5% H₂O, 3% triisopropylsilane (TIPS) for 1.5 hours. Following the cleavage reaction, the mixture was evaporated and the resulting residue was re-dissolved in 30% acetonitrile/water and lyophilized.

Synthesis of A224. Compound A224 was synthesized by standard Fmoc Solid Phase Peptide Synthesis, starting with Chlorotrityl Chloride Resin with an initial substitution of 1.6 meq/g. The first amino acid, Fmoc-Gly-OH, was loaded on the resin first, using a 0.5-fold molar excess of Fmoc-Gly-OH and DIEA (1.5-fold excess), followed by capping with DMF/MeOH/DIEA (80/10/10), and Fmoc deprotection, to obtain the dry loaded H-Gly-CT Resin with a final substitution of 0.67 meq/g. Removal of the Fmoc group after each coupling was achieved using 20% Piperidine in DMF. Coupling of Fmoc-NH-PEG₂₃-CH₂CH₂COOH (1.4 eq.) was performed using (7-Azabenzotriazol-1-yloxy)trispyrrolidinophosphonium hexafluorophosphate (PyAOp; 1.4 eq.), diisopropylethylamine (DIEA; 3.2 eq.) in DMF, whereas couplings of Fmoc-Ser(tBu)-OH (2 eq), and N-(Boc)-S—((R)-2,4-dihydroxybutyl)-L-cysteine (1.5 eq.) were performed in DMF using equivalent excess of Oxyma Pure and DIC as coupling agents. Palmitic Acid coupling was performed using palmitic acid (20 eq. vs. moles resin), DIC (20 eq.), DMAP (2 eq.) in DCM/THF (85/15) (v/v) for 24 hours at room temperature.

Cleavage of the peptide from the resin, removal of N-terminal Boc group, and serine side-chain deprotection were achieved by exposure of the resin to a solution of 93% TFA, 5% H2O, 3% TIPS for 1.5 hours. Following the cleavage reaction, the mixture was evaporated and the resulting residue was re-dissolved in 30% Acetonitrile/Water and lyophilized.

Purification and Characterisation

Purification and characterisation: Following cleavage from the solid support, each of the analogs were purified by reversed-phase HPLC according to either protocol A or B described below.

Protocol A: Reversed phase HPLC was conducted using an Agilent Zorbax 300SB-C3, Sum column (9.4 mm×250 mm; Agilent Technology, Australia) installed in an Agilent HPLC 1260 Infinity system (Agilent Technologies, Santa Clara, Calif., USA) with the chromatogram developed using Buffer A (0.1% trifluoroacetic acid in water) and buffer B (0.1% trifluoroacetic acid in acetonitrile).

Protocol B: Reverse phase chromatography was conducted using a Novasep Axial Compression Column (5-cm diameter) loaded with cyano media (Daisogel SP-120-CN-P), with a gradient of Acetonitrile in [0.1% TFA/Water]. Following intermediate lyophilization, ion-exchange was performed on Dowex ion-exchange resin in order to obtain the peptide as the acetate salt.

Identification and purity determination of the target materials were carried out using an in-line HPLC-MS system using the following conditions:

Conditions A: HPLC column: Agilent Zorbax 300-SB C3 (150×0.5 mm; 5 μm) with the following gradient conditions: 0-5 min, 20% B: 5-32 min, 20% B-100% B: 32-40 min, 100% B-20% B. The flow rate was 20 μl/min. LC-MS: Agilent 1100 series capillary LC system in-line with an Agilent 1100 series LC/MSD ion-trap mass spectrometer. The mass spectrometer was operated with electrospray ionisation configured in the positive ion mode. Data analysis software from Agilent Technologies was used to de-convolute the charged ion series for identification of the peptide material and the material then characterised by LC-MS.

Conditions B: analytical reverse phase HPLC with a cyano column (Daiso Fine Chem, SP-120-3-CN-P, 150×4.6 mm, 3 μm, 120 Å). The peptide was also analyzed by ESI LC-MS in Positive Ion Mode, using a Finnigan LCQ Deca XPMax.

Compounds A107, A108, A115, A116, A203, A204, A215 and A216 following protocol A and conditions A, compounds A220 and A224 were prepared and purified as described above following protocol B and conditions B, and prepared and purified as described above, were each found to be greater than 95% pure.

Peptide Quantitation

Quantitation of compounds A107, A108, A115, A116, A203, A204, A215 and A216 was carried out by in vacuo hydrolysis at 110° C. of samples in sealed glass vials in the presence of 6N HCl containing 0.1% phenol. Derivatisation of amino acids was then carried out using Waters AccQTag reagents according to the manufacturer's instructions followed by analysis on a Waters Acquity UPLC System (Waters Millipore) using an AccQTag ultra column (2.1 mm×100 mm; Waters Millipore). Quantitation of other compounds may be achieved by a similar protocol.

Synthesis of Sulfone and Sulfoxide Analogues of Compounds A215 and A216

Sulfone and sulfoxide derivatives of compounds A215 and A216 may be accessed by a similar synthetic routes as described above, with the omission of ethylmethylsulfide scavenger, and optional omission of nitrogen sparging, from the carbamate formation step. This reaction may yield a mixture of thiol, sulfone and sulfoxide derivatives, which may be separated and purified by HPLC.

Alternatively, sulfone or sulfoxide derivatives may be prepared by oxidation of the corresponding sulfide with an oxidant such as meta-chloroperoxybenzoic acid (MCPBA) or tert-butyl hydroperoxide (t-BuOOH) under appropriate conditions.

Activation of Human TLR2

The potency of the compounds as activators of human and mouse TLR-2s is tested in an in vitro assay. The assay assesses NF-kB activation in the HEKBlue-mTLR-2 cell line. These cells have been stably transfected with mouse TLR-2 and express TLR-1 and TLR-6 endogenously at sufficient levels to allow for fully-functional TLR-1/2 and TLR-2/6 activation.

Toll-Like Receptor 2 (TLR2) stimulation is tested by assessing NF-kB activation in the HEKBlue-hTLR2 cell line. These cells have been stably transfected with human TLR2 and express TLR1 and TLR6 endogenously at a level sufficient to allow for fully-functional TLR1/2 and TLR2/6 activation. The activity of the test articles are tested on human TLR2 as potential agonists. The test articles are evaluated at seven concentrations and compared to control ligands. These steps are performed in triplicate.

NF-kB reporter gene assay protocol: This assay was carried out as described previously (Jackson et al. 2004; Lau et al. 2006; Sandor et al. 2003; Zeng et al 2010). HEK293T cells were cultured in 96-well plates at 4×10⁴ cells/well and transfected 24 h later with 100 ng of the NF-kB luciferase reporter gene [50 ng of TK-Renilla-luciferase expressing plasmid (Promega corporation, Madison, USA)] with or without 5 ng TLR2-expressing plasmid in the presence of 0.8 μl Fugene 6 (Roche Diagnostic). Compounds were added to the wells 24 h later at the concentrations indicated in the histograms. Cell lysates were prepared 5 h after stimulation using reporter lysis buffer (Promega Corporation, Madison, USA). Luciferase activities in the cell lysates were determined using a reagent kit (Promega Corporation, Madison, USA) and using a FLUOstar microplate reader (BMG Labtech, Ortenberg, Germany). The NF-kB-dependent firefly luciferase activity is normalised with NF-kB-independent renilla luciferase activity. The relative stimulation was calculated as the ratio of the stimulated to non-stimulated samples.

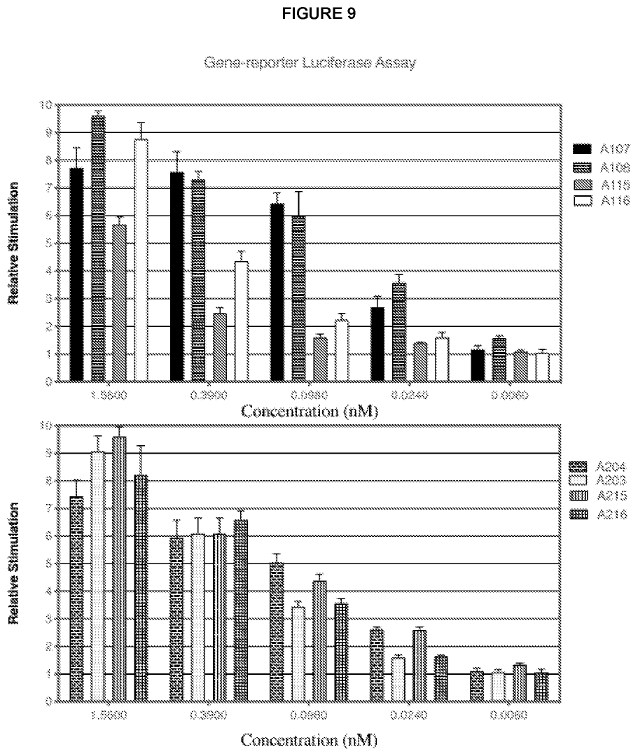

The results of this assay for compounds A107, A108, A115, A116, A203, A204, A215 and A216 are shown in FIG. 9. These data show that these compounds exhibit significant activity at TLR2.

1. A method of treating, preventing or minimising progression of cancer in a subject comprising administering a therapeutically effective amount of a TLR2 agonist and an immunostimulant to a subject, thereby treating, preventing or minimising progression of cancer in the subject.

2. A method of treating, preventing or minimising the progression of cancer in a subject who has received, or who is receiving, an immunostimulant comprising administering a therapeutically effective amount of a TLR2 agonist to the subject, thereby treating, preventing or minimising progression of cancer in the subject.

3. A method of treating, preventing or minimising progression of cancer in a subject comprising the steps of:
identifying a subject having cancer who has received, or who is receiving an immunostimulant for the treatment of cancer,
assessing whether the subject is responsive to the immunostimulant,
administering a therapeutically effective amount of a TLR2 agonist to the subject if the subject is not responsive to the immunostimulant,
thereby treating, preventing or minimising progression of cancer in the subject.

4. A method of treating, preventing or minimising progression of cancer in a subject comprising the steps of:
identifying a subject having cancer and being unresponsive to a treatment comprising an immunostimulant,
administering a therapeutically effective amount of a TLR2 agonist to the subject,
thereby treating, preventing or minimising progression of cancer in the subject.

5. A method of treating, preventing or minimising progression of cancer in a subject comprising the steps of:
identifying a subject having cancer; and
administering a therapeutically effective amount of a TLR2 agonist and an immunostimulant to the subject,
thereby treating, preventing or minimising progression of cancer in the subject.

6. A method of increasing survival of a subject having cancer comprising administering a therapeutically effective amount of a TLR2 agonist and an immunostimulant to a subject, thereby increasing survival of the subject having cancer.

7. A method of minimising, reducing or preventing growth of a tumour in a subject having cancer comprising administering a therapeutically effective amount of a TLR2 agonist and an immunostimulant to a subject, thereby minimising, reducing or preventing growth of a tumour in the subject having cancer.

8. A method of minimising, reducing or preventing metastasis in a subject having cancer comprising administering a therapeutically effective amount TLR2 agonist and an immunostimulant to a subject, thereby minimising, reducing or preventing metastasis in the subject having cancer.

9. The method of claim 8, wherein the method minimises, reduces or prevents metastasis to the lung.

10. A method of minimising, reducing or preventing cancer in a subject comprising:
identifying a subject having a tumour capable of metastasising; and
administering a therapeutically effective amount of a TLR2 agonist and an immunostimulant to a subject,
thereby minimising, reducing or preventing cancer in the subject.

11. Use of a TLR2 agonist an immunostimulant in the preparation of a medicament for treating, preventing or minimising progression of cancer in a subject.

12. Use of a TLR2 agonist and an immunostimulant for treating, preventing or minimising progression of cancer in a subject.

13. The method or use according to any one of claims 1 to 12, wherein the immunostimulant is selected from the group consisting of cellular immunotherapies, oncolytic viruses, cancer vaccines, T-cell engagers, bispecific T-cell engagers and checkpoint inhibitors.

14. The method or use according to claim 13, wherein the immunostimulant is a checkpoint inhibitor.

15. The method or use according to claim 14, wherein the checkpoint inhibitor is a PD-1, PD-L1 or a CTLA-4 checkpoint inhibitor, preferably in the form of an antibody.

16. The method or use according to claim 15, wherein the checkpoint inhibitor is a PD-1 antibody.

17. The method or use according to any one of claims 1 to 16, wherein the cancer is selected from the group consisting of breast cancer, colorectal cancer, adenocarcinomas, mesothelioma, bladder cancer, prostate cancer, germ cell cancer, hepatoma/cholongio carcinoma, neuroendocrine cancer, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, fibrosarcoma, cervical tumours, esophageal tumours, laryngeal tumours, liver tumours, lung tumours, vaginal tumours or Wilm's tumour. In a preferred embodiment, the cancer is melanoma or colon cancer.

18. The method or use according to claim 17, wherein the cancer is melanoma, breast cancer, fibrosarcoma or colon cancer.

19. The method or use according to any one of claims 1 to 18, wherein the TLR2 agonist is administered in a composition that further comprises a pharmaceutically acceptable carrier, diluent or excipient.

20. The method or use according to claim 19, wherein the composition consists of a TLR2 agonist, an immunostimulant and a pharmaceutically acceptable carrier, diluent or excipient.

21. The method or use according to any one of claims 1 to 20, wherein the TLR2 agonist comprises a lipid, a peptidoglycan, a lipoprotein or a lipopolysaccharide.

22. The method or use according to any one of claims 1 to 21, wherein the TLR2 agonist comprises Pam₂Cys-Ser-PEG.

23. The method or use according to any one of claims 1 to 22, wherein the checkpoint inhibitor is a PD-1 antibody and the TLR2 agonist comprises Pam₂Cys-Ser-PEG.

24. The method or use according to any one of claims 1 to 23, wherein the TLR2 agonist comprises palmitoyl, myristoyl, stearoyl, lauroyl, octanoyl, or decanoyl.

25. The method or use according to any one of claims 1 to 21, wherein the TLR2 agonist is selected from the group consisting of: Pam2Cys, Pam3Cys, Ste2Cys, Lau2Cys, and Oct2Cys.

26. The method or use according to any one of claims 1 to 21, wherein the TLR2 agonist comprises Pam2Cys.

27. The method or use according to any one of claims 1 to 26, wherein the compound comprises a TLR2 agonist and a solubilising agent.

28. The method or use according to claim 27, wherein the TLR2 agonist and solubilising agent are linked.

29. The method or use according to claim 27 or 28, wherein the solubilising agent comprises or consists of a positively or negatively charged group.

30. The method or use according to claim 29, wherein the charged group is a branched or linear peptide.

31. The method or use according to claim 29, wherein the positively charged group comprises at least one positively charged amino acid, preferably an arginine or lysine residue.

32. The method or use according to claim 29, wherein the negatively charged group comprises at least one negatively charged amino acid, preferably a glutamate or aspartate.

33. The method or use according to claim 30, wherein the branched or linear peptide is R4, H4, H8 or E8.

34. The method or use according to claim 30, wherein the branched peptide comprises

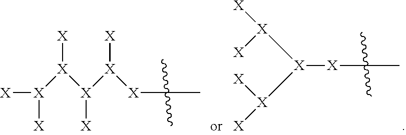

35. The method or use according to claim 27, wherein the solubilising agent comprises polyethyleneglycol (PEG) or R4.

36. The method or use according to claim 27, wherein the solubilising agent comprises polyethyleneglycol (PEG) and R4.

37. The method or use according to claim 35 or 36, wherein the PEG is PEG₁₁ or PEG₁₂.

38. The method or use according to any one of claims 1 to 34, wherein the TLR2 agonist is a compound of formula (I):
A-Y—B (I)
wherein A comprises or consists of a moiety selected from A1 and A2: 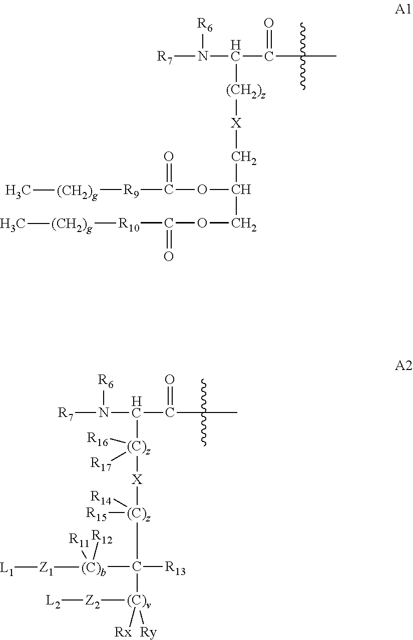
wherein
each z is independently selected from 1 or 2;
each X is independently selected from —S—, —S(═O)— and —S(═O)₂—;
in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and
in moiety A2:
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆,
R₁₇, R₁₈, R₁₉, Rₓ, R_{y}, L₁, L₂, and L₃ is optionally substituted;
Y is 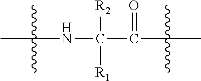
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt or prodrug thereof.

39. The method or use according to any one of claims 1 to 34, wherein the compound comprises or consists of partial structure A1Y′ or A2Y′: 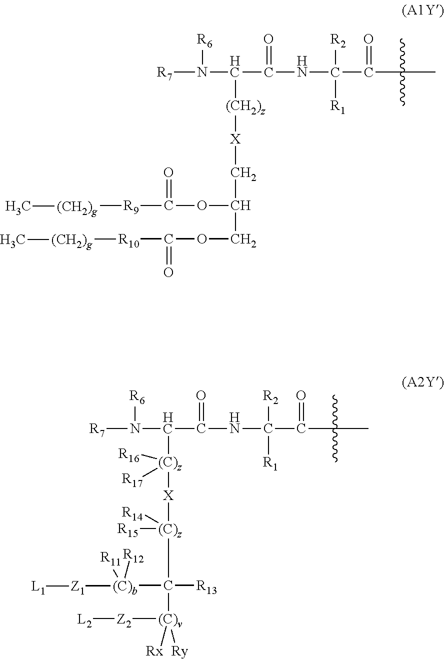
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

40. The method or use according to any one of claims 1 to 34, wherein the TLR2 agonist is a compound comprising a moiety A selected from A1′ and A2 and PEG, wherein the moiety A and PEG are linked by a glycine, serine, homoserine, threonine, phosphoserine, asparagine or glutamine residue, or an ester of a glutamine residue 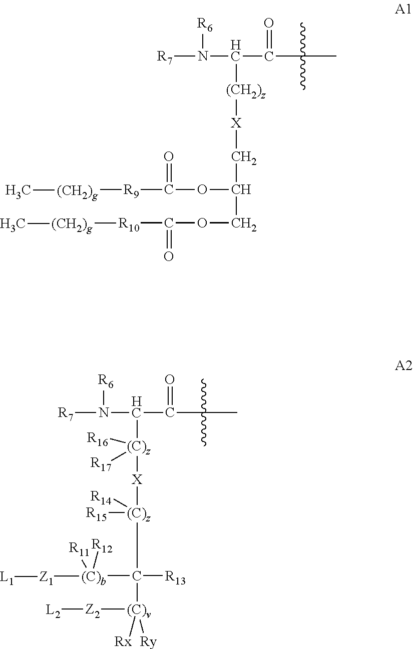
wherein:
each g is independently 10, 11, 12, 13, 14, 15, 16, 17 or 18 each z is independently selected from 1 or 2;
each X is independently selected from —S—, —S(═O)— and —S(═O)₂—;
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted
or a pharmaceutically acceptable salt, solvate or prodrug thereof.

41. A method or use according to any one of claims 1 to 34, wherein the TLR2 agonist is a compound of formula (VIII):
A-Y—NH—(CH₂)ₚ—O—(CH₂—CH₂—O)ₙ—[(CH₂)ₘ—CO-L-]_{q}R₃ (VIII)
wherein
A is a moiety selected from A1 and A2 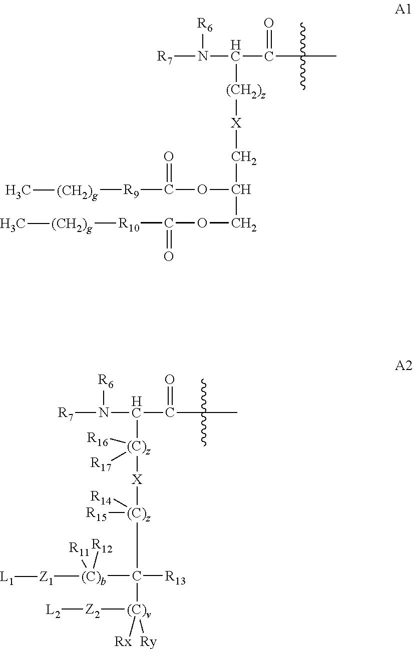
wherein
each z is independently selected from 1 or 2;
each X is independently selected from —S—, —S(═O)— and —S(═O)₂—;
in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and
in moiety A2:
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₁ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is 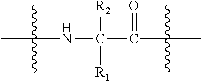
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
n is 3 to 100;
m is 1, 2, 3 or 4;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 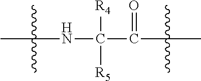
wherein R₄ is H; and
R₅ is the side chain, or second hydrogen of the amino acid,
or a pharmaceutically acceptable salt, solvate or prodrug thereof.

42. A method or use according to any one of claims 1 to 34, wherein the TLR2 agonist is a compound selected from the following Table:

| | Compound |
|---|---|
| Compound Structure | name |
| 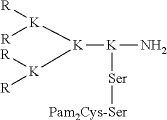 | 001 |
| 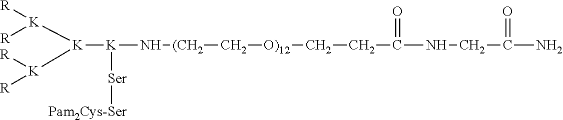 | 002 |
| 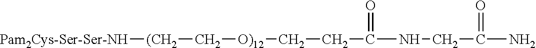 | 003 |
| Pam₂Cys-Ser-Ser-Lys-Lys-Lys-Lys | 004 |
| Pam₂Cys-Ser-Lys-Lys-Lys-Lys | 005 |
| 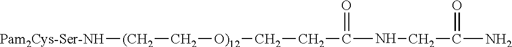 | A101/ compound 1 |
| 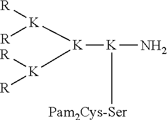 | 007 |
| 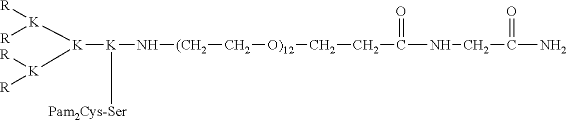 | 008 |
| 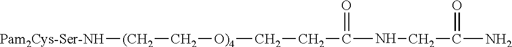 | 009 |
| 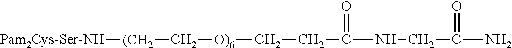 | 010 |
| 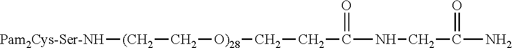 | A105 |
| 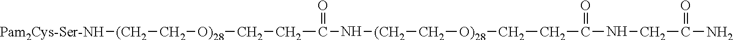 | A106 |
| 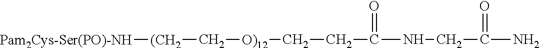 | A104 |
| 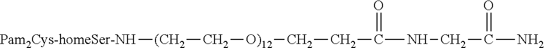 | A103 |
| 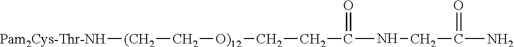 | A102 |
| 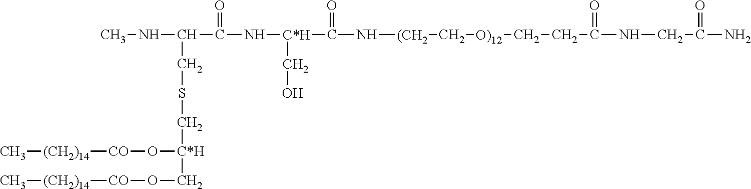 | A109 |
| 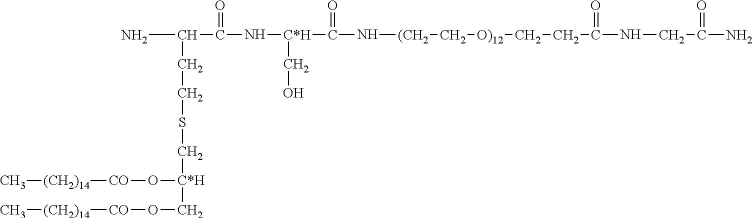 | A110 |
| 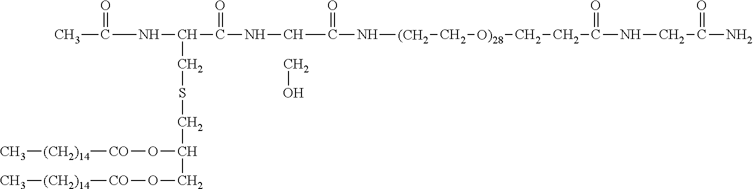 | A111 |
| 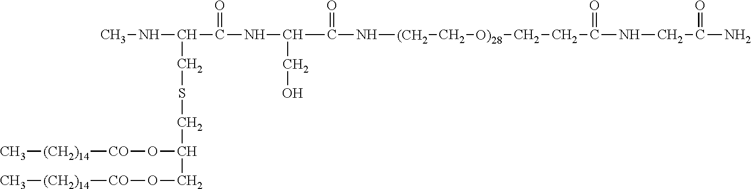 | A112 |
| 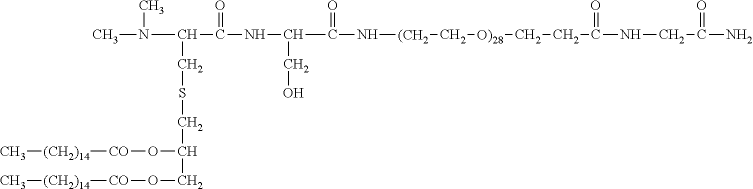 | A113 |
| 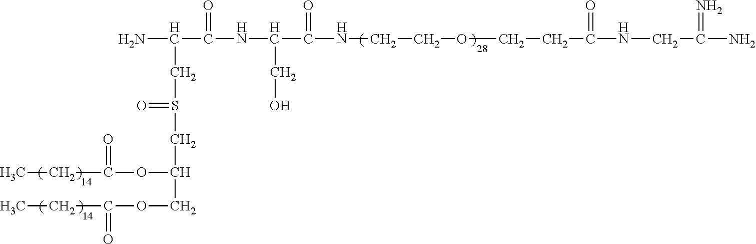 | A114 |
| 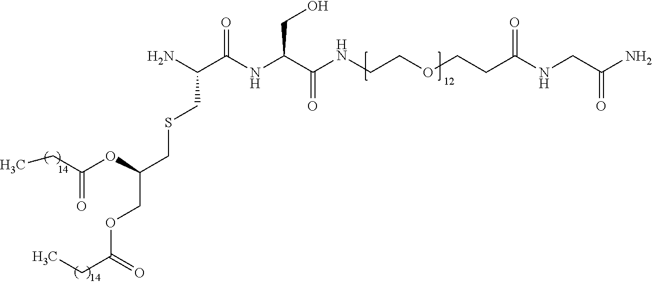 | A107 |
| 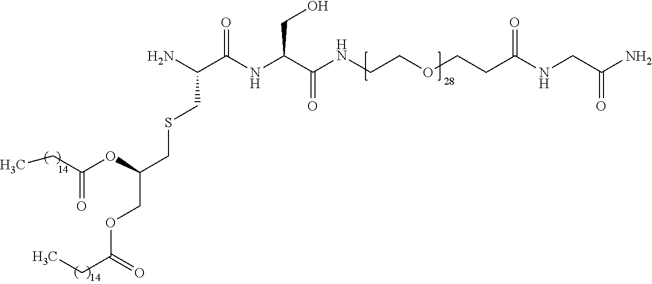 | A108 |
| 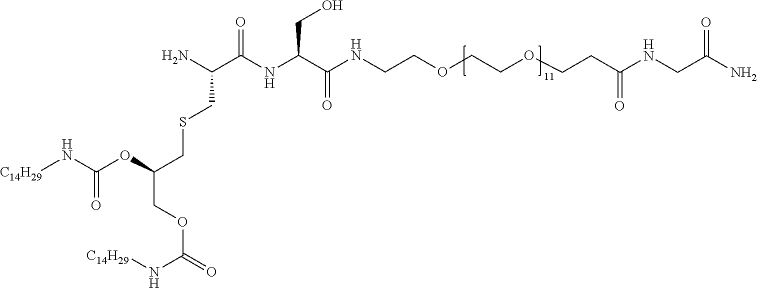 | A115 |
| 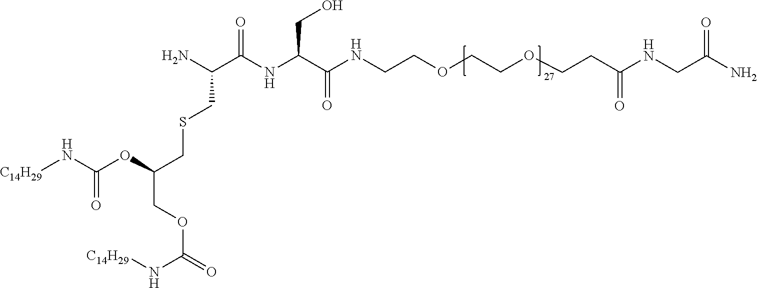 | A116 |
| 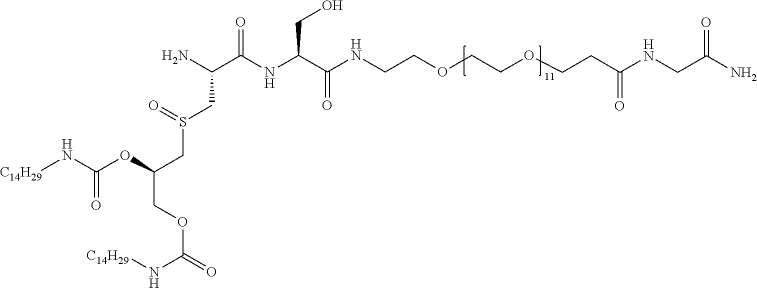 | A117 |
| 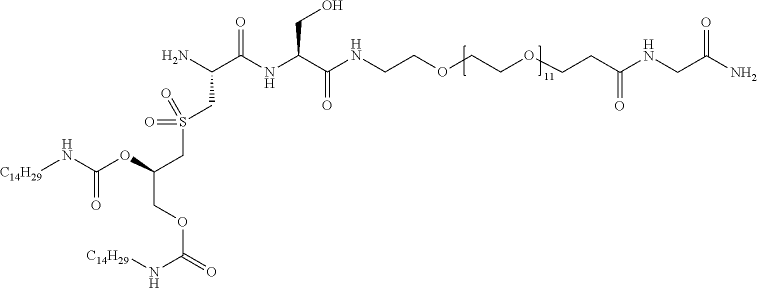 | A118 |
| 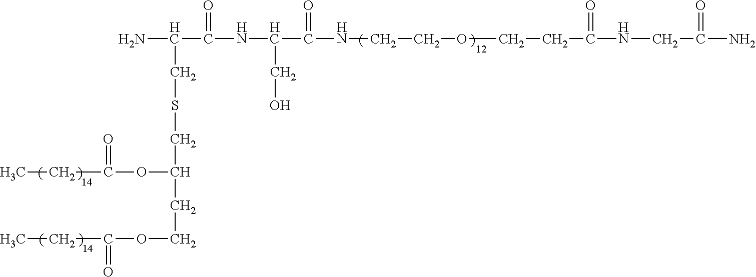 | A201 |
| 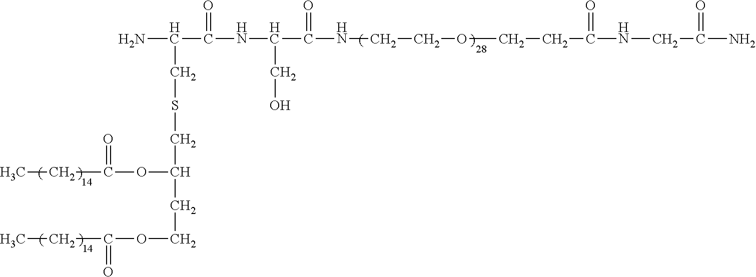 | A202 |
| 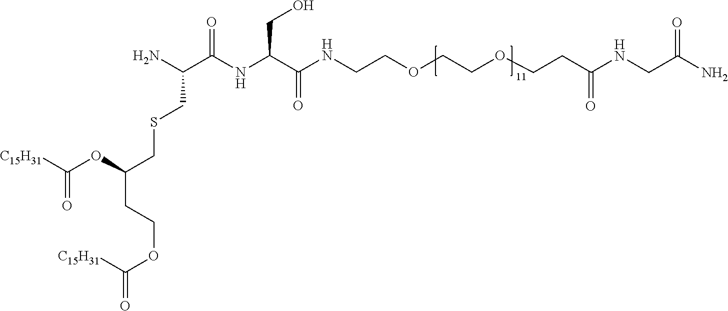 | A203 |
| 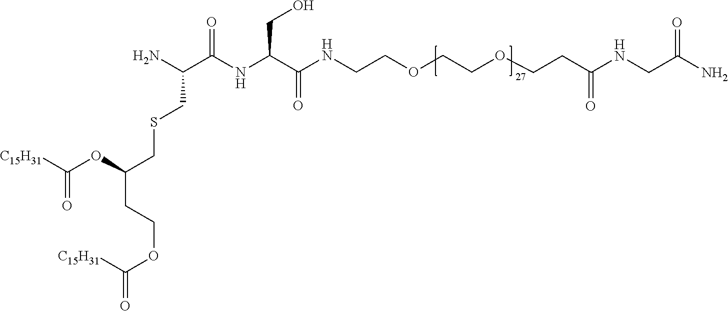 | A204 |
| 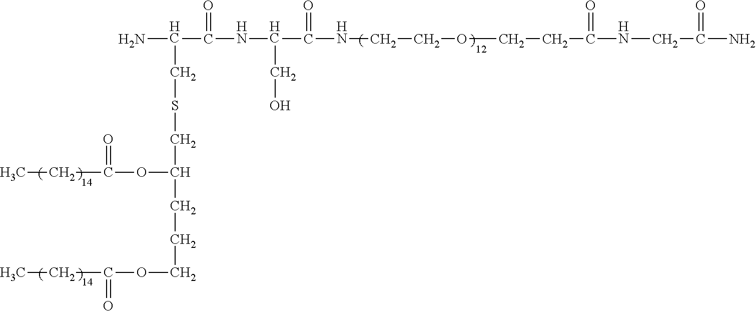 | A205 |
| 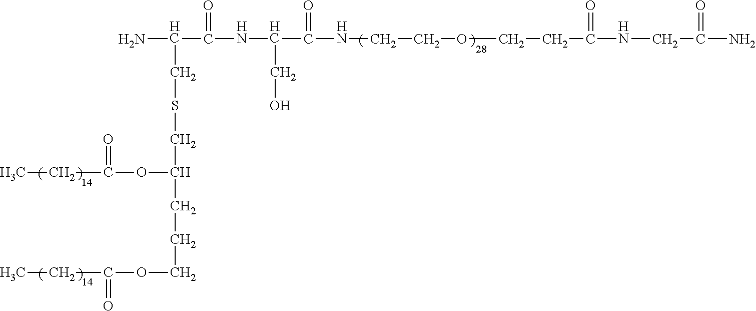 | A206 |
| 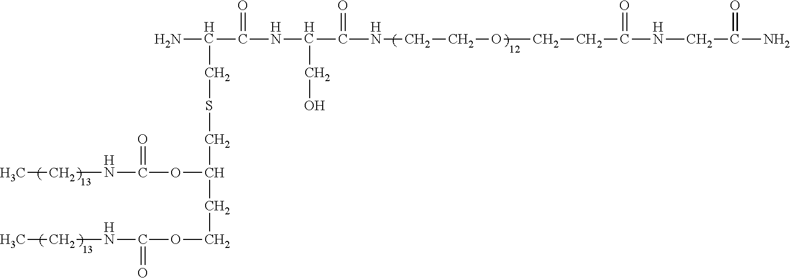 | A207 |
| 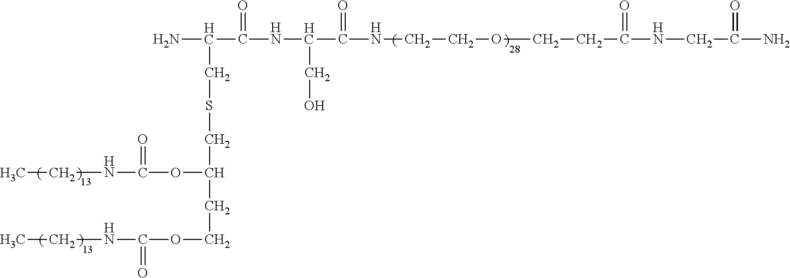 | A208 |
| 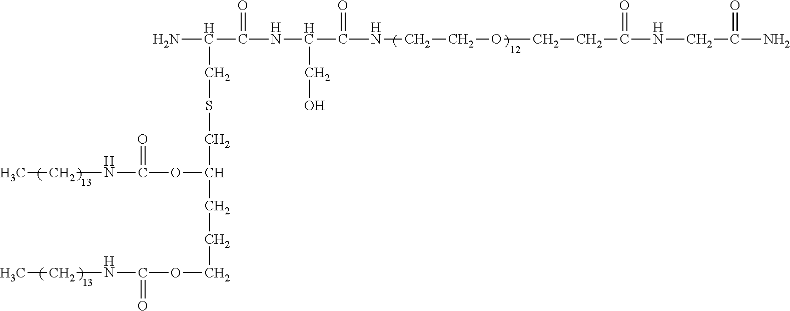 | A209 |
| 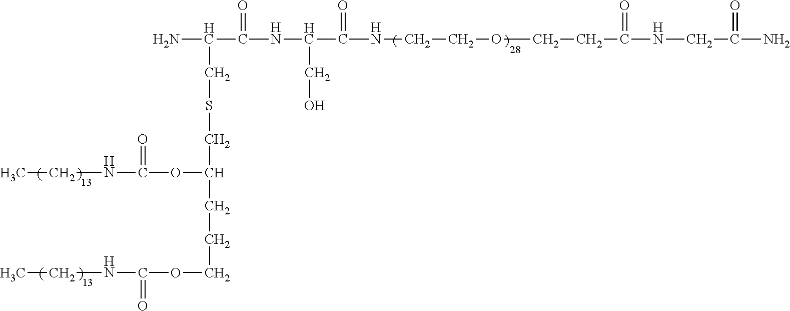 | A210 |
| 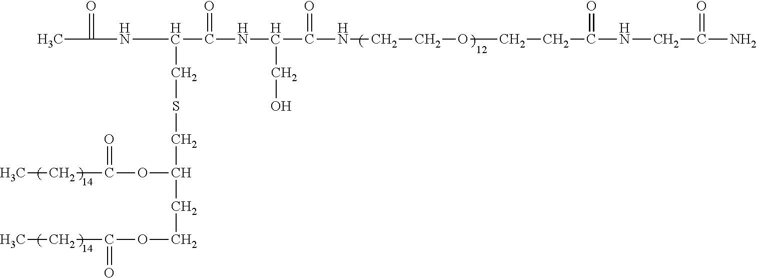 | A211 |
| 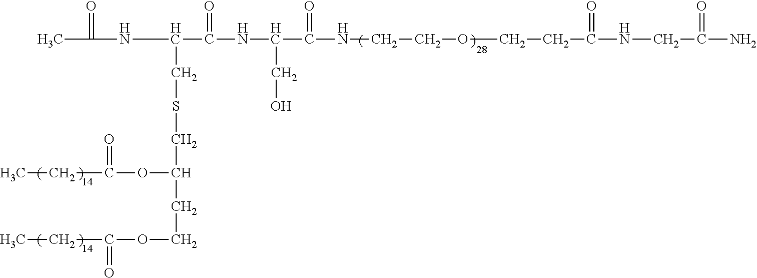 | A212 |
| 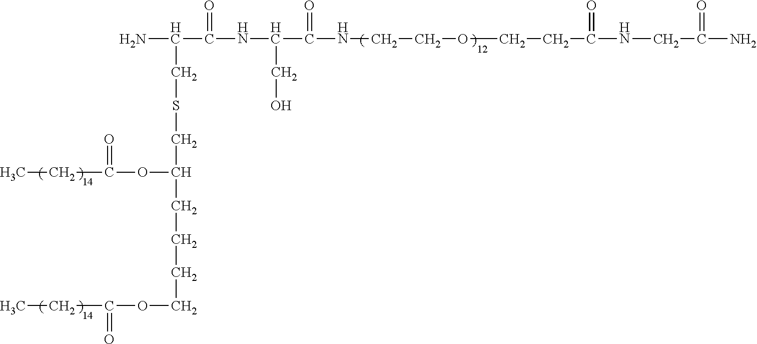 | A213 |
| 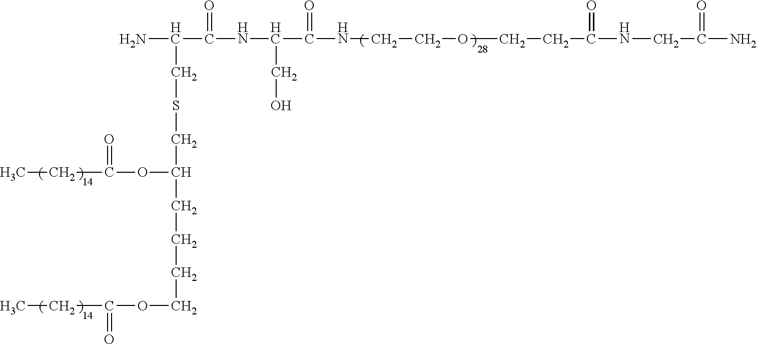 | A214 |
| 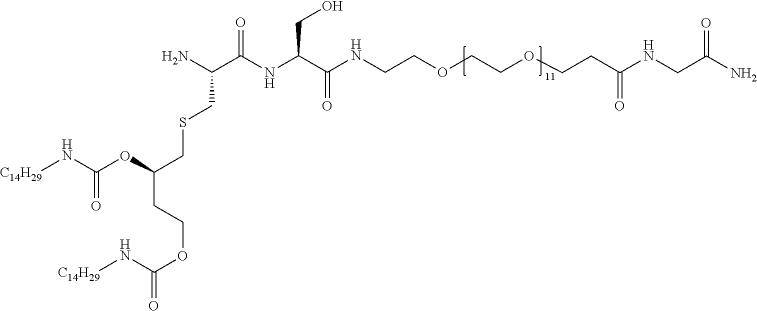 | A215 |
| 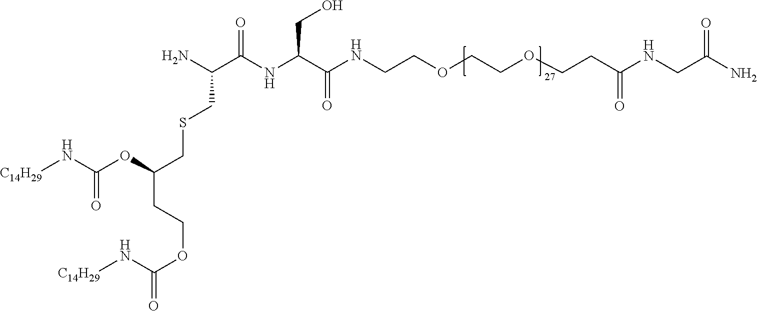 | A216 |
| 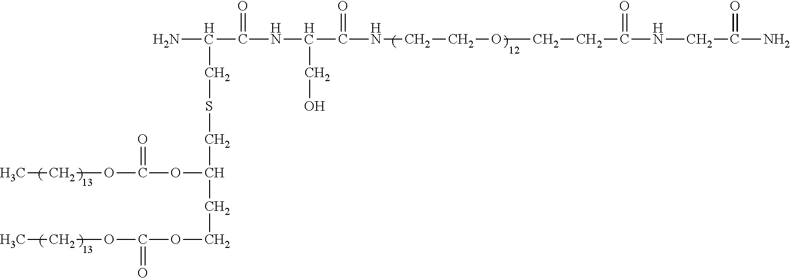 | A217 |
| 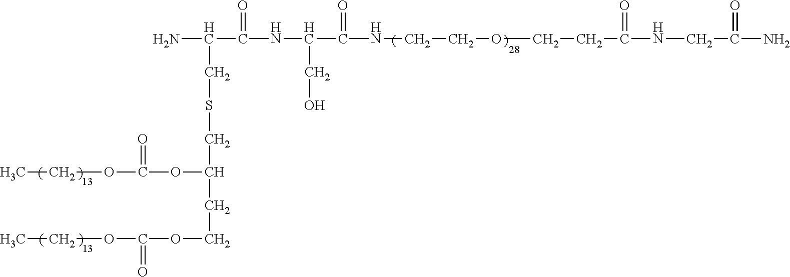 | A218 |
| 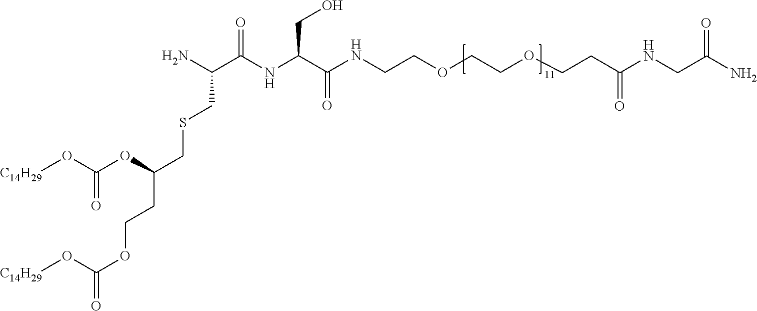 | A219 |
| 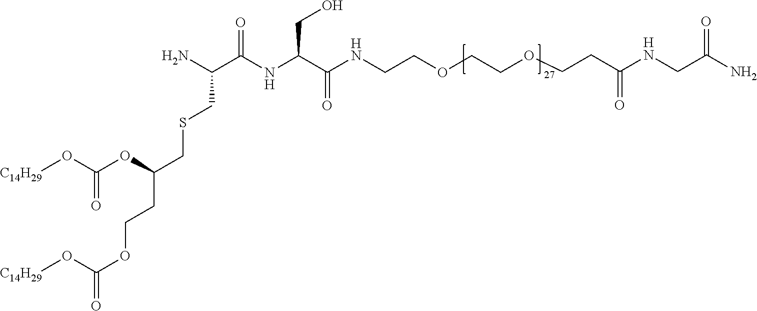 | A220 |
| 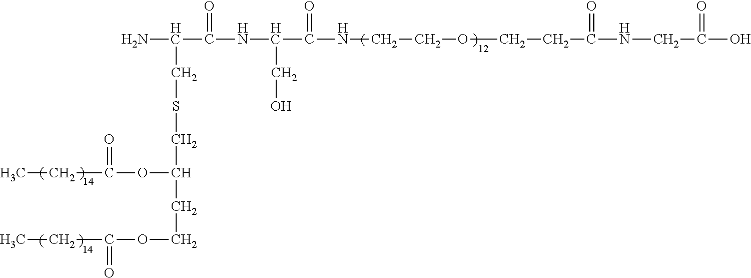 | A221 |
| 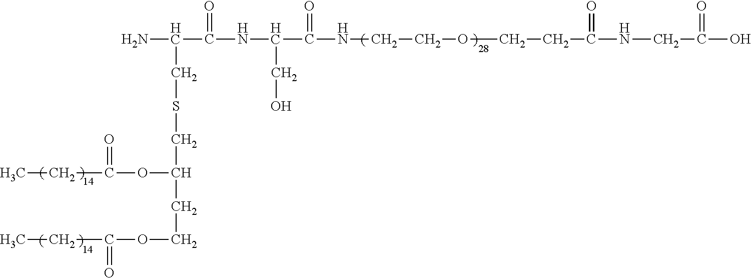 | A222 |
| 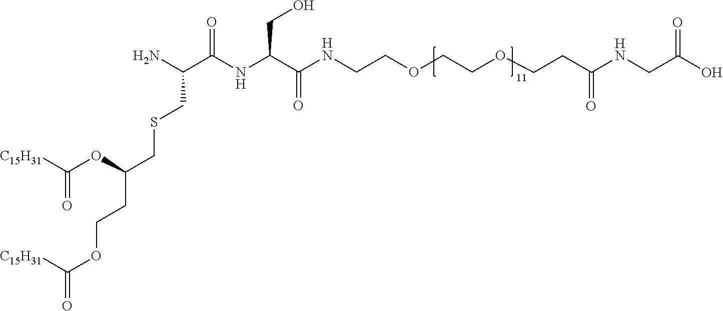 | A223 |
| 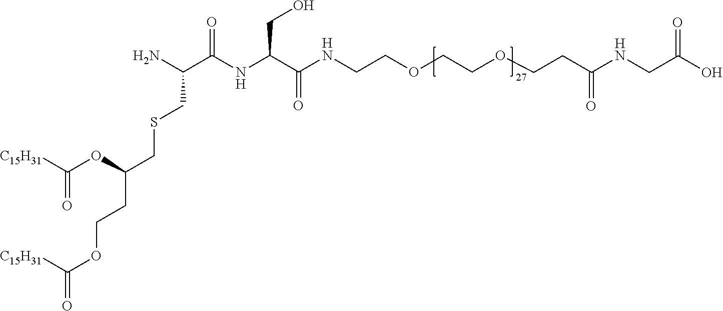 | A224 |
| 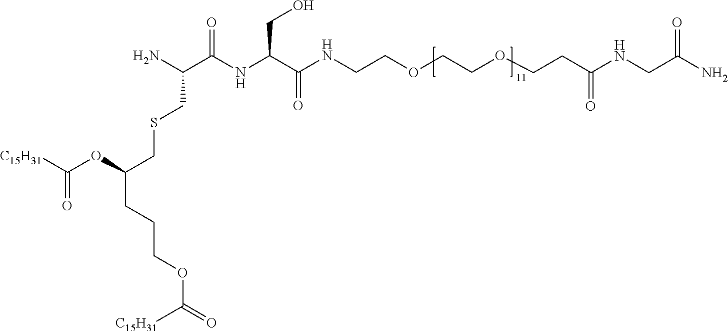 | A225 |
| 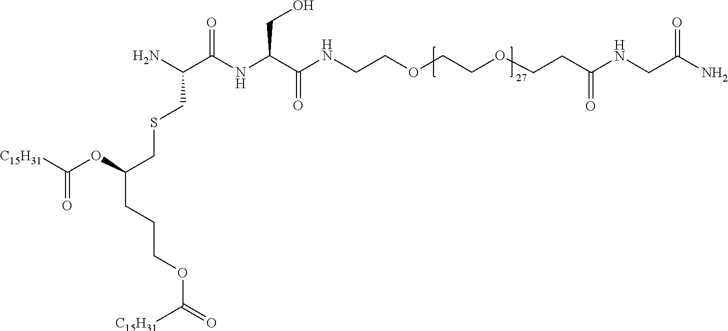 | A226 |
| 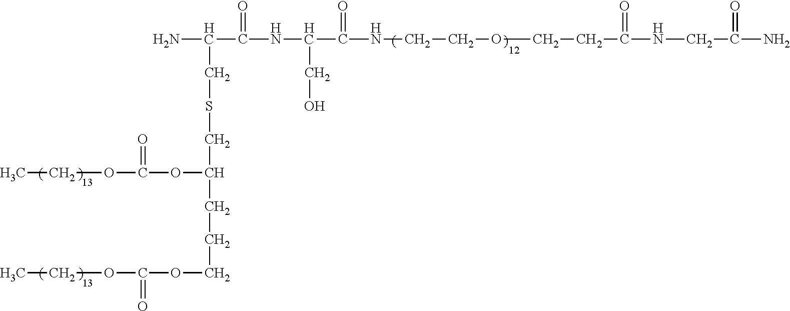 | A227 |
| 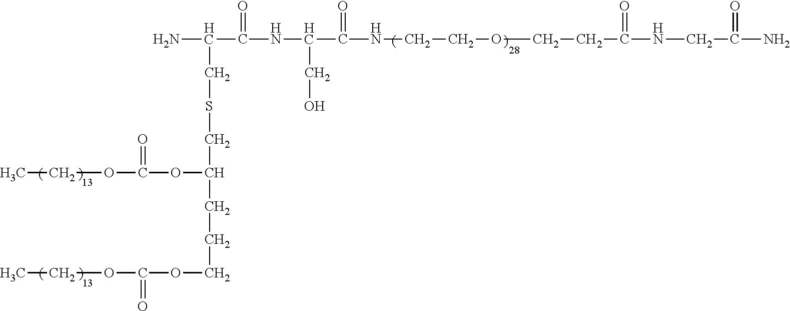 | A228 |
| 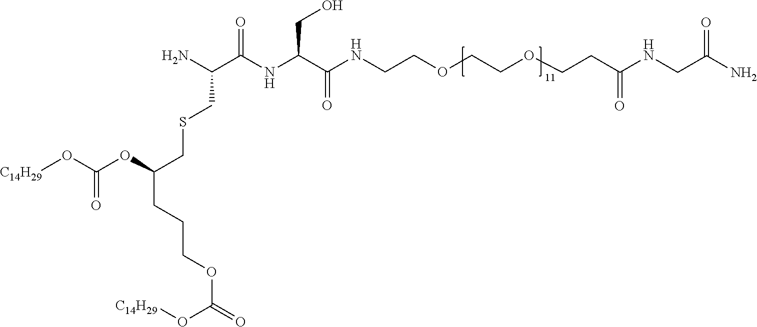 | A229 |
| 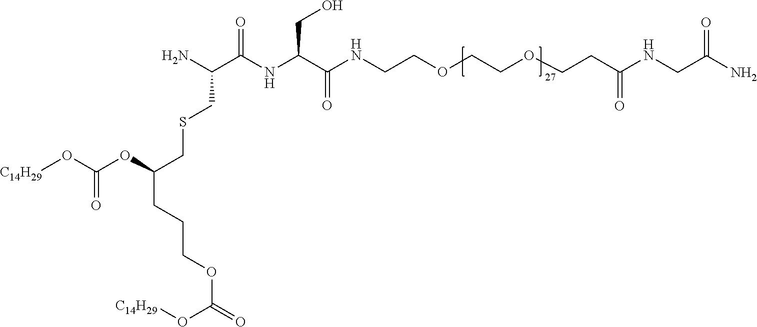 | A230 |
| 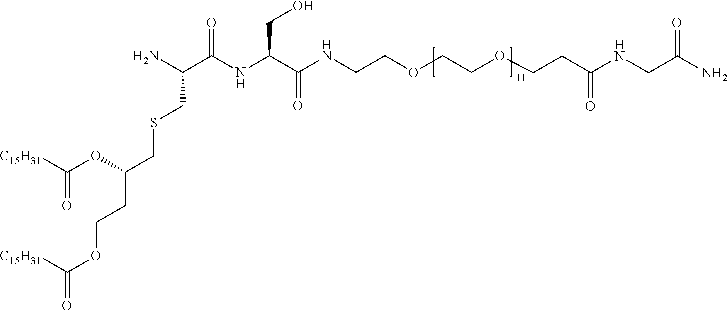 | A231 |
| 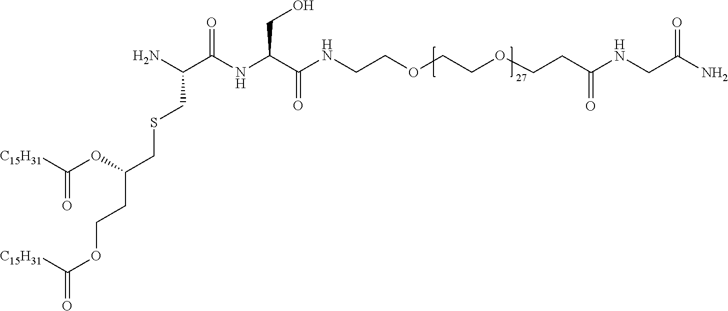 | A232 |

or a pharmaceutically acceptable salt, solvate or prodrug thereof.

44. The method of use of claim 43, wherein the TLR2 agonist is defined by the following formula: 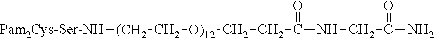
and the immunostimulant is a PD-1, PD-L1 or CTLA-4 antibody.

45. The method of use of claim 43, wherein the TLR2 agonist is defined by the following formula: 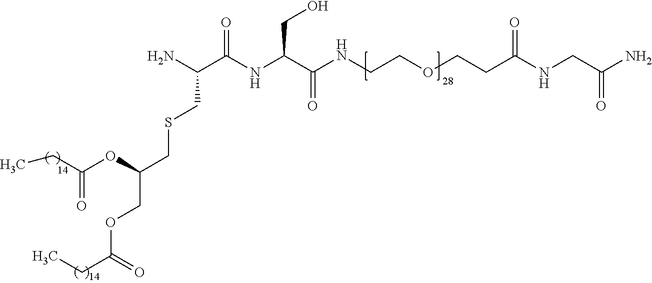
and the immunostimulant is a PD-1, PD-L1 or CTLA-4 antibody.

46. The method or use according to any one of claims 1 to 45, wherein the TLR2 agonist is not Pam3Cys.

47. The method or use according to any one of claims 1 to 46, wherein the therapeutically effective amount of a TLR2 agonist and immunostimulant is administered once daily.

48. The method or use according to any one of claims 1 to 46, wherein the therapeutically effective amount of a TLR2 agonist and immunostimulant is administered once weekly.

49. The method or use according to any one of claims 1 to 48, wherein the therapeutically effective amount of a TLR2 agonist and immunostimulant is suitable for administration intravenously to the subject.

50. The method or use according to any one of claims 1 to 48, wherein the therapeutically effective amount of a TLR2 agonist and immunostimulant is suitable for administration to the respiratory tract, preferably by inhalation.

51. A TLR2 agonist for use in treating, preventing or minimising progression of cancer in a subject in combination with an immunostimulant.

52. An immunostimulant for use in treating, preventing or minimising progression of cancer in a subject in combination with a TLR2 agonist.

53. Use of TLR2 agonist in treating, preventing or minimising progression of cancer in a subject in combination with an immunostimulant.

54. A pharmaceutical composition comprising a therapeutically effective amount of a TLR2 agonist and immunostimulant for use in treating, preventing, or minimising progression of cancer in a subject.

55. The TLR2 agonist of claim 51, the immunostimulant of claim 52, the use of claim 53 or the composition of claim 54, wherein the TLR2 agonist is Pam₂Cys-Ser-PEG defined by the following formula: 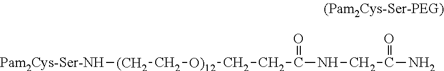
and the immunostimulant is a PD-1, PD-L1 or CTLA-4 antibody.

56. The TLR2 agonist of claim 51, the immunostimulant of claim 52, the use of claim 53 or the composition of claim 54, wherein the TLR2 agonist is defined by the following formula: 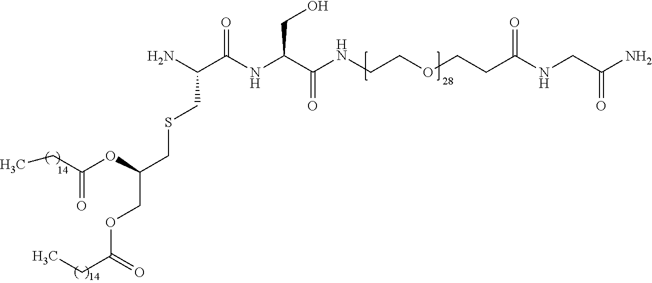
and the immunostimulant is a PD-1, PD-L1 or CTLA-4 antibody.

57. The TLR2 agonist, immunostimulant, use or pharmaceutical composition of any one of claims 51 to 56, wherein the therapeutically effective amount of a TLR2 agonist and immunostimulant is suitable for administration to the respiratory tract of the subject, preferably by inhalation.

58. The TLR2 agonist, immunostimulant, use or pharmaceutical composition of claim 57, wherein the composition is formulated as a nasal spray or as nasal drops.

59. The method, use, TLR2 agonist, immunostimulant or pharmaceutical composition of any one of claims 1 to 58, wherein the TLR2 agonist improves the effectiveness of the immunostimulant in the subject.

60. The method, use, TLR2 agonist, immunostimulant or pharmaceutical composition of claim 59, wherein the TLR2 agonist improves survival, reduces tumour growth and/or reduces metastasis of the tumour in the subject.

61. A kit comprising a TLR2 agonist and/or a checkpoint inhibitor as described in any one of claims 13 to 16 or 19 to 46 for treating, preventing or minimising progression of cancer in a subject.
